SEQUENCE LISTING (92 sequences)

SEQ ID NO: 1 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 10 aa, synthetic construct
EQKLISEEDL

SEQ ID NO: 6 - protein, 111 aa, synthetic construct
IEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 702 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 7 aa, synthetic construct
GFTFSNY

SEQ ID NO: 9 - protein, 8 aa, synthetic construct
RLKSNNYA

SEQ ID NO: 10 - protein, 6 aa, synthetic construct
GNSFAY

SEQ ID NO: 11 - protein, 14 aa, synthetic construct
RSSTGAVTTSNYAN

SEQ ID NO: 12 - protein, 7 aa, synthetic construct
GTNNRAP

SEQ ID NO: 13 - protein, 9 aa, synthetic construct
ALWYSNHWV

SEQ ID NO: 14 - protein, 117 aa, synthetic construct
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSS

SEQ ID NO: 15 - protein, 112 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 16 - protein, 244 aa, synthetic construct
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 17 - DNA, 732 bp, synthetic construct
  source 1..732: mol_type = other DNA
gaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagag

SEQ ID NO: 18 - protein, 55 aa, synthetic construct
VVSHFNDCPLSHDGYCLHDGVCMYIEALDKYACNCVVGYIGERCQYRDLKWWELR

SEQ ID NO: 19 - DNA, 165 bp, synthetic construct
  source 1..165: mol_type = other DNA
gtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgaga

SEQ ID NO: 20 - protein, 42 aa, synthetic construct
RLKIQVRKAAITSYEKSDGVYTGLSTRNQETYETLKHEKPPQ

SEQ ID NO: 21 - protein, 484 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 412 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRG

SEQ ID NO: 23 - protein, 702 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 24 - protein, 192 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCEL

SEQ ID NO: 25 - protein, 104 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAA

SEQ ID NO: 26 - protein, 630 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRG

SEQ ID NO: 27 - protein, 627 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRAPAYQQGQNQLYNELNLGRREEYDVLDKRRG

SEQ ID NO: 28 - protein, 624 aa, Synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKAPAYQQGQNQLYNELNLGRREEYDVLDKRRG

SEQ ID NO: 29 - protein, 621 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSAPAYQQGQNQLYNELNLGRREEYDVLDKRRG

SEQ ID NO: 30 - protein, 618 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAAPAYQQGQNQLYNELNLGRREEYDVLDKRRG

SEQ ID NO: 31 - protein, 4 aa, synthetic construct
RRKR

SEQ ID NO: 32 - protein, 4 aa, synthetic construct
SGSG

SEQ ID NO: 33 - protein, 18 aa, synthetic construct
EGRGSLLTCGDVEENPGP

SEQ ID NO: 34 - protein, 19 aa, synthetic construct
ATNFSLLKQAGDVEENPGP

SEQ ID NO: 35 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 36 - protein, 72 aa, synthetic construct
PTTTPAPRPPTPAPTIASQPLSLRPEACRPAAGGAVHTRGLDFACDIYIWAPLAGTCGVLLLSLVITLYCNH

SEQ ID NO: 37 - protein, 42 aa, synthetic construct
KRGRKKLLYIFKQPFMRPVQTTQEEDGCSCRFPEEEEGGCEL

SEQ ID NO: 38 - protein, 48 aa, synthetic construct
QRRKYRSNKGESPVEPAEPCHYSCPREEEGSTIPIQEDYRKPEPACSP

SEQ ID NO: 39 - protein, 42 aa, synthetic construct
ALYLLRRDQRLPPDAHKPPGGGSFRTPIQEEQADAHSTLAKI

SEQ ID NO: 40 - protein, 38 aa, synthetic construct
CWLTKKKYSSSVHDPNGEYMFMRAVNTAKKSRLTDVTL

SEQ ID NO: 41 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 113 aa, synthetic construct
RVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 43 - protein, 112 aa, synthetic construct
RVKFSRSAEPPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 44 - protein, 272 aa, synthetic construct
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKS

SEQ ID NO: 45 - protein, 917 aa, synthetic construct
MGPGVLLLLLVATAWHGQGGQVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 46 - protein, 407 aa, synthetic construct
MGPGVLLLLLVATAWHGQGGQVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 47 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 48 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 49 - protein, 40 aa, synthetic construct
RVKFSRSADAPAYQQGQNQLYNELNLGRREEYDVLDKRRG

SEQ ID NO: 50 - protein, 37 aa, synthetic construct
RVKFSRAPAYQQGQNQLYNELNLGRREEYDVLDKRRG

SEQ ID NO: 51 - protein, 34 aa, synthetic construct
RVKAPAYQQGQNQLYNELNLGRREEYDVLDKRRG

SEQ ID NO: 52 - protein, 31 aa, synthetic construct
APAYQQGQNQLYNELNLGRREEYDVLDKRRG

SEQ ID NO: 53 - protein, 31 aa, synthetic construct; identical to SEQ ID NO: 52

SEQ ID NO: 54 - protein, 632 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 55 - protein, 414 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 56 - protein, 642 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 57 - protein, 424 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 58 - protein, 52 aa, synthetic construct
YFLGRLVPRGRGAAEAATRKQRITETESPYQELQGQRSDVYSDLNTQRPYYK

SEQ ID NO: 59 - protein, 484 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 60 - protein, 198 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHQRRKYRSNKGESPVEPAEPCHYSCPREEEGSTIPIQEDYRKPEPACSP

SEQ ID NO: 61 - protein, 192 aa, synthetic construct
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHALYLLRRDQRLPPDAHKPPGGGSFRTPIQEEQADAHSTLAKI

SEQ ID NO: 62 - protein, 409 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRAPAYQQGQNQLYNELNLGRREEYDVLDKRRG

SEQ ID NO: 63 - protein, 406 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKAPAYQQGQNQLYNELNLGRREEYDVLDKRRG

SEQ ID NO: 64 - protein, 403 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSAPAYQQGQNQLYNELNLGRREEYDVLDKRRG

SEQ ID NO: 65 - protein, 400 aa, synthetic construct
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAAPAYQQGQNQLYNELNLGRREEYDVLDKRRG

SEQ ID NO: 66 - DNA, 1167 bp, synthetic construct
  source 1..1167: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 67 - protein, 388 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 68 - DNA, 987 bp, synthetic construct
  source 1..987: mol_type = other DNA
atgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgcattgcacatgcaggccctgccccctcgcggctggatccgcggccgcaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggtttataatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 69 - protein, 328 aa, synthetic construct
MRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 70 - skipped

SEQ ID NO: 71 - DNA, 1347 bp, synthetic construct
  source 1..1347: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgcattgcacatgcaggccctgccccctcgcaggcggaaaaggtctgggagtggggctaccaatttctctctcctcaagcaagccggagacgttgaggaaaaccctggacccatgggctggatccgcggccgcaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggtttataatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 72 - protein, 448 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 73 - DNA, 1347 bp, synthetic construct
  source 1..1347: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgatgcattgcacatgcaggccctgccccctcgcaggcggaaaaggtctgggagtggggctaccaatttctctctcctcaagcaagccggagacgttgaggaaaaccctggacccatgggctggatccgcggccgcaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggtttataatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 74 - protein, 448 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPRRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 75 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 76 - protein, 763 aa, synthetic construct
MGWLCSGLLFPVSCLVLLQVASSGNLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGDEPKSCDKTHTCPPCPIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 77 - protein, 253 aa, synthetic construct
MGWLCSGLLFPVSCLVLLQVASSGNLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGDEPKSCDKTHTCPPCPIYIWAPLAGTCGVLLLSLVITLYCNHKRGRKKLLYIFKQPFMRPVQTTQEEDGCSCRFPEEEEGGCEL

SEQ ID NO: 78 - protein, 797 aa, synthetic construct
MGWLCSGLLFPVSCLVLLQVASSGNLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGDGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 79 - protein, 287 aa, synthetic construct
MGWLCSGLLFPVSCLVLLQVASSGNLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGDGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCEL

SEQ ID NO: 80 - protein, 782 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 81 - protein, 272 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCEL

SEQ ID NO: 82 - protein, 819 aa, synthetic construct
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIAGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 83 - protein, 309 aa, synthetic construct
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIAGGGGSGTPSSDAVSRLEEEMRKLQATVQELQKRLDRLEETVQAKGGGGIISFFLALTSTALLFLLFFLTLRFSVVKRGRKKLLYIFKQPFMRPVQTTQEEDGCSCRFPEEEEGGCEL

SEQ ID NO: 84 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 85 - protein, 52 aa, Homo sapiens; identical to SEQ ID NO: 58

SEQ ID NO: 86 - protein, 155 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAH

SEQ ID NO: 87 - protein, 192 aa, Homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIA

SEQ ID NO: 88 - protein, 205 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 89 - DNA, 2292 bp, synthetic construct
  source 1..2292: mol_type = other DNA
atgggatggctgtgcagcggactgctgttccccgtgtcctgtctggtgctgctgcaggtggccagctccggaaacctgtttaatcaggaggtgcagatccctctgaccgagtcctactgcggcccatgtcccaagaactggatctgctacaagaacaactgttaccagttctttgatgagtctaagaactggtatgagagccaggcctcctgcatgtctcagaatgcctccctgctgaaggtgtactctaaggaggaccaggatctgctgaagctggtgaagagctatcactggatgggcctggtgcacatccctacaaacggctcctggcagtgggaggacggcagcatcctgtccccaaatctgctgaccatcatcgagatgcagaagggcgattgcgccctgtacgcctctagctttaagggctatatcgagaactgctccacccctaatacatacatctgtatgcagaggaccgtgggaggaggaggaagcggaggaggaggcgacgagccaaagtcctgtgataagacccacacatgcccaccctgtcctatctacatctgggcaccactggcaggaacatgcggcgtgctgctgctgagcctggtcatcaccctgtattgtaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctaa

SEQ ID NO: 90 - DNA, 2394 bp, synthetic construct
  source 1..2394: mol_type = other DNA
atgggatggctgtgcagcggactgctgttccccgtgtcctgtctggtgctgctgcaggtggccagctccggaaacctgtttaatcaggaggtgcagatccctctgaccgagtcctactgcggcccatgtcccaagaactggatctgctacaagaacaactgttaccagttctttgatgagtctaagaactggtatgagagccaggcctcctgcatgtctcagaatgcctccctgctgaaggtgtactctaaggaggaccaggatctgctgaagctggtgaagagctatcactggatgggcctggtgcacatccctacaaacggctcctggcagtgggaggacggcagcatcctgtccccaaatctgctgaccatcatcgagatgcagaagggcgattgcgccctgtacgcctctagctttaagggctatatcgagaactgctccacccctaatacatacatctgtatgcagaggaccgtgggaggaggaggaagcggaggaggaggcgacggcggagggggcagcggcacccctagcagcgacgccgtgagccggctggaggaggagatgcggaagctgcaggccaccgtgcaggagctgcagaagcggctggaccggctggaggagaccgtgcaggccaagggcggaggaggcatcatcagcttcttcctggccctgaccagcaccgccctgctgttcctgctgttcttcctgaccctgcggttcagcgtggtgaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctaa

SEQ ID NO: 91 - DNA, 2349 bp, synthetic construct
  source 1..2349: mol_type = other DNA
atgcagatcccccaggctccctggcccgtggtgtgggccgtgctgcagctgggctggcggcctggctggttcctggacagccccgaccggccctggaaccctcccaccttcagccccgccctgctggtggtgaccgagggcgacaacgccaccttcacctgcagcttcagcaacaccagcgagagcttcgtgctgaactggtaccggatgagccccagcaaccagaccgacaagctggctgccttccccgaggaccggagccagcctggccaggactgccggttccgggtgacccagctgcccaacggacgggacttccacatgagcgtggtgcgggctcggaggaacgacagcggcacctacctgtgcggagccatcagcctggctcccaaggcccagatcaaggagagcctgcgggctgagctgcgggtgaccgagcggagggccgaggtgcccaccgcccacggcggagggggcagcggcacccctagcagcgacgccgtgagccggctggaggaggagatgcggaagctgcaggccaccgtgcaggagctgcagaagcggctggaccggctggaggagaccgtgcaggccaagggcggaggaggcatcatcagcttcttcctggccctgaccagcaccgccctgctgttcctgctgttcttcctgaccctgcggttcagcgtggtgaagaggggcaggaagaagctgctgtacatcttcaagcagcccttcatgcggcctgtgcagaccacccaggaggaggacggctgcagctgccggttccccgaggaggaggagggcggatgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctaa

SEQ ID NO: 92 - DNA, 2460 bp, synthetic construct
  source 1..2460: mol_type = other DNA
atggcctggagggccctgcaccctctgctgctgctgctgctgctgttccccggcagccaggcccagagcaaggcccaggtgctgcagagcgtggctggccagaccctgaccgtgcggtgccagtaccctcccaccggcagcctgtacgagaagaagggctggtgcaaggaggccagcgccctggtgtgcatccggctggtgaccagcagcaagcctcggacaatggcctggaccagccggttcaccatctgggacgaccccgacgctggcttcttcaccgtgaccatgaccgacctgcgggaggaggacagcggccactactggtgccggatctacaggcccagcgacaacagcgtgagcaagagcgtgcggttctacctggtggtgagccctgccagcgccagcacccagaccagctggacccctcgggacctggtgagcagccagacccagacccagagctgcgtgcctcccaccgccggagcccggcaggctcccgagagccccagcaccatccccgtgcccagccagccccagaacagcaccctgcggcctggacccgctgcccctatcgccggcggagggggcagcggcacccctagcagcgacgccgtgagccggctggaggaggagatgcggaagctgcaggccaccgtgcaggagctgcagaagcggctggaccggctggaggagaccgtgcaggccaagggcggaggaggcatcatcagcttcttcctggccctgaccagcaccgccctgctgttcctgctgttcttcctgaccctgcggttcagcgtggtgaagaggggcaggaagaagctgctgtacatcttcaagcagcccttcatgcggcctgtgcagaccacccaggaggaggacggctgcagctgccggttccccgaggaggaggagggcggatgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctaa

(19) United States
(12) Patent Application Publication
(10) Pub. No.: US 2026/0201060 A1
(43) Pub. Date: Jul. 16, 2026

(54) PARALLEL CHIMERIC ANTIGEN RECEPTORS (pCARs) AND ADAPTOR CHIMERIC ANTIGEN RECEPTORS COMPRISING ALTERNATIVE SIGNALLING DOMAINS AND METHODS OF USE THEREOF

(71) Applicants: KING'S COLLEGE LONDON, London (GB); Leucid Bio Ltd, Great Cambourne, Cambridge (GB)

(72) Inventors: Phoebe Dunbar, Great Cambourne, Cambridge (GB); Marc Davies, Great Cambourne, Cambridge (GB); Maya Glover, Great Cambourne, Cambridge (GB); John Maher, Strand, London (GB)

(21) Appl. No.: 19/471,651

(22) PCT Filed: Apr. 5, 2024
(86) PCT No.: PCT/EP2024/059381

§ 371 Date: Oct. 1, 2025

(30) Foreign Application Priority Data
Apr. 5, 2023 (GB) 2305067.7
Mar. 14, 2024 (GB) 2403716.0

(51) Int. Cl. C07K 16/30; A61K 40/11; A61K 40/31; A61K 40/42; A61P 35/00; C07K 14/725; C07K 16/28

(52) U.S. Cl. CPC C07K 16/3092; A61K 40/11; A61K 40/31; A61K 40/4202; A61K 40/4257; A61P 35/00; C07K 14/7051; C07K 16/2851; C07K 2319/02

(57) ABSTRACT

Provided herein are second generation chimeric antigen receptors (CARs), parallel CARs (pCARs), and adaptor CARs comprising an intracellular signalling domain modified for optimal signalling. Also provided herein are compositions and methods for improving anti-tumour efficacy and restimulation capacity of CAR T-cells, pCAR T-cells, and adaptor CAR T-cells.

### 1. SEQUENCE LISTING

The instant application contains a Sequence Listing which has been submitted electronically and is hereby incorporated by reference in its entirety. Said XML copy, created on 4 Apr. 2024, is named P4861PC00 Sequence Listing, and is 115,824 bytes in size.

### 2. BACKGROUND

Chimeric antigen receptors (CARs), which are at times referred to as artificial T cell receptors, chimeric T cell receptors (cTCR), or chimeric immunoreceptors, are engineered receptors now well known in the art. They are used primarily to transform immune effector cells, in particular T cells, to provide those cells with a desired engineered specificity. Adoptive cell therapies using CAR-T cells are particularly under investigation in the field of cancer therapy. In these therapies, T cells are removed from a patient and modified so that they express CARs specific to the antigens found in a particular form of cancer. The CAR-T cells, which can then recognize and kill the cancer cells, are reintroduced into the patient.

First generation CARs provide a TCR-like signal, most commonly using a CD3 zeta (CD3ζ) intracellular signalling domain, and thereby elicit tumouricidal functions. However, the engagement of CD3ζ-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB.

Second generation CARs have been constructed to transduce a functional antigen-dependent co-stimulatory signal in human primary T cells in addition to antigen-dependent TCR-like signal, permitting T cell proliferation in addition to tumouricidal activity. Second generation CARs most commonly provide co-stimulation using co-stimulatory domains (synonymously, co-stimulatory signalling regions) derived from CD28 or 4-1BB. The combined delivery of co-stimulation plus a CD3ζ signal renders second-generation CARs clearly superior in terms of function as compared to their first generation counterparts (CD3ζ signal alone). An example of a second generation CAR is found in U.S. Pat. No. 7,446,190, incorporated herein by reference.

More recently, so-called third generation CARs have been prepared. These combine multiple co-stimulatory domains (synonymously, co-stimulatory signalling regions) with a TCR-like signalling domain in cis, such as CD28+4-1BB+CD3ζ or CD28+OX40+CD3ζ to further augment potency. In the third generation CARs, the co-stimulatory domains are aligned in series in the CAR endodomain and are generally placed upstream of CD3ζ or its equivalent.

In general, however, the results achieved with these third generation CARs have been disappointing, showing only a marginal improvement over second generation configurations, with some third generation CARs being inferior to second generation configurations.

We have recently described a new format in which immuno-responsive cells such as T cells are engineered to express two constructs in parallel, a 2^{nd} generation CAR and a chimeric co-stimulatory receptor (CCR). The 2^{nd} generation CAR comprises, from C-terminus to N-terminus (from intracellular to extracellular), the following domains: (a) a signalling region; (b) a co-stimulatory signalling region; (c) a transmembrane domain; and (d) a first binding element that specifically interacts with a first epitope on a first target antigen. The CCR comprises, from C-terminus to N-terminus (from intracellular to extracellular), (a) a co-stimulatory signalling region which is different from the co-stimulatory signalling region of the CAR; (b) a transmembrane domain; and (c) a second binding element that specifically interacts with an epitope on a target antigen. The CAR and CCR may recognize an identical epitope, different epitopes on the same antigen, or epitopes found on two distinct antigens. Unlike the CAR, the CCR lacks a TCR-like signalling region such as CD3ζ. These parallel CAR (pCAR)-engineered T cells demonstrate superior activity and resistance to exhaustion as compared to first generation CAR-T cells, second generation CAR-T cells, and third generation CAR-T cells. See US pre-grant publication 2019/0002521 and PCT publications WO 2020/183158 and WO 2021/0383036, each of which is incorporated herein by reference in its entirety.

These properties of pCAR-T cells make them attractive candidates for treatment of refractory malignancies, where first, second, and third generation CAR-T cells show limited efficacy in part due to T cell exhaustion. However, there is need for further development of this technology to improve its therapeutic efficacy.

### 3. SUMMARY

In one aspect, described herein is a parallel chimeric antigen receptor (pCAR) comprising:
(1) a second-generation chimeric antigen receptor (second-generation CAR) comprising:
a) an intracellular signalling domain comprising a modified CD3ζ polypeptide;
b) a co-stimulatory signalling region;
c) a transmembrane domain; and
d) a first binding element that specifically interacts with a first epitope on a first target antigen,
wherein the modified CD3ζ polypeptide comprises an unmodified immunoreceptor tyrosine-based activation motif 1 (ITAM1), and one or more mutations in ITAM2 and/or ITAM3; and
(2) a chimeric co-stimulatory receptor (CCR) comprising at least one CCR polypeptide comprising:
e) a co-stimulatory signalling region which is different from that of (b);
f) a transmembrane domain; and
g) a second binding element that specifically interacts with a second epitope on a second target antigen. The disclosure also provides:
   one or more polynucleotide constructs encoding the pCAR of the invention,
   one or more expression vectors comprising the one or more polynucleotide constructs of the invention,
   an immunoresponsive cell comprising the pCAR of the invention, the one or more polynucleotide constructs of the invention, or the one or more expression vectors of the invention,
   a pharmaceutical composition comprising the one or more polynucleotide constructs of the invention, the one or more expression vectors of the invention, or a population of immunoresponsive cells of the invention and an excipient,
   the pCAR of the invention, the one or more polynucleotide constructs of the invention, the one or more expression vectors of the invention, the immunoresponsive cell of the invention, or the pharmaceutical composition of the invention for use in therapy, and
   the pCAR of the invention, the one or more polynucleotide constructs of the invention, the one or more expression vectors of the invention, the immunoresponsive cell of the invention, or the pharmaceutical composition of the invention for use in the treatment of cancer.

In another aspect, described herein is a fusion polypeptide comprising a DNAX-activating protein 10 (Dap10) polypeptide or a functional variant thereof and a modified CD3ζ polypeptide that comprises an unmodified immunoreceptor tyrosine-based activation motif 1 (ITAM1), and one or more mutations in ITAM2 and/or ITAM3.

In another aspect, described herein is a fusion polypeptide comprising (i) a DNAX-activating protein 10 (Dap10) polypeptide, or a functional variant thereof, and (ii) a modified CD3ζ polypeptide that comprises an unmodified immunoreceptor tyrosine-based activation motif 1 (ITAM1), and one or more mutations in ITAM2 and/or ITAM3.

### 4. BRIEF DESCRIPTION OF THE DRAWINGS

These and other features, aspects, and advantages of the present invention will become better understood with regard to the following description, and accompanying drawings, where:

FIGS. 1A, 1B and 1C illustrate schematic diagrams of CAR and pCAR constructs. FIG. 1A shows the schematic diagrams of TBB/H, H2, TBB/H 1XX, and H2 1XX constructs. H2 comprises from intracellular to extracellular, a CD3ζ signalling region, CD28 co-stimulatory and transmembrane domains, and a human MUC1-targeting HMFG2 single chain antibody (scFv) domain. TBB/H is a pCAR that utilizes the MUC1-targeting second-generation “H2” CAR. The chimeric co-stimulatory receptor (CCR) in the TBB/H pCAR has a pan ErbB-specific T1E binding domain fused to CD8α transmembrane domain and a 4-1BB co-stimulatory domain. H2 1XX is a modified version of the H2 CAR that comprises mutations (Y to F) in the two distal CD3ζ immunoreceptor tyrosine-based activation motifs (ITAMs) to create a single functional ITAM (proximal). TBB/H 1XX is a modified version of the TBB/H pCAR that comprises mutations (Y to F) in the two distal CD3ζ immunoreceptor tyrosine-based activation motifs (ITAMs) to create a single functional ITAM (proximal).

FIG. 1B shows the schematic diagrams of the TBB/H-1, TBB/H-1Δ3, TBB/H-1Δ6, TBB/H-1Δ9, and TBB/H-1Δ9 YRS pCAR constructs. TBB/H-1 is a modified version of TBB/H in which the CD3ζ polypeptide is truncated to remove the two distal ITAMS (ITAM2 and ITAM3). TBB/H-1Δ3, TBB/H-1Δ6 and TBB/H-1Δ9 are modified versions of TBB/H-1 that further have 3, 6, or 9 amino acid residues deleted upstream of the residual ITAM within the truncated CD3ζ polypeptide, respectively. TBB/H-1Δ9 YRS is a modified version of TBB/H-1Δ9 that further comprises deletion of the C-terminal YRS motif of the CD28 co-stimulatory region.

FIG. 1C shows schematic diagrams of the FcεR1γ and DAP12 pCAR constructs, which are modified versions of the TBB/H pCAR in which the CD3ζ intracellular signalling domain of the TBB/H pCAR is replaced with the activation domain from FcεR1γ or DAP12, each of which contains a single ITAM.

FIGS. 2A-2E show results of antigen restimulation assays performed on BXPC3 tumour cell monolayers with T cells expressing the CARs and pCARs schematized in FIG. 1A. FIGS. 2A-2B show the H2 1XX CAR-T cells (open diamond) had anti-tumour activity for more restimulation cycles compared to H2 CAR-T cells, with equivalence to TBB/H pCAR-T cells. FIGS. 2C-2E show that TBB/H 1XX-T cells had better restimulation capability on BXPC3 tumour monolayers with increased proliferative capacity compared to TBB/H-T cells.

FIGS. 3A-3B show results of antigen restimulation assays performed on BXPC3 tumour cell monolayers with T cells expressing the pCARs schematized in FIGS. 1A and 1B. FIG. 3A shows that TBBH/H 1XX-pCAR T cells have better anti-tumour efficacy compared to pCAR-T cells expressing a truncated CD3ζ signalling domain. FIG. 3B shows that pCAR-T cells expressing truncated CD3ζ had reduced proliferative capacity compared to TBB/H 1XX pCAR-T cells.

FIGS. 4A-4C show results of antigen restimulation assays performed on BXPC3 tumour cell monolayers with T cells expressing the CAR and pCARs schematized in FIGS. 1A and 1C. TBB/H 1XX pCAR-T cells (large inverted triangle) had superior anti-tumour activity (FIG. 4A), proliferation potential (FIG. 4B), and restimulation capacity (FIG. 4C) compared to pCAR-T cells expressing the DAP12 (square) or FcεR1γ (open diamond) signalling domain.

FIGS. 5A-5D are schematic illustrations of NKG2D_bb/H 1XX, NKG2D(trimer)/H 1XX, Pbb(trimer)/H 1XX, and 44bb(trimer)/H 1XX pCAR constructs. The second-generation CAR in each pCAR construct (“H 1XX”) comprises from intracellular to extracellular, a CD3ζ signalling region that comprises mutations (Y to F) in the two distal CD3ζ ITAMs to create a single functional ITAM (proximal), CD28 co-stimulatory and transmembrane domains, and a human MUC1-targeting HMFG2 single chain antibody (scFv) domain. FIG. 5A is a schematic of the NKG2D_bb/H 1XX pCAR construct. FIG. 5B is a schematic of the NKG2Dbb(trimer)/H 1XX pCAR construct. FIG. 5C is a schematic of the Pbb(trimer)/H 1XX pCAR construct. FIG. 5D is a schematic of the 44bb(trimer)/H 1XX pCAR construct.

FIGS. 6A-6I show the growth of firefly luciferase (ffLUC)-expressing BXPC3 tumour xenografts in mice treated with CAR or pCAR-T cells, as ascertained by bioluminescence imaging (BLI). Results shown are from mice treated with NKG2D_bb/H 1XX pCAR-T cells (FIG. 6A), NKG2D(trimer)/H 1XX pCAR-T cells (FIG. 6B), Pbb(trimer)/H 1XX pCAR-T cells (FIG. 6C), 44bb(trimer)H 1XX pCAR-T cells (FIG. 6D), TBB/H pCAR-T cells (FIG. 6E), TBB/H 1XX pCAR-T cells (FIG. 6F), H2 1XX CAR-T cells (FIG. 6G). Mice treated with CD19-specific F28z transduced T-cells (FIG. 6H) or PBS (FIG. 6I) served as negative controls. Insets in FIGS. 6A-6G are schematics of the CAR or pCAR administered.

FIG. 7 is a survival curve of mice assessed for tumour growth in FIGS. 6A-6I.

FIG. 8 shows schematic illustrations of adaptor CAR constructs NKG2D/Dap10-12, NKG2D/Dap10-CD3ζ and NKG2D/Dap10-CD3ζ (1XX) and the linear CAR NKG2D-CD3ζ.

FIGS. 9A-9C G show results of assays assessing NKG2D ligand targeting adaptor CARs and linear CAR for transduction efficiency on day 3 and day 10 post gene transfer (FIG. 9A), CD4+/CD8+ T-cell ratio (FIG. 9B), and NKG2D expression (FIG. 9C). FIG. 9A shows flow cytometric analysis of transduction efficiency of the indicated CARs on day 3 and day 10 post gene transfer. Data show median+interquartile range. ****p<0.0001 by Mann-Whitney test. FIG. 9B shows results of CAR T-cells analyzed on day 10 post gene transfer for CD4/CD8 ratio. Data show median+interquartile range. *p<0.05; **p<0.01; ****p<0.0001 by Kruskal-Wallis test. FIG. 9C shows mean fluorescence intensity of NKG2D expression in the indicated CAR T-cells, analyzed on day 10 post gene transfer. Data show median+interquartile range. ***p<0.001; ****p<0.0001 by Kruskal-Wallis test.

FIGS. 10A-10E show results of in vitro assays evaluating cytotoxicity (FIG. 10A), cytokine release (FIGS. 10B-10C), number of productive tumour restimulation cycles (FIG. 10D), and maximum fold T-cell expansion (FIG. 10E) achieved when BXPC3 and Ren tumour cells were co-cultured with adaptor CAR T-cells (NKG2D/Dap10-12; NKG2D/Dap10-CD3ζ; NKG2D/Dap10-CD3ζ (1XX), linear CAR T-cells (NKG2D-CD3ζ), or untransduced T-cells (Untrans). FIG. 10A shows results of MTT cytotoxicity assays in which the indicated CAR T-cells were co-cultivated for 24 hours with BXPC3 or Ren tumour cells at the specified target to effector ratio. Data shown are mean±SEM, ****p<0.0001 by two-way ANOVA. FIG. 10B shows concentration of IL-2 as measured in supernatants collected from tumour cells co-cultured with T-cell populations as indicated after 24 hours incubation (effector:tumour ratio 1:1). Data show median+interquartile range. FIG. 10C shows concentration of IFN-γ as measured in supernatants collected from tumour cells co-cultured with T-cell populations as indicated after 72 hours incubation (effector:tumour ratio 1:1). Data show median+interquartile range. FIG. 10D shows the number of twice weekly re-stimulation cycles achieved by NKG2D adaptor CAR T-cells (NKG2D/Dap10-12 (also, “N/1012”); NKG2D/Dap10-CD3ζ (also, “N/10Z”); NKG2D/Dap10-CD3ζ (1XX) (also, “N/10Z(1XX)”), CAR T-cells (NKG2D-CD3ζ (also, “NZ”)), and control T-cells (NKG2D; Untrans.) on BXPC3 and Ren tumour cells. Data show median+interquartile range. *p<0.01; **p<0.01; ****p<0.0001 by Kruskal Wallis test. FIG. 10E shows the maximum fold expansion of the indicated T-cell population following iterative tumour restimulation. Data show median+interquartile range. *p<0.01; ***p<0.001; ****p<0.0001 by Kruskal Wallis test.

FIGS. 11A-11F show development of subcutaneous BXPC3 tumour xenografts in mice following administration of NKG2D/Dap10-12 adaptor CAR T-cells (FIG. 11A); NKG2D-CD3ζ linear CAR T-cells (FIG. 11B); NKG2D/Dap10-CD3ζ adaptor T-cells (FIG. 11C); NKG2D/Dap10-CD3ζ (1XX) adaptor T-cells (FIG. 11D), PBS (FIG. 11E), or untransduced T-cells (FIG. 11F). BXPC3 cells (1×10⁵ cells) were established subcutaneously in NSG mice for 17 days prior to i.v. administration of 10 million of the indicated T-cell populations, or PBS as control. ****p<0.0001; NS—not significant by two-way ANOVA (post-treatment).

FIG. 12 is a survival curve of the mice assessed for tumour growth in FIGS. 11A-11F.

### 5. DETAILED DESCRIPTION

### 5.1. Definitions

As used herein, the term “variant” refers to a polypeptide sequence which is a naturally occurring polymorphic form of the basic sequence as well as synthetic variants, in which one or more amino acids within the basic sequence are inserted, removed or replaced. However, the variant produces a biological effect which is similar to that of the basic sequence. For example, a variant of the intracellular domain of human CD3 zeta chain will act in a manner similar to that of the intracellular domain of human CD3 zeta chain. Amino acid substitutions may be regarded as “conservative” where an amino acid is replaced with a different amino acid in the same class with broadly similar properties. Non-conservative substitutions are where amino acids are replaced with amino acids of a different type or class.

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation. Non-conservative substitutions may also be possible provided that these do not interrupt the function of the polypeptide as described above. Broadly speaking, fewer non-conservative substitutions will be possible without altering the biological activity of the polypeptides. In general, variants will have amino acid sequences that will be at least 70%, for instance at least 71%, 75%, 79%, 81%, 84%, 87%, 90%, 93%, 95%, 96% or 98% identical to the basic sequence. Identity in this context may be determined using the BLASTP computer program.

As used herein, the term “antigen” refers to any member of a specific binding pair that will bind to the binding elements. The term includes receptors on target cells.

As used herein and with regard to the binding element to a target molecule, the terms “bind,” “specific binding,” “specifically binds to,” “specifically interacts with,” “specific for,” “selectively binds,” “selectively interacts with,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule.

The term “chimeric antigen receptor” or “CAR” as used herein refers to a molecule comprising an extracellular antigen-binding domain that is fused to an intracellular signalling domain that is capable of activating or stimulating an immunoresponsive cell, and a transmembrane domain. In certain embodiments, the extracellular antigen-binding domain of a CAR comprises a scFv.

The term “parallel chimeric antigen receptor” or “pCAR” as used herein refers to a parallel chimeric antigen receptor which comprises the combination of a second generation chimeric antigen receptor (CAR) and, in parallel, a chimeric co-stimulatory receptor (CCR). pCAR has been described in WO2017/021701, which is incorporated by reference in its entirety herein.

The term “adaptor CAR” as used herein refers to an adaptor chimeric antigen receptor which comprises the combination of (i) at least one adaptor protein comprising an activation signalling domain and a co-stimulatory signalling region and (ii) at least one binding domain (targeting moiety) that specifically binds a target antigen. The at least one adaptor protein (i) and the at least one targeting moiety (ii) are expressed as two or more separate polypeptides that associate via non-covalent interaction(s).

The term “linear CAR” as used herein refers to a chimeric antigen receptor which comprises at least one extracellular binding domain (targeting moiety) and one or more intracellular signalling domains in a single polypeptide configured to span a cell's plasma membrane. Examples of linear CARs include, but are not limited to, second-generation CARs.

### 5.2. Other Interpretational Conventions

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### 5.3. Chimeric Antigen Receptors (CARs)

In one aspect, second-generation chimeric antigen receptors (second-generation CARs) are provided. The second-generation CAR comprises (a) an intracellular signalling domain comprising a modified CD3ζ polypeptide; (b) a co-stimulatory signalling region; (c) a transmembrane domain; and (d) a first binding element that specifically interacts with a first epitope on a first target antigen, wherein the modified CD3ζ polypeptide comprises an unmodified ITAM1, and a mutation in ITAM2 and/or ITAM3.

In some embodiments, the second-generation CAR is a single polypeptide.

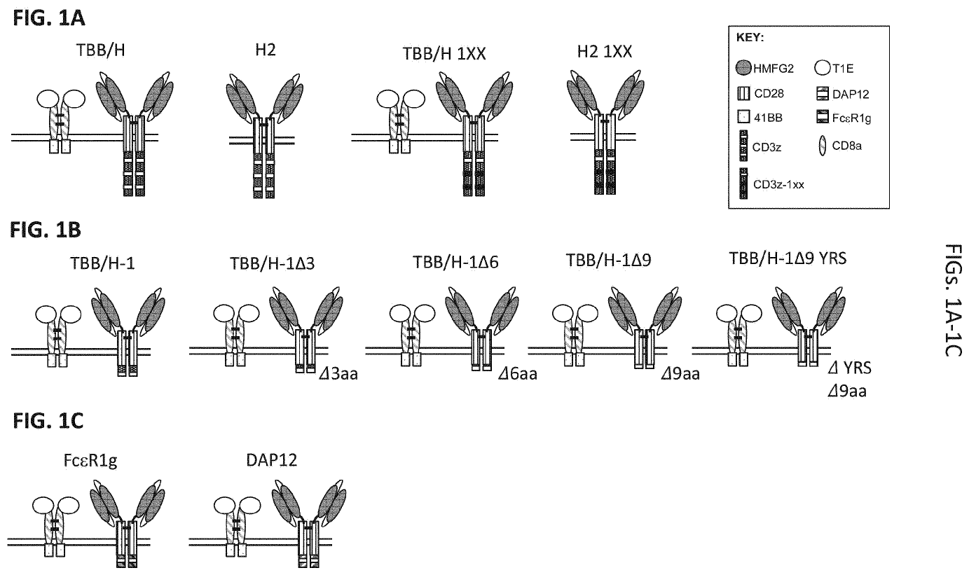

In some embodiments, the CAR is H or H2 as shown in FIG. 1A. H2 CAR is a second-generation (2G) CAR originally described in Wilkie et al., J. Immunol. 180:4901-9 (2008), incorporated herein by reference in its entirety. It comprises, from intracellular to extracellular, a CD3ζ signalling region, CD28 co-stimulatory, transmembrane and extracellular spacer domains, and a human MUC1-targeting HMFG2 scFv domain.

In some embodiments, the CAR is H2 1XX as shown in FIG. 1A. H2 1XX CAR is a second-generation (2G) CAR comprising, from intracellular to extracellular, a modified CD3ζ signalling region, CD28 co-stimulatory, transmembrane and extracellular spacer domains, and a human MUC1-targeting HMFG2 scFv domain.

In some embodiments, the CAR is H2BB CAR. H2BB is a 2G CAR comprising from intracellular to extracellular, a CD3ζ signalling region, a 41BB co-stimulatory domain, a CD8a transmembrane and extracellular spacer domain, and a human MUC1-targeting HMFG2 scFv domain.

### 5.3.1. Intracellular Signalling Domain

The CAR construct comprises a signalling region (i.e. a TCR-like signalling region). In some embodiments, the signalling region comprises an Immune-receptor-Tyrosine-based-Activation-Motif (ITAM), as reviewed for example by Love et al., Cold Spring Harbor Perspect. Biol 2010 2(6)1 a002485. In some embodiments, the signalling region comprises the intracellular domain of human CD3 zeta chain (CD3ζ), as described for example in U.S. Pat. No. 7,446,190, incorporated by reference herein, or a variant thereof. In particular embodiments, the signalling region comprises the domain which spans amino acid residues 52-163 of the full-length human CD3 zeta chain (e.g., SEQ ID NO: 1 or 2). The CD3 zeta chain has a number of known polymorphic forms, (e.g. Sequence ID: gb|AAF34793.1 and gb|AAA60394.1), all of which are useful herein:

(SEQ ID NO: 1)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
(SEQ ID NO: 2)
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

Alternative signalling regions to the CD3 zeta domain include, e.g., FcεR1γ, CD3ε, DAP12, and multi-ITAM. See Eshhar Z et al., Proc Natl Acad Sci USA 90:720-724 (1993); Nolan et al., Clin Cancer Res 5: 3928-3941 (1999); Zhao et al., J Immunol 183: 5563-5574 (2009); Topfer et al., J Immunol 194: 3201-3212 (2015); and James J R, Sci Signal 11(531) eaan1088 (2018), the disclosures of which are incorporated herein by reference in their entireties.

In certain embodiments, the signalling region comprises FcεR1γ endodomain or a variant thereof. In particular embodiments, the FcεR1γ signalling region comprises the amino acid sequence of SEQ ID NO: 20 as shown below:

(SEQ ID NO: 20)
RLKIQVRKAAITSYEKSDGVYTGLSTRNQETYETLKHEKPPQ.

In certain embodiments, the signalling region comprises DAP12 endodomain or a variant thereof. In particular embodiments, the DAP12 signalling region comprises the amino acid sequence of SEQ ID NO: 58.

(SEQ ID NO: 58)
YFLGRLVPRGRGAAEAATRKQRITETESPYQELQGQRSDVYSDLNTQRP
YYK.

### 5.3.1.1 Immunoreceptor Tyrosine-Based Activation Motif (ITAM)

The CAR construct comprises an intracellular signalling domain comprising at least one Immunoreceptor Tyrosine-based Activation Motif (ITAM). In some embodiments, the intracellular signalling domain is TCRζ, FcRγ, FcRβ, CD3γ, CD3θ, CD3δ, CD3ε, CD3ζ, CD22, CD79a, CD79b, and CD66d. In particular embodiments, the intracellular signalling domain is FcεR1γ, DAP12, or CD3ζ. In typical embodiments, the intracellular signalling domain is CD3ζ. The CD3ζ domain has three ITAMs named ITAM1, ITAM2 and ITAM3 from membrane proximal to distal direction.

Primary signalling domains that regulate activation of the TCR complex may contain ITAMs. Non-limiting examples of ITAM-containing intracellular signalling domains include those derived from TCRζ, FcRγ, FcRβ, CD3γ, CD3ε, CD3θ, CD3ε, CD3ζ, CD22, CD79a, CD79b, and CD66d.

### 5.3.1.2 Modified CD3ζ Intracellular Signalling Domains

In some embodiments, the CD3ζ intracellular signalling domain is modified. In some embodiments, the CD3ζ intracellular signalling domain includes modifications in at least one of the three ITAMs. In some embodiments, the CD3ζ intracellular signalling domain is truncated. In some embodiments, the CD3ζ intracellular signalling domain is truncated to remove one or more of the three ITAMs. In some embodiments, the CD3ζ intracellular signalling domain is modified by alternative splicing. In some embodiments, the CD3ζ intracellular signalling domain is engineered to express a fusion polypeptide between CD3ε, CD3θ, or CD30 and CD3ζ.

In various embodiments, at least one, at least two, or all three ITAMs are modified. In some embodiments, at least two of the ITAMs of CD3ζ are modified. In some embodiments, three ITAMs of CD3ζ are modified. In certain embodiments, one ITAM of CD3ζ is unmodified and at least one of the two other two ITAMs is modified.

In certain embodiments, the modified CD3ζ intracellular signalling domain comprises an ITAM1 variant comprising one or more loss-of-function mutations. In certain embodiments, the modified CD3ζ intracellular signalling domain comprises an ITAM1 variant comprising two loss-of-function mutations. In certain embodiments, the loss-of-function mutation comprises a mutation of a tyrosine residue in ITAM1. In certain embodiments, the loss-of-function mutation comprises a mutation of a tyrosine residue to a phenylalanine residue in ITAM1.

In certain embodiments, the modified CD3ζ intracellular signalling domain comprises an ITAM2 variant comprising one or more loss-of-function mutations. In certain embodiments, the modified CD3ζ intracellular signalling domain comprises an ITAM2 variant comprising two loss-of-function mutations. In certain embodiments, the loss-of-function mutation comprises a mutation of a tyrosine residue in ITAM2. In certain embodiments, the loss-of-function mutation comprises a mutation of a tyrosine residue to a phenylalanine residue in ITAM2.

In certain embodiments, the modified CD3ζ intracellular signalling domain comprises an ITAM3 variant comprising one or more loss-of-function mutations. In certain embodiments, the modified CD3ζ intracellular signalling domain comprises an ITAM3 variant comprising two loss-of-function mutations. In certain embodiments, the loss-of-function mutation comprises a mutation of a tyrosine residue in ITAM3. In certain embodiments, the loss-of-function mutation comprises a mutation of a tyrosine residue to a phenylalanine residue in ITAM3.

In certain embodiments, the modified CD3ζ intracellular signalling domain comprises an ITAM2 variant and an ITAM3 variant comprising one or more loss-of-function mutations. In certain embodiments, the modified CD3ζ intracellular signalling domain comprises an ITAM2 variant and an ITAM3 variant comprising two loss-of-function mutations. In certain embodiments, the loss-of-function mutation comprises a mutation of a tyrosine residue in each of ITAM2 and ITAM3. In certain embodiments, the loss-of-function mutation comprises a mutation of a tyrosine residue to a phenylalanine residue in each of ITAM2 and ITAM3.

In certain embodiments, the modified CD3ζ intracellular signalling domain comprises ITAM1 and one or more modifications on at least one of ITAM2 and/or ITAM3. In some embodiments, the modified CD3ζ intracellular signalling domain comprises ITAM1 and one or more loss-of-function mutations in ITAM2 and/or ITAM3. In some embodiments, the modified CD3ζ intracellular signalling domain comprises ITAM1 and two loss-of-function mutations in each of ITAM2 and ITAM3.

In some embodiments, the modified CD3ζ comprises one or more ITAMs comprising two tyrosine (Y) to phenylalanine (F) mutations. In some embodiments, the modified CD3ζ intracellular signalling domain comprises unmodified ITAM1 and two tyrosine (Y) to phenylalanine (F) mutations in ITAM2 and/or ITAM3. In some embodiments, the modified CD3ζ intracellular signalling domain comprises unmodified ITAM1 and two tyrosine (Y) to phenylalanine (F) mutations in each of ITAM2 and ITAM3. In some embodiments, the modified CD3ζ intracellular signalling domain comprises tyrosine (Y) to phenylalanine (F) mutations in at least one, at least two, or all three ITAMs (e.g., ITAM1, ITAM2, and ITAM3).

In various embodiments, the modified CD3ζ polypeptide comprises at least one functional ITAM. In some embodiments, the modified CD3ζ polypeptide comprises mutations in both ITAM2 and ITAM3. In certain embodiments, ITAM2 and ITAM3 in the modified CD3ζ are non-functional. In some embodiments, each of ITAM2 and ITAM3 comprises at least one Tyr to Phe mutation. In typical embodiments, each of ITAM2 and ITAM3 comprises two Tyr to Phe mutations. In certain embodiments, the CD3ζ intracellular signalling domain has the amino acid sequence of SEQ ID NO: 42, SEQ ID NO: 43, or SEQ ID NO: 48 as shown below (ITAM1 bold and single underlined). Residues mutated to phenylalanine (F) from tyrosine (Y) (with reference to unmodified CD3ζ) are double underlined.

(SEQ ID NO: 42)
RVKFSRSADAPAYQQGQNQLYNELNLGRREEYDVLDKRRGRDPEMGGKP
QRRKNPQEGL [symbol] NELQKDKMAEA [symbol] SEIGMKGERRRGKGHDGL [symbol] QGLST
ATKDT [symbol] DALHMQALPPR
(SEQ ID NO: 43)
RVKFSRSAEPPAYQQGQNQLYNELNLGRREEYDVLDKRRGRDPEMGGKP
RRKNPQEGL [symbol] NELQKDKMAEA [symbol] SEIGMKGERRRGKGHDGL [symbol] QGLST
ATKDT [symbol] DALHMQALPPR
(SEQ ID NO: 48)
RVKFSRSADAPAYQQGQNQLYNELNLGRREEYDVLDKRRGRDPEMGGKP
RRKNPQEGL [symbol] NELQKDKMAEA [symbol] SEIGMKGERRRGKGHDGL [symbol] QGLST
ATKDT [symbol] DALHMQALPPR

In some embodiments, the second-generation CAR comprises a truncated CD3ζ intracellular signalling domain. In some embodiments, the CD3ζ is truncated to remove at least one of the three ITAMs (e.g., ITAM1, ITAM2, ITAM3), or combinations thereof. In some embodiments, the CD3ζ is truncated to remove ITAM1. In some embodiments, the CD3ζ is truncated to remove ITAM2. In some embodiments, the CD3ζ is truncated to remove ITAM3. In some embodiments, the CD3ζ is truncated to remove ITAM2 and ITAM3. In certain embodiments, the truncated CD3ζ intracellular signalling domain has the amino acid sequence of SEQ ID NO: 49 as shown below:

(SEQ ID NO: 49)
RVKFSRSADAPAYQQGONQLYNELNLGRREEYDVLDKRRG.

In various embodiments, the modified CD3ζ polypeptide comprises deletion of ITAM2 or a portion thereof and deletion of ITAM3 or a portion thereof. In some embodiments, the modified CD3ζ polypeptide comprises deletion of ITAM3 or a portion thereof and deletion of ITAM2 or a portion thereof. In some embodiments, the modified CD3ζ polypeptide comprises deletion of both ITAM2 and ITAM3. In some embodiments, the modified CD3ζ polypeptide comprises the amino acid sequence having at least 70%, 80%, 90%, 95%, or 99% identity to the N-terminal 40 amino acids of SEQ ID NO: 41 or SEQ ID NO: 48. In some embodiments, the modified CD3ζ polypeptide comprises the N-terminal 40 amino acids of amino acid sequence of SEQ ID NO: 41 or SEQ ID NO: 48.

In some embodiments, the modified CD3ζ polypeptide further comprises deletion of one or more amino acid residues N-terminal to ITAM1. In some embodiments, the modified CD3ζ polypeptide comprises a sequence having at least 70%, 80%, 90%, 95%, or 99% identity to SEQ ID NOs: 50-53. In some embodiments, the modified CD3ζ polypeptide comprises a sequence selected from SEQ ID NOs: 50-53.

One skilled in the art will be capable of introducing mutations into the nucleic acid sequence of a gene or gene product, for example an ITAM, using standard techniques. For example, point mutations can be introduced via site-directed point mutagenesis using PCR.

### 5.3.2. Co-Stimulatory Signalling Region

In the CAR, the co-stimulatory signalling region is suitably located between the signalling region and transmembrane domain, and remote from the binding element.

Suitable co-stimulatory signalling regions are well known in the art, and include the co-stimulatory signalling regions of members of the B7/CD28 family such as B7-1, B7-2, B7-H1, B7-H2, B7-H3, B7-H4, B7-H6, B7-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumour necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha, or TNF RII; or members of the SLAM family such as 2B4, BLAME, CD2, CD2F-10, CD48, CD8, CD84, CD229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP10, DAP12, Dectin-1, DPPIV, EphB6, Integrin alpha 4 beta 1, Integrin alpha 4 beta 7/LPAM-1, LAG-3, TSLP R, lymphocyte function-associated antigen-1 (LFA-1), CD7, NKG2X, CD83 or a variant thereof. See Mondino A et al., J Leukoc Biol. 55:805-815 (1994); Thompson C B, Cell. 81:979-982 (1995); Somoza C et al., Res Immunol. 146:171-176 (1995); Rhodes D A et al., Annu Rev Immunol. 34:151-172 (2016); Foell J et al., Curr Cancer Drug Targets. 7:55-70 (2007); Greenwald R J et al., Annu Rev Immunol. 23:515-548 (2005); Flem-Karlsen K et al., Trends Cancer. 4:401-404 (2018); Flies D B et al., J Immunother. 30:251-260 (2007); Gavrieli M et al., Adv Immunol. 92:157-185 (2006); Zhu Y et al., Nat Commun. 4:2043 (2013); Omar H A et al., Crit Rev Oncol Hematol. 135:21-29 (2019); Hashemi M et al., Oncotarget. 9:24857-24868 (2018); Kang X et al., Cell Cycle. 15:25-40 (2016); Watts T H, Annu Rev Immunol.23:23-68 (2005); Bryceson Y T et al., Immunol Rev. 214:73-91 (2006); Sharpe A H, Curr Opin Immunol. 7:389-395 (1995); Wingren A G et al., Crit Rev Immunol. 15:235-253 (1995), the disclosures of which are incorporated herein by reference in their entireties.

The co-stimulatory signalling regions may be selected depending upon the particular use intended for the immuno-responsive cell. In some embodiments, the co-stimulatory signalling regions are selected from the co-stimulatory signalling regions of CD28, CD27, ICOS, 4-1BB, OX40, Dap10, CD30, GITR, HVEM, DR3 and CD40 or a variant thereof. In certain embodiments, the co-stimulatory signalling regions are selected from the co-stimulatory signalling regions of CD28, 4-1BB, CD27, OX40, Dap10 and ICOS or a variant thereof.

In a particular embodiment, the co-stimulatory signalling region of the CAR is the co-stimulatory signalling region of CD28. In some embodiments, the CD28 co-stimulatory signalling region comprises modification of one or more tyrosine residues in the CD28 cytoplasmic domain. In some embodiments, the CD28 co-stimulatory signalling region comprises a mutation at the C-terminal most tyrosine residue. In some embodiments, the CD28 co-stimulatory signalling region comprises a modified YRS motif. In some embodiments, the CD28 co-stimulatory signalling region comprises a mutation in the YRS motif. In some embodiments, the CD28 co-stimulatory signalling region lacks a YRS motif.

In certain embodiments, the co-stimulatory signalling region of the CAR is the CD28 co-stimulatory signalling region. In certain embodiments, the co-stimulatory signalling region of CD28 comprises the amino acid sequence of SEQ ID NO: 4 as shown below where the endodomain is shown in bold:

(SEQ ID NO: 4)
IEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQP
YAPPRDFAAYRS.

In certain embodiments, the co-stimulatory signalling region of CD28 comprises the amino acid sequence of SEQ ID NO: 25 as shown below:

(SEQ ID NO: 25)
IEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPY
APPRDFAA.

### 5.3.3. Transmembrane Domain

Suitable transmembrane domains are known in the art and include for example, the transmembrane domains of CD8α, CD28, CD4, CD3ζ, FcεR1γ or a variant thereof. Selection of CD3ζ as transmembrane domain may lead to the association of the CAR with other elements of TCR/CD3 complex. This association may recruit more ITAMs but may also lead to the competition between the CAR and the endogenous TCR/CD3.

In certain embodiments, the transmembrane domain of the CAR is selected from the transmembrane domain of CD28 and the transmembrane domain of CD8α. In a particular CAR embodiment, the transmembrane domain of the CAR is the transmembrane domain of CD28.

In some embodiments, the CAR comprises a portion of the extracellular domain and transmembrane domain of CD28.

In certain embodiments, a portion of the CD28 extracellular domain and transmembrane domain comprises the amino acid sequence of SEQ ID NO: 35 as shown below, where the transmembrane domain is shown in bold type:

(SEQ ID NO: 35)
IEVMYPPPYLDNEKSNGTIIHVKGKHLCPSPLFPGPSKPFWVLVVVGG
VLACYSLLVTVAFIIFWV.

### 5.3.4. Co-Stimulatory Signalling Domain and Transmembrane Domain

In some embodiments in which the co-stimulatory signalling region of the CAR is, or comprises, the co-stimulatory signalling region of CD28, the CD28 transmembrane domain represents a suitable, often preferred, option for the transmembrane domain. The full length CD28 protein is a 220 amino acid protein of SEQ ID NO: 3, where the transmembrane domain is shown in bold type:

(SEQ ID NO: 3)
MLRLLLALNLFPSIQVTGNKILVKQSPMLVAYDNAVNLSCKYSYNLFS
REFRASLHKGLDSAVEVCVVYGNYSQQLQVYSKTGFNCDGKLGNESVT
FYLQNLYVNQTDIYFCKIEVMYPPPYLDNEKSNGTIIHVKGKHLCPSP
LFPGPSKPFWVLVVVGGVLACYSLLVTVAFIIFWVRSKRSRLLHSDYM
NMTPRRPGPTRKHYQPYAPPRDFAAYRS.

In some embodiments, one of the co-stimulatory signalling regions is based upon the hinge region and suitably also the transmembrane domain and endodomain of CD28. In some embodiments, the co-stimulatory signalling region comprises amino acids 114-220 of SEQ ID NO: 3, shown below as SEQ ID NO: 4, where the transmembrane domain is shown in bold type:

(SEQ ID NO: 4)
IEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPY
APPRDFAAYRS.

In some embodiments in which the co-stimulatory signalling region of the CAR is, or comprises, the co-stimulatory signalling region of Dap10, the Dap10 transmembrane domain represents a suitable, often preferred, option for the transmembrane domain. The full length Dap10 protein is a 93 amino acid protein of SEQ ID NO: 47, where the transmembrane domain is shown in bold type:

(SEQ ID NO: 47)
MIHLGHILFLLLLPVAAAQTTPGERSSLPAFYPGTSGSCSGCGSLSLP
LLAGLVAADAVASLLIVGAVFLCARPRRSPAQEDGKVYINMPGRG

### 5.3.5. CAR Binding Element

The binding element of the CAR binds at least one epitope.

In various embodiments, the binding element of the CAR specifically binds to an epitope.

In various embodiments, the binding element of the CAR binds to a first target antigen. In certain embodiments, the at least one antigen are associated with a disease, such as a cancer.

Thus, a suitable binding element may be any element which provides the CAR with the ability to recognize a target of interest. The target to which the CAR of the disclosure directed can be any target of clinical interest to which it would be desirable to direct a T cell response.

In various embodiments, the binding element used in the CARs described herein are antigen binding sites (ABS) of antibodies. In typical embodiments, the ABS used as the binding element is formatted into an scFv or is single domain antibody from a camelid, human, or other species.

Alternatively, a binding element of a CAR may comprise ligands that bind to a surface protein of interest.

In some embodiments, the binding element is associated with a leader sequence which facilitates expression on the cell surface. Many leader sequences are known in the art, and these include the macrophage colony-stimulating factor receptor (FMS) leader sequence or CD124 leader sequence.

In some embodiments, the binding element specifically binds a tumour antigen or tumour-associated antigen, providing a means to target tumour cells while limiting damage to non-tumour cells or tissues.

In some embodiments, the binding element specifically binds a first epitope on a first target antigen selected from an NKG2D ligand (e.g., MICA, MICB, ULBP1-ULBP6), MUC1, αvβ6 integrin, ErbB1-4, HER2, B7-H3, Claudin 18.2, Claudin 6, Glypican 3, ALK, CD70, GD2 and prostate-specific membrane antigen (PSMA).

In some embodiments, the binding element specifically binds an epitope on the antigen MUC1.

### 5.3.5.1 MUC1 CAR

In particular embodiments, the binding element specifically interacts with an epitope on a MUC1 target antigen.

In currently preferred embodiments, the binding element of the CAR specifically interacts with a first epitope on a MUC1 target antigen. In some embodiments, the CAR binding element comprises the antigen binding site of an antibody specific to MUC1. In some embodiments, the CAR binding element comprises CDRs of an antibody specific to MUC1. In some embodiments, the CAR binding element comprises V_{H} and V_{L} sequences of an antibody specific to MUC1.

In some embodiments, the CAR binding element comprises the antigen binding site of the HMFG2 antibody. In certain embodiments, the CAR binding element comprises the CDRs of the HMFG2 antibody. The CDR sequences of the HMFG2 antibody were determined using the tools provided on www.abysis.org and are shown below as SEQ ID NOs: 8-13:

V_{H} CDR1
(SEQ ID NO: 8)
GFTFSNY;
V_{H} CDR2
(SEQ ID NO: 9)
RLKSNNYA;
V_{H} CDR3
(SEQ ID NO: 10)
GNSFAY;
V_{L} CDR1
(SEQ ID NO: 11)
RSSTGAVTTSNYAN;
V_{L} CDR2
(SEQ ID NO: 12)
GTNNRAP;
V_{L} CDR3
(SEQ ID NO: 13)
ALWYSNHWV.

In certain embodiments, the CAR binding element comprises the V_{H} and V_{L} domains of the HMFG2 antibody. The V_{H} and V_{L} domain sequences of the HMFG2 antibody are shown below as SEQ ID NOs: 14-15:

(SEQ ID NO: 14)
EVQLQQSGGGLVQPGGSMKLSCVASGFTFSNYWMNWVRQSPEKGLEWVA
EIRLKSNNYATHYAESVKGRFTISRDDSKSSVYLQMNNLRAEDTGIYYC
TFGNSFAYWGQGTTVTVSS;
(SEQ ID NO: 15)
QAVVTQESALTTSPGETVTLTCRSSTGAVTTSNYANWVQEKPDHLFTGL
IGGTNNRAPGVPARFSGSLIGDKAALTITGAQTEDEAIYFCALWYSNHW
VFGGGTKLTVLGSE.

In particularly preferred embodiments, the CAR binding element comprises the antigen binding site of the HMFG2 antibody formatted as a scFv. In certain embodiments, the amino acid sequence of the scFv of the HMGF2 antibody is 70%, 75%, 80%, 85%, 90%, 95%, 97%, 99%, or 100% identical to SEQ ID NO: 16 shown below:

(SEQ ID NO: 16)
EVQLQQSGGGLVQPGGSMKLSCVASGFTFSNYWMNWVRQSPEKGLEWVA
EIRLKSNNYATHYAESVKGRFTISRDDSKSSVYLQMNNLRAEDTGIYYC
TFGNSFAYWGQGTTVTVSSGGGGSGGGGSGGGGSQAVVTQESALTTSPG
ETVTLTCRSSTGAVTTSNYANWVQEKPDHLFTGLIGGTNNRAPGVPARF
SGSLIGDKAALTITGAQTEDEAIYFCALWYSNHWVFGGGTKLTVLGSE.

In certain embodiments, the nucleic acid encoding the scFv of the HMGF2 antibody is SEQ ID NO: 17 shown below:

(SEQ ID NO: 17)
GAGGTGCAGCTGCAGCAGTCTGGAGGAGGCTTGGTGCAACCTGGAGGAT
CCATGAAACTCTCCTGTGTTGCCTCTGGATTCACTTTCAGTAACTACTG
GATGAACTGGGTCCGCCAGTCTCCAGAGAAGGGGCTTGAGTGGGTTGCT
GAAATTAGATTGAAATCTAATAATTATGCAACACATTATGCGGAGTCTG
TGAAAGGGAGGTTCACCATCTCAAGAGATGATTCCAAAAGTAGTGTCTA
CCTGCAAATGAACAACTTAAGAGCTGAAGACACTGGCATTTATTACTGT
ACCTTTGGTAACTCCTTTGCTTACTGGGGCCAAGGGACCACGGTCACCG
TCTCCTCAGGTGGAGGCGGTTCAGGCGGAGGTGGCTCTGGCGGTGGCGG
ATCGCAGGCCGTGGTCACTCAGGAATCTGCACTCACCACATCACCTGGT
GAAACAGTCACACTCACTTGTCGCTCAAGTACTGGGGCTGTTACAACTA
GTAACTATGCCAACTGGGTCCAAGAAAAACCAGATCATTTATTCACTGG
TCTAATAGGTGGTACCAACAACCGAGCACCAGGTGTTCCTGCCAGATTC
TCAGGCTCCCTGATTGGAGACAAGGCTGCCCTCACCATCACAGGGGCAC
AGACTGAGGATGAGGCAATATATTTCTGTGCTCTATGGTACAGCAACCA
TTGGGTGTTCGGTGGAGGAACCAAACTGACTGTCCTAGGATCAGAG.

### 5.3.5.2 NKG2DL CAR

In particular embodiments, the binding element specifically interacts with an epitope on an NKG2D receptor ligand.

In some embodiments, the CAR binding element comprises human NKG2D receptor polypeptide. In some embodiments, the CAR binding element comprises a fragment, portion, or variant of human NKG2D receptor polypeptide.

The NKG2D-CD3ζ linear CAR (SEQ ID NO: 69) comprises an NKG2D ligand binding domain (SEQ ID NO: 75) fused to a CD3ζ polypeptide (SEQ ID NO: 1).

The protein sequence of the NKG2D-CD3ζ linear CAR is shown below as SEQ ID NO: 69. CD3ζ sequence is italicized and ITAMs of CD3ζ are in bold; NKG2D sequence is double underlined.

(SEQ ID NO: 69)
MRVKFSRSADAPAYQQGQNQLYNELNLGRREEYDVLDKRRGRDPEMGG
KPRRKNPQEGLYNELQKDKMAEAYSEIGMKGERRRGKGHDGLYQGLST
ATKDTYDALHMQALPPRGWIRGRRSRHSWEMSEFHNYNLDLKKSDFST
RWQKQRCPVVKSKCRENASPFFFCCFIAVAMGIRFIIMVAIWSAVFLN
SLFNQEVQIPLTESYCGPCPKNWICYKNNCYQFFDESKNWYESQASCM
SQNASLLKVYSKEDQDLLKLVKSYHWMGLVHIPTNGSWQWEDGSILSP
NLLTIIEMQKGDCALYASSFKGYIENCSTPNTYICMQRTV.

5.4. Parallel Chimeric Antigen Receptors (pCARs)

In one aspect, parallel chimeric antigen receptors (pCARs) are provided. The pCAR comprises a second-generation CAR and a chimeric co-stimulatory receptor (CCR). The pCAR comprises a second-generation CAR comprising (a) an intracellular signalling domain comprising a CD3ζ polypeptide; (b) a co-stimulatory signalling region; (c) a transmembrane domain; and (d) a first binding element that specifically interacts with a first epitope on a first target antigen, wherein the modified CD3ζ polypeptide comprises an unmodified immunoreceptor tyrosine-based activation motif 1 (ITAM1), and a mutation in ITAM2 and/or ITAM3, and a CCR comprising (e) a co-stimulatory signalling region which is different from that of (b); (f) a transmembrane domain; and (g) a second binding element that specifically interacts with a second epitope on a second target antigen.

In some embodiments, the pCAR is a TBB/H pCAR as shown in FIG. 1A. The TBB/H pCARs utilize the MUC1-targeting second-generation “H2” CAR and a co-expressed chimeric co-stimulatory receptor (CCR). The CCR in the TBB/H pCAR has a T1E binding domain fused to CD8a transmembrane domain and a 4-1BB co-stimulatory domain. T1E is a chimeric peptide derived from transforming growth factor-α (TGF-α) and epidermal growth factor (EGF) and is a promiscuous ErbB ligand. See Wingens et al., J. Biol. Chem. 278:39114-23 (2003) and Davies et al., Mol. Med. 18:565-576 (2012), the disclosures of which are incorporated herein by reference in their entireties. Unlike the CAR, the CCR lacks a TCR-like signalling region such as CD3ζ. Parallel CAR (pCAR)-engineered T cells demonstrate superior activity and resistance to exhaustion as compared to first generation CAR-T cells, second-generation CAR-T cells, and third generation CAR-T cells. See PCT Publication WO2020183158A1, incorporated herein by reference in its entirety.

In some embodiments, the pCAR is a TBB/H 1XX pCAR as shown in FIG. 1A. The TBB/H 1XX pCAR is the same as the TBB/H pCAR described above except that the CD3ζ polypeptide is modified to comprise one or more mutations in ITAM2 and ITAM3, as described in 5.3.1.2 above.

In some embodiments, the pCAR is a TBB/H 1XX pCAR variant as shown in FIG. 1B. In some embodiments, the TBB/H 1XX pCAR variant is TBB/H-1, which does not contain the two distal ITAMs (ITAM2 and ITAM3) of CD3ζ. In some embodiments, the TBB/H 1XX pCAR variant comprises a proximal ITAM (ITAM1) and one or more tyrosine (Y) to phenylalanine (F) mutations on each of ITAM2 and ITAM3. In some embodiments, the TBB/H 1XX pCAR comprises a proximal ITAM (ITAM1) and two tyrosine (Y) to phenylalanine (F) mutations on each of ITAM2 and ITAM3.

In some embodiments, the pCAR is a variant of the TBB/H-1 pCAR and lacks one or more amino acids upstream of the functional ITAM (ITAM1) of CD3ζ when compared to the TBB/H-1 pCAR. In some embodiments, the TBB/H-1 pCAR variant lacks 3 amino acid residues upstream of the functional ITAM (ITAM1) of CD3ζ when compared to the TBB/H-1 pCAR (TBB/H-1Δ3). In some embodiments, the TBB/H-1 pCAR variant lacks 6 amino acid residues upstream of the functional ITAM (ITAM1) of CD3ζ when compared to the TBB/H-1 pCAR (TBB/H-1Δ6). In some embodiments, the TBB/H-1 pCAR variant lacks 9 amino acid residues upstream of the functional ITAM (ITAM1) of CD3ζ when compared to the TBB/H-1 pCAR (TBB/H-1Δ9).

In some embodiments, the TBB/H-1 pCAR variant lacks 9 amino acid residues upstream of the functional ITAM (ITAM1) of CD3ζ and further lacks the YRS motif of CD28 (i.e. TBB/H-1Δ9 YRS).

In some embodiments, the pCAR is a TBB/H-FcεR1γ pCAR or TBB/H-DAP12 pCAR as shown in FIG. 1C. In such embodiments, the TBB/H-FcεR1γ pCAR comprises a FcεR1γ alternative activation domain substituting the CD3ζ domain. In such embodiments, the TBB/H-DAP12 pCAR comprises a DAP12 alternative activation domain substituting the CD3ζ domain.

### 5.4.1. Chimeric Co-Stimulatory Receptor (CCR)

The chimeric co-stimulatory receptor (CCR) comprises, from C-terminus to N-terminus (from intracellular to extracellular as expressed within the immuno-responsive cell), (e) at least one second co-stimulatory signalling region which is different from that of the first co-stimulatory signalling region of the CAR; (f) at least one second transmembrane domain; and (g) at least one second binding element that specifically binds a second epitope on a second target antigen.

In some embodiments, the CCR is a single polypeptide. In some embodiments, the CCR is two polypeptides. In some embodiments, the CCR is three polypeptides. In some embodiments, the CCR is more than three polypeptides.

In some embodiments, the second binding element specifically binds a second epitope on a second target antigen selected from ErbB1-4, HER2, CD19, B7-H3, GD2, αvβ6 integrin, NKG2D ligand (e.g., MICA, MICB, ULBP1-ULBP6), NKp30 ligand (e.g., BAG6, B7-H6), NKp44 ligand (e.g., NKp44L (MLL5), PCNA, viral haemagglutinins, nidogen-1, galectin-3, other proteoglycans), NKp46 ligand, MUC1, Claudin 18.2, Claudin 6, Glypican 3, ALK, CD70, GD2, PD-L1 and PSMA or a variant thereof.

The second epitope can be identical to or distinct from the first epitope.

In some embodiments, pCARs comprise a CCR comprising two CCR polypeptides that dimerise upon ligand binding. In some embodiments, pCARs comprise a CCR with three CCR polypeptides that trimerize upon ligand binding.

In some embodiments, the CCR is monomeric. In some embodiments, the CCR is dimeric. In some embodiments, the CCR is trimeric.

In some embodiments, the dimerized CCR comprises a second binding element that specifically binds an epitope on an ErbB antigen. In some embodiments, the second binding element is a T1E peptide. In some embodiments, the second binding element has the amino acid sequence of SEQ ID NO: 18.

In some embodiments, the dimerized CCR comprises a second binding element that specifically binds an NKG2D ligand (e.g., MICA, MICB, ULBP1-ULBP6). In some embodiments, the second binding element is an NKG2D polypeptide. In some embodiments, the second binding element has the amino acid sequence of SEQ ID NO: 75. In some embodiments, the dimerized CCR has the amino acid sequence of SEQ ID NO: 77.

In some embodiments, the trimerized CCR comprises a second binding element that specifically binds an NKG2D ligand (e.g., MICA, MICB, ULBP1-ULBP6). In some embodiments, the second binding element is an NKG2D polypeptide. In some embodiments, the second binding element has the amino acid sequence of SEQ ID NO: 75. In some embodiments, the trimerized CCR has the amino acid sequence of SEQ ID NO: 79.

In some embodiments, the trimerized CCR comprises a second binding element that specifically binds a PD-1 ligand. In some embodiments, the second binding element is a PD-1 polypeptide. In some embodiments, the second binding element has the amino acid sequence of SEQ ID NO: 86. In some embodiments, the trimerized CCR has the amino acid sequence of SEQ ID NO: 81.

In some embodiments, the trimerized CCR comprises a second binding element that specifically binds an NKp44 ligand. In some embodiments, the second binding element is an NKp44 polypeptide. In some embodiments, the second binding element has the amino acid sequence of SEQ ID NO: 87. In some embodiments, the trimerized CCR has the amino acid sequence of SEQ ID NO: 83.

### 5.4.1.1 Co-Stimulatory Signalling Region

In the CCR, the co-stimulatory signalling region is suitably located adjacent the transmembrane domain and remote from the binding element.

Suitable co-stimulatory signalling regions are well known in the art, and include the co-stimulatory signalling regions of members of the B7/CD28 family such as B7-1, B7-2, B7-H1, B7-H2, B7-H3, B7-H4, B7-H6, B7-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumour necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha, or TNF RII; or members of the SLAM family such as 2B4, BLAME, CD2, CD2F-10, CD48, CD8, CD84, CD229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP10, DAP12, Dectin-1, DPPIV, EphB6, Integrin alpha 4 beta 1, Integrin alpha 4 beta 7/LPAM-1, LAG-3, TSLP R, lymphocyte function-associated antigen-1 (LFA-1), CD7, NKG2X, CD83 or a variant thereof. See Mondino A et al., J Leukoc Biol. 55:805-815 (1994); Thompson C B, Cell. 81:979-982 (1995); Somoza C et al., Res Immunol. 146:171-176 (1995); Rhodes D A et al., Annu Rev Immunol. 34:151-172 (2016); Foell J et al., Curr Cancer Drug Targets. 7:55-70 (2007); Greenwald R J et al., Annu Rev Immunol. 23:515-548 (2005); Flem-Karlsen K et al., Trends Cancer. 4:401-404 (2018); Flies D B et al., J Immunother. 30:251-260 (2007); Gavrieli M et al., Adv Immunol. 92:157-185 (2006); Zhu Y et al., Nat Commun. 4:2043 (2013); Omar H A et al., Crit Rev Oncol Hematol. 135:21-29 (2019); Hashemi M et al., Oncotarget. 9:24857-24868 (2018); Kang X et al., Cell Cycle. 15:25-40 (2016); Watts T H, Annu Rev Immunol.23:23-68 (2005); Bryceson Y T et al., Immunol Rev. 214:73-91 (2006); Sharpe A H, Curr Opin Immunol. 7:389-395 (1995); Wingren A G et al., Crit Rev Immunol. 15:235-253 (1995), the disclosures of which are incorporated herein by reference in their entireties.

The co-stimulatory signalling regions may be selected depending upon the particular use intended for the immuno-responsive cell. In particular, the co-stimulatory signalling region of the CCR can be selected to work additively or synergistically together with the co-stimulatory signalling region of the CAR. In some embodiments, the co-stimulatory signalling region of the CCR is selected from the co-stimulatory signalling regions of CD28, CD27, ICOS, 4-1BB, OX40, CD30, GITR, HVEM, DR3, Dap10 and CD40 or a variant thereof.

In some embodiments, the co-stimulatory signalling region of the CCR is selected from a 4-1BB co-stimulatory domain, a CD28 co-stimulatory domain, an OX40 co-stimulatory domain, and an ICOS co-stimulatory domain. In certain embodiments, the co-stimulatory signalling region of the CCR has the amino acid sequence of any one of SEQ ID NO: 4, SEQ ID NO: 25, SEQ ID NO: 37, SEQ ID NO: 38, SEQ ID NO: 39, and SEQ ID NO: 40.

In some embodiments, the co-stimulatory signalling region of the CCR is a 4-1BB co-stimulatory domain.

In certain embodiments, one co-stimulatory signalling region of the pCAR is the co-stimulatory signalling region of CD28 and the other is the co-stimulatory signalling region of 4-1BB. In certain embodiments, one co-stimulatory signalling region of the pCAR is the co-stimulatory signalling region of CD28 and the other is the co-stimulatory signalling region of CD27. In certain embodiments, one co-stimulatory signalling region of the pCAR is the co-stimulatory signalling region of CD28 and the other is the co-stimulatory signalling region of OX40. In certain embodiments, one co-stimulatory signalling region of the pCAR is the co-stimulatory signalling region of ICOS and the other is the co-stimulatory signalling region of 4-1BB.

In a particular pCAR embodiment, the co-stimulatory signalling region of the CAR is the co-stimulatory signalling region of CD28 and the co-stimulatory signalling region of the CCR is the co-stimulatory signalling region of 4-1BB.

In certain embodiments, the co-stimulatory signalling region of the CCR is the co-stimulatory signalling region of 4-1BB. In certain embodiments, the co-stimulatory signalling region of 4-1BB comprises the amino acid sequence of SEQ ID NO: 37 as shown below:

(SEQ ID NO: 37)
KRGRKKLLYIFKQPFMRPVQTTQEEDGCSCRFPEEEEGGCEL.

### 5.4.1.2 Transmembrane Domain

The transmembrane domains for the CAR and CCR constructs may be the same or different. In currently preferred embodiments, when the CAR and CCR constructs are expressed from a single vector, the transmembrane domains of the CAR and CCR are different, to ensure separation of the constructs on the surface of the cell. Selection of different transmembrane domains may also enhance stability of the expression vector since inclusion of a direct repeat nucleic acid sequence in the viral vector renders it prone to rearrangement, with deletion of sequences between the direct repeats. In embodiments in which the transmembrane domains of the CAR and CCR of the pCAR are chosen to be the same, this risk can be reduced by modifying or “wobbling” the codons selected to encode the same protein sequence.

Suitable transmembrane domains are known in the art and include for example, the transmembrane domains of CD8α, CD28, CD4, CD3ζ, FcεR1γ or a variant thereof. Selection of CD3ζ as transmembrane domain may lead to the association of the CAR with other elements of TCR/CD3 complex. This association may recruit more ITAMs but may also lead to the competition between the CAR and the endogenous TCR/CD3.

In certain embodiments, the transmembrane domain of the CCR is the transmembrane domain of CD8α.

In some embodiments, the CCR comprises a portion of the extracellular domain and transmembrane domain of CD8α.

In certain embodiments, a portion of the CD8a extracellular domain and transmembrane domain comprises the amino acid sequence of SEQ ID NO: 36 as shown below:

(SEQ ID NO: 36)
PTTTPAPRPPTPAPTIASQPLSLRPEACRPAAGGAVHTRGLDFACDIYI
WAPLAGTCGVLLLSLVITLYCNH

### 5.4.1.3 CCR Binding Element

The binding elements of the CAR and CCR constructs of the pCAR respectively bind a first epitope and a second epitope.

In typical embodiments, the binding elements of the CAR and CCR constructs are different from one another.

In various embodiments, the binding elements of the CAR and CCR specifically bind to a first epitope and second epitope of the same antigen. In certain of these embodiments, the binding elements of the CAR and CCR specifically bind to the same, overlapping, or different epitopes of the same antigen. In embodiments in which the first and second epitopes are the same or overlapping, the binding elements on the CAR and CCR can compete in their binding.

In various embodiments, the binding elements of the CAR and CCR constructs of the pCAR bind to different antigens. In certain embodiments, the antigens are different but may be associated with the same disease, such as the same specific cancer.

Thus, a suitable binding element may be any element which provides the CCR with the ability to recognize a target of interest. The target to which the CCRs of the disclosure are directed can be any target of clinical interest to which it would be desirable to direct a T cell response.

In various embodiments, the binding elements used in CCRs of the pCARs described herein are antigen binding sites (ABS) of antibodies. In typical embodiments, the ABS used as the binding element is formatted into a single chain antibody (scFv) or is single domain antibody from a camelid, human or other species.

Alternatively, a binding element of a CCR of a pCAR disclosed herein may comprise ligands that bind to a surface protein of interest.

In some embodiments, the binding element is associated with a leader sequence which facilitates expression on the cell surface. Many leader sequences are known in the art, and these include the macrophage colony-stimulating factor receptor (FMS) leader sequence or CD124 leader sequence.

In some embodiments, the binding element specifically binds a tumour antigen or tumour-associated antigen, providing a means to target tumour cells while limiting damage to non-tumour cells or tissues.

In some embodiments, the binding element of the CCR specifically binds an epitope on a second target antigen selected NKG2D ligand (e.g., MICA, MICB, ULBP1-ULBP6), MUC1, αvβ6 integrin, ErbB, HER2, B7-H3, Claudin 18.2, Claudin 6, Glypican 3, ALK, CD70, and PSMA, PD-L1, and NKp30 ligands (e.g., BAG6 and B7-H6).

In some embodiments, the binding element of the CCR specifically binds an epitope on a NKG2D ligand (e.g., MICA, MICA, MICB, ULBP1, ULBP2, ULBP3, ULBP4, ULBP5, or ULBP6). In some embodiments, the binding element of the CCR is a NKG2D polypeptide or a fragment or functional variant thereof a.

In some embodiments, the binding element of the CCR specifically binds an epitope on a PD-L1 ligand (e.g., PD-L1). In some embodiments, the binding element of the CCR is a PD-1 polypeptide or a fragment or functional variant thereof.

In some embodiments, the binding element of the CCR specifically binds an epitope on a NKp44 ligand. In some embodiments, the binding element of the CCR is a NKp44 polypeptide or a fragment or functional variant thereof.

In some embodiments, the binding element of the CCR specifically interacts with an epitope on an ErbB receptor. In some embodiments, the binding element of the CCR is a T1E peptide, an antigen binding site of HER2, or an antigen binding site of another ErbB family member. In some embodiments, the binding element of the CCR is a T1E peptide.

In some embodiments, the binding element of the CCR is the T1E peptide, which binds ErbB homo- and hetero-dimers. T1E is a chimeric peptide derived from transforming growth factor-α (TGF-α) and epidermal growth factor (EGF) and is a promiscuous ErbB ligand. The T1E peptide is a chimeric fusion protein composed of the entire mature human EGF protein, excluding the five most N-terminal amino acids (amino acids 971-975 of pro-epidermal growth factor precursor (NP 001954.2)), which have been replaced by the seven most N-terminal amino acids of the mature human TGF-α protein (amino acids 40-46 of pro-transforming growth factor alpha isoform 1 (NP 003227.1)). See Wingens et al., J. Biol. Chem. 278:39114-23 (2003) and Davies et al., Mol. Med. 18:565-576 (2012), the disclosures of which are incorporate herein by reference in their entireties. The sequence of T1E is shown below as SEQ ID NO: 18:

(SEQ ID NO: 18)
VVSHFNDCPLSHDGYCLHDGVCMYIEALDKYACNCVVGYIGERCQYRDL
KWWELR.

In some embodiments, the binding element of the CCR is ICR62, which binds to EGFR. In particular embodiments, the binding element of the CCR comprises the antigen binding site of the ICR62 antibody formatted as scFv. In certain embodiments, the amino acid sequence of the scFv of the ICR62 antibody is 70%, 75%, 80%, 85%, 90%, 95%, 97%, 99%, or 100% identical to SEQ ID NO: 44 shown below:

(SEQ ID NO: 44)
QVNLLQSGAALVKPGASVKLSCKGSGFTFTDYKIHWVKQSHGKSLEWIG
YFNPNSGYSTYNEKFKSKATLTADKSTDTAYMELTSLTSEDSATYYCTR
LSPGGYYVMDAWGQGASVTVSSAQTTAPSVYPLAPGSGGGGSGGGGSGG
GGSDIQMTQSPSFLSASVGDRVTINCKASQNINNYLNWYQQKLGEAPKR
LIYNTNNLQTGIPSRFSGSGSGTDYTLTISSLQPEDFATYFCLQHNSFP
TFGAGTKLELKRADAAPTVSIFPPSKS.

In some embodiments, the binding element of the CCR specifically binds PD-L1.

In some embodiments, the binding element specifically binds an NKp30 ligand selected from BAG6 and B7-H6.

In some embodiments, the second-generation CAR has at least 90% sequence identity to the amino acid sequence of SEQ ID NO: 59 and the CCR has at least 90% sequence identity to the amino acid sequence of SEQ ID NO:24.

5.4.2. Exemplary pCARs

The TBB/H pCAR (SEQ ID NO: 7) comprises: (i) a CCR comprising a T1E binding domain fused to a portion of the CD8a ectodomain, followed by a CD8α transmembrane domain and a 4-1BB co-stimulatory domain (“TBB”) (SEQ ID NO: 24) and (ii) a second-generation CAR comprising a human MUC1-targeting HMFG2 scFv fused to a portion of the CD28 ectodomain, followed by a CD28 transmembrane domain, a CD28 co-stimulatory domain, and a CD3ζ signalling region (“H2” (SEQ ID NO: 21)). The CCR and the CAR are linked by a furin cleavage site (RRKR (SEQ ID NO: 31)), Ser-Gly linker (SGSG (SEQ ID NO: 32)), and T2A ribosomal skip peptide (EGRGSLLTCGDVEENPGP (SEQ ID NO: 33)).

The protein sequence of the TBB/H pCAR is shown below as SEQ ID NO: 7. The V_{H} and the V_{L} sequences of HMFG2 are underlined and in bold, the T1E peptide sequence is italicized, underlined and in bold, and the sequence of the CD3ζ signalling region is italicized with the ITAM sequences in bold and italics:

(SEQ ID NO: 7)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTA
TKDTYDALHMQALPPR.

The protein sequence of the CCR (“TBB”) of TBB/H is shown below as SEQ ID NO: 24 with the T1E peptide underlined and highlighted in bold:

(SEQ ID NO: 24)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCEL.

The protein sequence of the CAR (“H2”) of TBB/H is shown below as SEQ ID NO: 21 with the V_{H} and the V_{L} sequences of HMFG2 underlined and in bold and the ITAMs of the CD3ζ polypeptide are italicized and in bold:

(SEQ ID NO: 21)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR.

The TBB/H 1XX pCAR (SEQ ID NO: 23) comprises (i) a CCR comprising a T1E binding domain fused to a portion of the CD8α ectodomain, followed by a CD8α transmembrane domain and a 4-1BB co-stimulatory domain (“TBB”) (SEQ ID NO: 24) and (ii) a second-generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H2 1XX”) (SEQ ID NO: 59). The modified CD3ζ polypeptide (SEQ ID NO: 48) comprises a wild-type immunoreceptor tyrosine-based activation motif 1 (ITAM1) and two tyrosine to phenylalanine mutations in each of ITAM2 and ITAM3. The CCR and the CAR are linked by a furin cleavage site (RRKR (SEQ ID NO: 31)), Ser-Gly linker (SGSG (SEQ ID NO: 32)), and T2A ribosomal skip peptide (EGRGSLLTCGDVEENPGP (SEQ ID NO: 33)).

The protein sequence of TBB/H 1XX pCAR is shown below as SEQ ID NO: 23. The V_{H} and V_{L} sequences of HMFG2 are underlined and in bold and the ITAMs of the CD3ζ polypeptide are italicized and in bold. Residues mutated to phenylalanine (F) from tyrosine (Y) (with reference to unmodified CD3ζ) are double underlined.

(SEQ ID NO: 23)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRVKFSRSADAPAYQQGQNQLYNELNLGRREEYDVLDKRRGRDPEMGG
KPRRKNPQEGLFNELQKDKMAEAFSEIGMKGERRRGKGHDGLFQGLSTA
TKDTFDALHMQALPPR.

The protein sequence of the CCR (“TBB”) of TBB/H 1XX is shown above as SEQ ID NO: 24 with the T1E peptide underlined and highlighted in bold.

The protein sequence of the CAR (“H 1XX”) of TBB/H 1XX is shown below as SEQ ID NO: 59 with the V_{H} and V_{L} sequences of HMFG2 are underlined and in bold, the ITAMs of CD3ζ are italicized and in bold.

(SEQ ID NO: 59)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQNQL
YNELNLGRREEYDVLDKRRGRDPEMGGKPRRKNPQEGLFNELQKDKMAE
AFSEIGMKGERRRGKGHDGLFQGLSTATKDTFDALHMQALPPR

The TBB/H-1 pCAR (SEQ ID NO: 26) comprises (i) a CCR comprising a TIE binding domain fused to a portion of the CD8α ectodomain, followed by a CD8α transmembrane domain and a 4-1BB co-stimulatory domain (“TBB”) (SEQ ID NO: 24) and (ii) a second-generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H-1”) (SEQ ID NO: 22). The modified CD3ζ polypeptide comprises only one immunoreceptor tyrosine-based activation motif 1 (ITAM1), with each of ITAM2 and ITAM3 deleted from the CD3ζ polypeptide. The CCR and the CAR are linked by a furin cleavage site (RRKR (SEQ ID NO: 31)), Ser-Gly linker (SGSG (SEQ ID NO: 32)), and T2A ribosomal skip peptide (EGRGSLLTCGDVEENPGP (SEQ ID NO: 33)).

The protein sequence of TBB/H-1 pCAR is shown below as SEQ ID NO: 26. The V_{H} and V_{L} sequences of HMFG2 are underlined and in bold, ITAM1 of the CD3ζ polypeptide is italicized and in bold.

(SEQ ID NO: 26)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRVKFSRSADAPAYQQGQNQLYNELNLGRREEYDVLDKRRG.

The protein sequence of the CCR (“TBB”) of TBB/H-1 is shown above as SEQ ID NO: 24 with the T1E peptide underlined and highlighted in bold.

The protein sequence of the CAR (“H-1”) of TBB/H-1 is shown below as SEQ ID NO: 22 with V_{H} and V_{L} sequences of HMFG2 and ITAM1 of the CD3ζ polypeptide in italics and bold:

(SEQ ID NO: 22)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQNQL
YNELNLGRREEYDVLDKRRG.

The various TBB/H-1Δ pCARs comprise (i) a CCR comprising a T1E binding domain fused to a portion of the CD8α ectodomain, followed by a CD8α transmembrane domain and a 4-1BB co-stimulatory domain (“TBB”) (SEQ ID NO: 24) and (ii) a second-generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H-1Δ”) that lacks each of ITAM2 and ITAM3 and has three (“H-1Δ3”), six (“H-1Δ6”) or nine (“H-1Δ9”) amino acid residues deleted N-terminal to ITAM1. The CCR and the CAR are linked by a furin cleavage site (RRKR (SEQ ID NO: 31)), Ser-Gly linker (SGSG (SEQ ID NO: 32)), and T2A ribosomal skip peptide (EGRGSLLTCGDVEENPGP (SEQ ID NO: 33)).

The protein sequence of the TBB/H-1Δ3 pCAR is shown below as SEQ ID NO: 27. The V_{H} and V_{L} sequences of HMFG2 are underlined and in bold, the ITAM11 sequence of CD3ζ is italicized and in bold.

(SEQ ID NO: 27)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRVKFSRAPAYQQGQNQLYNELNLGRREEYDVLDKRRG.

The protein sequence of the CCR (“TBB”) of TBB/H-1Δ3 is shown above as SEQ ID NO: 24 with the T1E peptide underlined and highlighted in bold.

The protein sequence of the CAR (“H-1Δ3”) of TBB/H-1Δ3 is shown below as SEQ ID NO: 62 with the V_{H} and the V_{L} sequences of HMFG2 underlined and in bold and ITAM1 of CD3ζ italicized and in bold.

(SEQ ID NO: 62)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKFSRAPAYQQGQNQLYNE
LNLGRREEYDVLDKRRG.

The protein sequence of H-1Δ3 of TBB/H-1Δ3 comprises a truncated CD3 polypeptide that has the amino acid sequence of SEQ ID NO: 50 as shown below. The ITAM1 sequence is italicized and in bold.

(SEQ ID NO: 50)
RVKFSRAPAYQQGQNQLYNELNLGRREEYDVLDKRRG

The protein sequence of the TBB/H-1Δ6 pCAR is shown below as SEQ ID NO: 28. The V_{H} and V_{L} sequences of HMFG2 are underlined and in bold, the ITAM1 sequence of CD3ζ is italicized and in bold.

(SEQ ID NO: 28)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRVKAPAYQQGQNQLYNELNLGRREEYDVLDKRRG.

The protein sequence of the CCR (“TBB”) of TBB/H-1Δ6 is shown above as SEQ ID NO: 24 with the T1E peptide underlined and highlighted in bold.

The protein sequence of the CAR (“H-1Δ6”) of TBB/H-1Δ6 is shown below as SEQ ID NO: 63 with the V_{H} and the V_{L} sequences of HMFG2 underlined and in bold and ITAM1 of CD3ζ italicized and in bold.

(SEQ ID NO: 63)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRVKAPAYQQGQNQLYNELNL
GRREEYDVLDKRRG.

The protein sequence of H-1Δ6 of TBB/H-1Δ6 comprises a truncated CD3 polypeptide that has the amino acid sequence of SEQ ID NO: 51 as shown below. The ITAM1 sequence is italicized and in bold.

(SEQ ID NO: 51)
RVKAPAYQQGQNQLYNELNLGRREEYDVLDKRRG.

The protein sequence of TBB/H-1Δ9 pCAR is shown below as SEQ ID NO: 29. The V_{H} and V_{L} sequences of HMFG2 are underlined and in bold, the sequence of the CD28 co-stimulatory region is double underlined, and the ITAM1 sequence of CD3ζ is italicized and in bold.

(SEQ ID NO: 29)
MGPGVLLLLLVATAWHGQGGVVSHFNDCPLSHDGYCLHDGVCMYIE
ALDKYACNCVVGYIGERCQYRDLKWWELRAAAPTTTPAPRPPTPAP
TIASQPLSLRPEACRPAAGGAVHTRGLDFACDIYIWAPLAGTCGVL
LLSLVITLYCNHKRGRKKLLYIFKQPFMRPVQTTQEEDGCSCRFPE
EEEGGCELRRKRSGSGEGRGSLLTCGDVEENPGPMALPVTALLLPL
ALLLHAEVQLQQSGGGLVQPGGSMKLSCVASGFTFSNYWMNWVRQS
PEKGLEWVAEIRLKSNNYATHYAESVKGRFTISRDDSKSSVYLQMN
NLRAEDTGIYYCTFGNSFAYWGQGTTVTVSSGGGGSGGGGSGGGGS
QAVVTQESALTTSPGETVTLTCRSSTGAVTTSNYANWVQEKPDHLF
TGLIGGTNNRAPGVPARFSGSLIGDKAALTITGAQTEDEAIYFCAL
WYSNHWVFGGGTKLTVLGSEAAAIEVMYPPPYLDNEKSNGTIIHVK
GKHLCPSPLFPGPSKPFWVLVVVGGVLACYSLLVTVAFIIFWVRSK
RSRLLHSDYMNMTPRRPGPTRKHYQPYAPPRDFAAYRSAPAYQQGQ
NQLYNELNLGRREEYDVLDKRRG.

The protein sequence of the CCR (“TBB”) of TBB/H-1Δ9 is shown above as SEQ ID NO: 24 with the T1E peptide underlined and highlighted in bold.

The protein sequence of the CAR (“H-1Δ9”) of TBB/H-1Δ9 is shown below as SEQ ID NO: 64 with the V_{H} and the V_{L} sequences of HMFG2 underlined and in bold and ITAM1 of CD3ζ italicized and in bold.

(SEQ ID NO: 64)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSAPAYQQGQNQLYNELNLGRR
EEYDVLDKRRG.

The protein sequence of H-1Δ9 of TBB/H-1Δ9 comprises a truncated CD3 polypeptide that has the amino acid sequence of SEQ ID NO: 52 as shown below. The ITAM1 sequence is italicized and in bold.

(SEQ ID NO: 52)
APAYQQGQNQLYNELNLGRREEYDVLDKRRG.

The TBB/H-1Δ9 YRS pCAR is a modified version of TBB/H-1Δ9 in which the YRS motif of the CD28 co-stimulatory region is deleted.

The protein sequence of TBB/H-1Δ9 YRS is shown below as SEQ ID NO: 30. The V_{H} and V_{L} sequences of HMFG2 are underlined and in bold, the sequence of the CD28 co-stimulatory region is double underlined, and the ITAM1 sequence of CD3ζ is italicized and in bold.

(SEQ ID NO: 30)
MGPGVLLLLLVATAWHGQGGVVSHFNDCPLSHDGYCLHDGVCM
YIEALDKYACNCVVGYIGERCQYRDLKWWELRAAAPTTTPAPR
PPTPAPTIASQPLSLRPEACRPAAGGAVHTRGLDFACDIYIWA
PLAGTCGVLLLSLVITLYCNHKRGRKKLLYIFKQPFMRPVQTT
QEEDGCSCRFPEEEEGGCELRRKRSGSGEGRGSLLTCGDVEEN
PGPMALPVTALLLPLALLLHAEVQLQQSGGGLVQPGGSMKLSC
VASGFTFSNYWMNWVRQSPEKGLEWVAEIRLKSNNYATHYAES
VKGRFTISRDDSKSSVYLQMNNLRAEDTGIYYCTFGNSFAYWG
QGTTVTVSSGGGGSGGGGSGGGGSQAVVTQESALTTSPGETVT
LTCRSSTGAVTTSNYANWVQEKPDHLFTGLIGGTNNRAPGVPA
RFSGSLIGDKAALTITGAQTEDEAIYFCALWYSNHWVFGGGTK
LTVLGSEAAAIEVMYPPPYLDNEKSNGTIIHVKGKHLCPSPLF
PGPSKPFWVLVVVGGVLACYSLLVTVAFIIFWVRSKRSRLLHS
DYMNMTPRRPGPTRKHYQPYAPPRDFAAAPAYQQGQNQLYNEL
NLGRREEYDVLDKRRG.

The protein sequence of the CCR (“TBB”) of TBB/H-1Δ9 YRS is shown above as SEQ ID NO: 24 with the T1E peptide underlined and highlighted in bold.

The protein sequence of the CAR (“H-1Δ9 YRS”) of TBB/H-1Δ9 YRS is shown below as SEQ ID NO: 65 with the V_{H} and the V_{L} sequences of HMFG2 underlined and in bold, the sequence of the CD28 co-stimulatory region double underlined, and the ITAM1 sequence of CD3 italicized and in bold.

(SEQ ID NO: 65)
MALPVTALLLPLALLLHAEVQLQQSGGGLVQPGGSMKLSCVASGFT
FSNYWMNWVRQSPEKGLEWVAEIRLKSNNYATHYAESVKGRFTISR
DDSKSSVYLQMNNLRAEDTGIYYCTFGNSFAYWGQGTTVTVSSGGG
GSGGGGSGGGGSQAVVTQESALTTSPGETVTLTCRSSTGAVTTSNY
ANWVQEKPDHLFTGLIGGTNNRAPGVPARFSGSLIGDKAALTITGA
QTEDEAIYFCALWYSNHWVFGGGTKLTVLGSEAAAIEVMYPPPYLD
NEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFA
AAPAYQQGQNQLYNELNLGRREEYDVLDKRRG.

The protein sequence of H-1Δ9 YRS of TBB/H-1Δ9 YRS comprises a truncated CD3ζ polypeptide that has the amino acid sequence of SEQ ID NO: 53 as shown below. The ITAM1 sequence is italicized and in bold.

(SEQ ID NO: 53)
APAYQQGQNQLYNELNLGRREEYDVLDKRRG.

The protein sequence of the TBB/H-FcεR1γ pCAR is shown below as SEQ ID NO: 54. The V_{H} and V_{L} sequences of HMFG2 are underlined and in bold, the ITAM of FcεR1γ is italicized and in bold.

(SEQ ID NO: 54)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPEGTVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSRLKIQVRKAAITSYEKSDGVYTGLSTRNQETYETLKHEKPPQ.

The protein sequence of the CCR (“TBB”) of TBB/H-FcεR1γ is shown above as SEQ ID NO: 24 with the T1E peptide underlined and highlighted in bold.

The protein sequence of the CAR (“FcεR1γ”) of TBB/H-FcεR1γ is shown below as SEQ ID NO: 55 with the V_{H} and the V_{L} sequences of HMFG2 underlined and in bold, the ITAM of FcεR1γ is italicized and in bold.

(SEQ ID NO: 55)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSRLKIQVRKAAITSYEKSDGV
YTGLSTRNQETYETLKHEKPPQ.

The protein sequence of the TBB/H-DAP12 pCAR is shown below as SEQ ID NO: 56. The V_{H} and V_{L} sequences of HMFG2 are underlined and in bold, the ITAM of DAP12 is italicized and in bold.

(SEQ ID NO: 56)
MGPGVLLLLLVATAWHGQGGVVSHFNDCPLSHDGYCLHDGVCMYIEALD
KYACNCVVGYIGERCQYRDLKWWELRAAAPTTTPAPRPPTPAPTIASQP
LSLRPEACRPAAGGAVHTRGLDFACDIYIWAPLAGTCGVLLLSLVITLY
CNHKRGRKKLLYIFKQPFMRPVQTTQEEDGCSCRFPEEEEGGCELRRKR
SGSGEGRGSLLTCGDVEENPGPMALPVTALLLPLALLLHAEVQLQQSGG
GLVQPGGSMKLSCVASGFTFSNYWMNWVRQSPEKGLEWVAEIRLKSNNY
ATHYAESVKGRFTISRDDSKSSVYLQMNNLRAEDTGIYYCTFGNSFAYW
GQGTTVTVSSGGGGSGGGGSGGGGSQAVVTQESALTTSPGETVTLTCRS
STGAVTTSNYANWVQEKPDHLFTGLIGGTNNRAPGVPARFSGSLIGDKA
ALTITGAQTEDEAIYFCALWYSNHWVFGGGTKLTVLGSEAAAIEVMYPP
PYLDNEKSNGTIIHVKGKHLCPSPLFPGPSKPFWVLVVVGGVLACYSLL
VTVAFIIFWVRSKRSRLLHSDYMNMTPRRPGPTRKHYQPYAPPRDFAAY
RSYFLGRLVPRGRGAAEAATRKQRITETESPYQELQGQRSDVYSDLNTQ
RPYYK.

The protein sequence of the CCR (“TBB”) of TBB/H-DAP12 is shown above as SEQ ID NO: 24 with the T1E peptide underlined and highlighted in bold.

The protein sequence of the CAR (“H-DAP12”) of TBB/H-DAP12 is shown below as SEQ ID NO: 57 with the V_{H} and the V_{L} sequences of HMFG2 underlined and in bold, the ITAM of DAP12 is italicized and in bold.

(SEQ ID NO: 57)
MALPVTALLLPLALLLHAEVQLQQSGGGLVQPGGSMKLSCVASGFTFSN
YWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGG
GGSQAVVTQESALTTSPGETVTLTCRSSTGAVTTSNYANWVQEKPDHLF
TGLIGGTNNRAPGVPARFSGSLIGDKAALTITGAQTEDEAIYFCALWYS
NHWVFGGGTKLTVLGSEAAAIEVMYPPPYLDNEKSNGTIIHVKGKHLCP
SPLFPGPSKPFWVLVVVGGVLACYSLLVTVAFIIFWVRSKRSRLLHSDY
MNMTPRRPGPTRKHYQPYAPPRDFAAYRSYFLGRLVPRGRGAAEAATRK
QRITETESPYQELQGQRSDVYSDLNTQRPYYK.

The NKG2D_bb/H 1XX pCAR (SEQ ID NO: 76) comprises (i) two CCR polypeptides, each comprising a CD124 leader peptide, NKG2D extracellular domain (amino acids 82-216) fused to G4S-G4D linker, IgG1 spacer, followed by a CD8a transmembrane domain and a 4-1BB co-stimulatory domain (“NKG2D_bb”) (SEQ ID NO: 77) and (ii) a second generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H2 1XX”) (SEQ ID NO: 59). The modified CD3ζ polypeptide (SEQ ID NO: 48) comprises a wild-type immunoreceptor tyrosine-based activation motif 1 (ITAM1) and two tyrosine to phenylalanine mutations in each of ITAM2 and ITAM3. The CCR and the CAR are linked by a T2A ribosomal skip peptide.

The protein sequence of NKG2D_bb/H 1XX pCAR is shown below as SEQ ID NO: 76. The NKG2D sequence is single underlined and the ITAMs of the CD3ζ polypeptide are italicized and in bold with the mutated residues double underlined.

(SEQ ID NO: 76)
MGWLCSGLLFPVSCLVLLQVASSGNLFNQEVQIPLTESYCGPCPKNWIC
YKNNCYQFFDESKNWYESQASCMSQNASLLKVYSKEDQDLLKLVKSYHW
MGLVHIPINGSWQWEDGSILSPNLLTIIEMQKGDCALYASSFKGYIENC
STPNTYICMQRTVGGGGSGGGGDEPKSCDKTHTCPPCPIYIWAPLAGTC
GVLLLSLVITLYCNHKRGRKKLLYIFKQPFMRPVQTTQEEDGCSCRFPE
EEEGGCELRRKRSGSGEGRGSLLTCGDVEENPGPMALPVTALLLPLALL
LHAEVQLQQSGGGLVQPGGSMKLSCVASGFTFSNYWMNWVRQSPEKGLE
WVAEIRLKSNNYATHYAESVKGRFTISRDDSKSSVYLQMNNLRAEDTGI
YYCTFGNSFAYWGQGTTVTVSSGGGGSGGGGSGGGGSQAVVTQESALTT
SPGETVTLTCRSSTGAVTTSNYANWVQEKPDHLFTGLIGGTNNRAPGVP
ARFSGSLIGDKAALTITGAQTEDEAIYFCALWYSNHWVFGGGTKLTVLG
SEAAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVL
DKRRGRDPEMGGKPRRKNPQEGLFNELQKDKMAEAFSEIGMKGERRRGK
GHDGLFQGLSTATKDTFDALHMQALPPR.

The protein sequence of the CCR (“NKG2D_bb”) of NKG2D_bb/H 1XX is shown below as SEQ ID NO: 77 with the NKG2D polypeptide sequence in bold and the 4-1BB co-stimulatory signalling region sequence underlined.

(SEQ ID NO: 77)
MGWLCSGLLFPVSCLVLLQVASSGNLFNQEVQIPLTESYCGPCPKNWIC
YKNNCYQFFDESKNWYESQASCMSQNASLLKVYSKEDQDLLKLVKSYHW
MGLVHIPTNGSWQWEDGSILSPNLLTIIEMQKGDCALYASSFKGYIENC
STPNTYICMQRTVGGGGSGGGGDEPKSCDKTHTCPPCPIYIWAPLAGTC
GVLLLSLVITLYCNHKRGRKKLLYIFKQPFMRPVQTTQEEDGCSCRFPE
EEEGGCEL.

The protein sequence of the CAR (“H 1XX”) of NKG2D_bb/H 1XX is shown above as SEQ ID NO: 59.

The NKG2Dbb(trimer)/H 1XX pCAR (SEQ ID NO: 78) comprises (i) three CCR polypeptides, each comprising a CD124 leader peptide, NKG2D extracellular domain (amino acids 82-216) fused to a G4S-G4D-G4 linker, coiled coil domain of Coronin 1A, G4 linker, followed by a 4-1BB transmembrane domain and a 4-1BB co stimulatory domain (“NKG2Dbb(trimer)”) (SEQ ID NO: 79) and (ii) a second generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H2 1XX”) (SEQ ID NO: 59). The modified CD3ζ polypeptide (SEQ ID NO: 48) comprises a wild-type immunoreceptor tyrosine-based activation motif 1 (ITAM1) and two tyrosine to phenylalanine mutations in each of ITAM2 and ITAM3. The CCR and the CAR are linked by a furin cleavage site (RRKR)-SGSG linker-T2A ribosomal skip peptide.

The protein sequence of NKG2Dbb(trimer)/H 1XX pCAR is shown below as SEQ ID NO: 78. The NKG2D sequence is single underlined and the ITAMs of the CD3ζ polypeptide are italicized and in bold with the mutated residues double underlined.

(SEQ ID NO: 78)
MGWLCSGLLFPVSCLVLLQVASSGNLFNQEVQIPLTESYCGPCPKNWICYKNNCYQF
FDESKNWYESQASCMSQNASLLKVYSKEDQDLLKLVKSYHWMGLVHIPTNGSWQWED
GSILSPNLLTIIEMQKGDCALYASSFKGYIENCSTPNTYICMQRTVGGGGSGGGGDG
GGGSGTPSSDAVSRLEEEMRKLQATVQELQKRLDRLEETVQAKGGGGIISFFLALTS
TALLFLLFFLTLRFSVVKRGRKKLLYIFKQPFMRPVQTTQEEDGCSCRFPEEEEGGC
ELRRKRSGSGEGRGSLLTCGDVEENPGPMALPVTALLLPLALLLHAEVQLQQSGGGL
VQPGGSMKLSCVASGFTFSNYWMNWVRQSPEKGLEWVAEIRLKSNNYATHYAESVKG
RFTISRDDSKSSVYLQMNNLRAEDTGIYYCTFGNSFAYWGQGTTVTVSSGGGGSGGG
GSGGGGSQAVVTQESALTTSPGETVTLTCRSSTGAVTTSNYANWVQEKPDHLFTGLI
GGTNNRAPGVPARFSGSLIGDKAALTITGAQTEDEAIYFCALWYSNHWVFGGGTKLT
VLGSEAAAIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPPRDFAAYR
SRVKFSRSADAPAYQQGQNQLYNELNLGRREEYDVLDKRRGRDPEMGGKPRRKNPQE
GL [symbol] NELQKDKMAEA [symbol] SEIGMKGERRRGKGHDGL [symbol] QGLSTATKDT [symbol] DALHMQALPPR.

The protein sequence of the CCR (“NKG2Dbb(trimer)”) of NKG2Dbb(trimer)/H 1XX is shown below as SEQ ID NO: 79 with the NKG2D polypeptide sequence in bold and the 4-1BB co-stimulatory signalling region sequence underlined.

(SEQ ID NO: 79)
MGWLCSGLLFPVSCLVLLQVASSGNLFNQEVQIPLTESYCGPCPKNWICYKNNCYQF
FDESKNWYESQASCMSQNASLLKVYSKEDQDLLKLVKSYHWMGLVHIPTNGSWQWED
GSILSPNLLTIIEMQKGDCALYASSFKGYIENCSTPNTYICMQRTVGGGGSGGGGDG
GGGSGTPSSDAVSRLEEEMRKLQATVQELQKRLDRLEETVQAKGGGGIISFFLALTS
TALLFLLFFLTLRFSVVKRGRKKLLYIFKQPFMRPVQTTQEEDGCSCRFPEEEEGGC
EL.

The protein sequence of the CAR (“H 1XX”) of NKG2D_bb/H 1XX is shown above as SEQ ID NO: 59.

The Pbb(trimer)/H 1XX pCAR (SEQ ID NO: 80) comprises (i) three CCR polypeptides, each comprising a PD-1 polypeptide fused to a G4 linker, coiled coil domain of Coronin 1A, G4 linker, followed by a 4-1BB transmembrane domain and a 4-1BB co-stimulatory domain (“Pbb(trimer)”) (SEQ ID NO: 81) and (ii) a second generation CAR comprising a human MUC1-targeting HMFG2 scFv fused to a portion of the CD28 ectodomain, followed by a CD28 transmembrane domain, a CD28 co-stimulatory domain, and a modified CD3ζ signalling region (“H2 1XX”) (SEQ ID NO: 59). The modified CD3ζ polypeptide (SEQ ID NO: 48) comprises a wild-type immunoreceptor tyrosine-based activation motif 1 (ITAM1) and two tyrosine to phenylalanine mutations in each of ITAM2 and ITAM3. The CCR and the CAR are linked by a furin cleavage site (RRKR)-SGSG linker-T2A ribosomal skip peptide.

The protein sequence of Pbb(trimer)/H 1XX pCAR is shown below as SEQ ID NO: 80. The PD-1 sequence is single underlined and the ITAMs of the CD3ζ polypeptide are italicized and in bold with the mutated residues double underlined.

(SEQ ID NO: 80)
MQIPQAPWPVVWAVLQLGWRPGWELDSPDRPWNPPTESPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHGGGGSGTPSSDAV
SRLEEEMRKLQATVQELQKRLDRLEETVQAKGGGGIISFFLALTSTALLFLLFFLT
LRFSVVKRGRKKLLYIFKQPFMRPVQTTQEEDGCSCRFPEEEEGGCELRRKRSGSG
EGRGSLLTCGDVEENPGPMALPVTALLLPLALLLHAEVQLQQSGGGLVQPGGSMKL
SCVASGFTFSNYWMNWVRQSPEKGLEWVAEIRLKSNNYATHYAESVKGRFTISRDD
SKSSVYLQMNNLRAEDTGIYYCTFGNSFAYWGQGTTVTVSSGGGGSGGGGSGGGGS
QAVVTQESALTTSPGETVTLTCRSSTGAVTTSNYANWVQEKPDHLFTGLIGGTNNR
APGVPARFSGSLIGDKAALTITGAQTEDEAIYFCALWYSNHWVFGGGTKLTVLGSE
AAAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKNPQEGL [symbol]
NELQKDKMAEA [symbol] SEIGMKGERRRGKGHDGL [symbol] QGLSTATKDT [symbol] DALHMQALPPR.

The protein sequence of the CCR (“Pbb(trimer)”) of Pbb(trimer)/H 1XX is shown below as SEQ ID NO: 81 with the PD-1 polypeptide sequence in bold and the 4-1BB co-stimulatory signalling region sequence underlined.

(SEQ ID NO: 81)
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHGGGGSGTPSSDAV
SRLEEEMRKLQATVQELQKRLDRLEETVQAKGGGGIISFFLALTSTALLFLLFFLT
LRFSVVKRGRKKLLYIFKQPFMRPVQTTQEEDGCSCRFPEEEEGGCEL.

The protein sequence of the CAR (“H 1XX”) of NKG2D_bb/H 1XX is shown above as SEQ ID NO: 59.

The 44bb(trimer)/H 1XX pCAR (SEQ ID NO: 82) comprises (i) three CCR polypeptides, each comprising an NKp44 polypeptide fused to a G4 linker, coiled coil domain of Coronin 1A, G4 linker, followed by a 4-1BB transmembrane domain and a 4-1BB co-stimulatory domain (“44bb(trimer)”) (SEQ ID NO: 83) and (ii) a second generation CAR comprising a human MUC1-targeting HMFG2 scFv fused to a portion of the CD28 ectodomain, followed by a CD28 transmembrane domain, a CD28 co-stimulatory domain, and a modified CD3ζ signalling domain (“H2 1XX”) (SEQ ID NO: 59). The modified CD3ζ polypeptide (SEQ ID NO: 48) comprises a wild-type immunoreceptor tyrosine-based activation motif 1 (ITAM1) and two tyrosine to phenylalanine mutations in each of ITAM2 and ITAM3. The CCR and the CAR are linked by a furin cleavage site (RRKR)-SGSG linker-T2A ribosomal skip peptide.

The protein sequence of the 44bb(trimer) pCAR is shown below as SEQ ID NO: 82. The NKp44 sequence is single underlined and the ITAMs of the CD3 polypeptide are italicized and in bold with the mutated residues double underlined.

(SEQ ID NO: 82)
MAWRALHPLLLLLLLFPGSQAQSKAQVLQSVAGQTLTVRCQYPPTGSLYEKKGWCKE
ASALVCIRLVTSSKPRIMAWTSRFTIWDDPDAGFFTVTMTDLREEDSGHYWCRIYRP
SDNSVSKSVRFYLVVSPASASTQTSWTPRDLVSSQTQTQSCVPPTAGARQAPESPST
IPVPSQPQNSTLRPGPAAPIAGGGGSGTPSSDAVSRLEEEMRKLQATVQELQKRLDR
LEETVQAKGGGGIISFFLALTSTALLFLLFFLTLRFSVVKRGRKKLLYIFKQPFMRP
VQTTQEEDGCSCRFPEEEEGGCELRRKRSGSGEGRGSLLTCGDVEENPGPMALPVTA
LLLPLALLLHAEVQLQQSGGGLVQPGGSMKLSCVASGFTFSNYWMNWVRQSPEKGLE
WVAEIRLKSNNYATHYAESVKGRFTISRDDSKSSVYLQMNNLRAEDTGIYYCTFGNS
FAYWGQGTTVTVSSGGGGSGGGGSGGGGSQAVVTQESALTTSPGETVTLTCRSSTGA
VTTSNYANWVQEKPDHLFTGLIGGTNNRAPGVPARFSGSLIGDKAALTITGAQTEDE
AIYFCALWYSNHWVFGGGTKLTVLGSEAAAIEVMYPPPYLDNEKSNGTIIHVKGKHL
CPSPLFPGPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLGRREEYDV
LDKRRGRDPEMGGKPRRKNPQEGL [symbol] NELQKDKMAEA [symbol] SEIGMKGERRRGKGHDGL [symbol] Q
GLSTATKDT [symbol] DALHMQALPPR.

The protein sequence of the CCR (“44bb(trimer)”) of 44bb(trimer)/H 1XX is shown below as SEQ ID NO: 83 with the NKp44 polypeptide sequence in bold and the 4-1BB co-stimulatory signalling region sequenced underlined.

(SEQ ID NO: 83)
MAWRALHPLLLLLLLFPGSQAQSKAQVLQSVAGQTLTVRCQYPPTGSLYEKKGWCKE
ASALVCIRLVTSSKPRTMAWTSRFTIWDDPDAGFFTVTMTDLREEDSGHYWCRIYRP
SDNSVSKSVRFYLVVSPASASTQTSWTPRDLVSSQTQTQSCVPPTAGARQAPESPST
IPVPSQPQNSTLRPGPAAPIAGGGGSGTPSSDAVSRLEEEMRKLQATVQELQKRLDR
LEETVQAKGGGGIISFFLALTSTALLFLLFFLTLRFSVVKRGRKKLLYIFKQPFMRP
VQTTQEEDGCCRFPEEEEGGCEL.

The protein sequence of the CAR (“H 1XX”) of 44bb(trimery/H 1XX is shown above as SEQ ID NO: 59.

5.4.2.1 Other Exemplary pCARs

In some embodiments, the pCAR is I62BB/H 1XX. The CCR in the I62BB/H 1XX pCAR has an ICR62 binding domain fused to a CD8α transmembrane domain and a 4-1BB co-stimulatory domain. The binding element of the CCR is ICR62, an EGFR targeting scFv domain. See Modjtahedi et al., Cell Biophys. 22(1-3):129-146 (1993), incorporated herein by reference in its entirety. The binding element of the CAR is a human MUC1-targeting HMFG2 scFv.

The protein sequence of the I62BB/H 1XX pCAR is shown below as SEQ ID NO: 45. The V_{H} and the V_{L} sequences of HMFG2 and ICR62 are underlined and in bold and the sequence of the CD3ζ signalling region is italicized with the ITAM sequences in bold and italics.

(SEQ ID NO: 45)
MGPGVLLLLLVATAWHGQGGQVNLLQSGAALVKPGASVKLSCKGSGFTFTDYKIHWV
KQSHGKSLEWIGYFNPNSGYSTYNEKFKSKATLTADKSTDTAYMELTSLTSEDSATY
YCTRLSPGGYYVMDAWGQGASVTVSSAQTTAPSVYPLAPGSGGGGSGGGGSGGGGSD
IQMTQSPSFLSASVGDRVTINCKASQNINNYLNWYQQKLGEAPKRLIYNTNNLQTGI
PSRFSGSGSGTDYTLTISSLQPEDFATYFCLQHNSFPTFGAGTKLELKRADAAPTVS
IFPPSKSAAAPTTTPAPRPPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPE
EEEGGCELRRKRSGSGEGRGSLLTCGDVEENPGPMALPVTALLLPLALLLHAEVQLQ
QSGGGLVQPGGSMKLSCVASGFTFSNYWMNWVRQSPEKGLEWVAEIRLKSNNYATHY
AESVKGRFTISRDDSKSSVYLQMNNLRAEDTGIYYCTFGNSFAYWGQGTTVTVSSGG
GGSGGGGSGGGGSQAVVTQESALTTSPGETVTLTCRSSTGAVTTSNYANWVQEKPDH
LFTGLIGGTNNRAPGVPARFSGSLIGDKAALTITGAQTEDEAIYFCALWYSNHWVFG
GGTKLTVLGSEAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDTFDALHMQ
ALPPR.

The protein sequence of the CCR (“I62BB”) of I62BB/H is shown below as SEQ ID NO: 46 with the V_{H} and the V_{L} sequences of ICR62 underlined and highlighted in bold:

(SEQ ID NO: 46)
MGPGVLLLLLVATAWHGQGGQVNLLQSGAALVKPGASVKLSCKGSGFTFTDYKIHWV
KQSHGKSLEWIGYFNPNSGYSTYNEKFKSKATLTADKSTDTAYMELTSLTSEDSATY
YCTRLSPGGYYVMDAWGQGASVTVSSAQTTAPSVYPLAPGSGGGGSGGGGSGGGGSD
IQMTQSPSFLSASVGDRVTINCKASQNINNYLNWYQQKLGEAPKRLIYNTNNLQTGI
PSRFSGSGSGTDYTLTISSLQPEDFATYFCLQHNSFPTFGAGTKLELKRADAAPTVS
IFPPSKSAAAPTITPAPRPPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPE
EEEGGCEL.

The protein sequence of the CAR (“H 1XX”) of I62BB/H 1XX is shown above as SEQ ID NO: 59 with the V_{H} and V_{L} sequences of HMFG2 underlined and in bold and the ITAMs of CD3ζ italicized and in bold.

### 5.5. Adaptor CARs

In one aspect, adaptor chimeric antigen receptors (adaptor CARs) are provided. The adaptor CAR comprises, from C-terminus to N-terminus (from intracellular to extracellular as expressed within an immuno-responsive cell) (i) at least one adaptor protein comprising an activation signalling domain and a co-stimulatory signalling region; and (ii) at least one third binding element that specifically binds a third epitope on a third antigen. The at least one adaptor protein (i) and the at least one third binding element (ii) are expressed as two or more separate polypeptides that associate via non-covalent interaction(s) in the plasma membrane driven by complementary charged amino acids in each polypeptide.

In some embodiments, the adaptor CAR comprises (i) an adaptor protein comprising a CD3ζ activation signalling domain and a Dap10 polypeptide; and (ii) an NKG2D ligand binding domain polypeptide. In some embodiments, the CD3ζ activation signalling domain is modified.

In some embodiments, the modified CD3ζ activation signalling domain comprises at least one ITAM variant comprising one or more loss-of-function mutations. In certain embodiments, the modified CD3ζ activation signalling domain comprises an ITAM2 variant comprising one or more loss-of-function mutations and an ITAM3 variant comprising one or more loss-of-function mutations. In certain embodiments, the ITAM2 variant comprises two loss-of-function mutations and the ITAM3 variant comprises two loss-of-function mutations. In certain embodiments, each of the loss-of-function mutations comprises a tyrosine to phenylalanine substitution.

In some embodiments, the adaptor CAR comprises (i) an adaptor protein comprising a Dap10 co-stimulatory polypeptide and a Dap12 activation signalling domain; and (ii) an NKG2D ligand binding domain polypeptide.

As described elsewhere herein, adaptor CARs are named according to the following convention: (ii) targeting moiety (comprising a binding element that specifically interacts with a target antigen)/(i) co-stimulatory signalling region-activation signalling domain wherein use of a forward slash (/) denotes an adaptor (non-covalent) association between the targeting moiety (ii) and the adaptor protein (signalling unit) (i). The dash (-) in the adaptor CAR name indicates a direct fusion between the two components (e.g. between the co-stimulatory signalling region and the activation signalling domain).

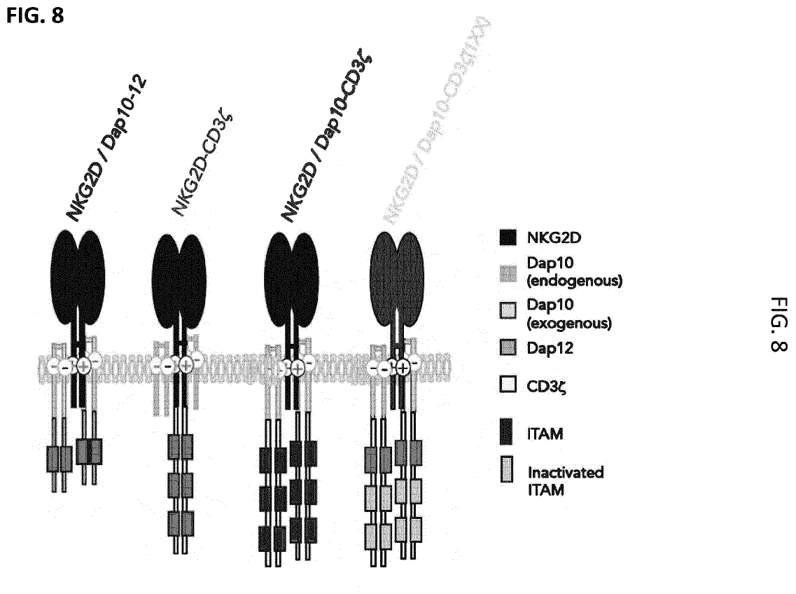

In some embodiments, the adaptor CAR is NKG2D/Dap10-12 as shown in FIG. 8. In some embodiments, the adaptor CAR is NKG2D/Dap10-CD3ζ as shown in FIG. 8. In some embodiments, the adaptor CAR is NKG2D/Dap10-CD3ζ (1XX) as shown in FIG. 8.

### 5.5.1. Adaptor Proteins

The adaptor CAR comprises one or more adaptor proteins. In some embodiments, the adaptor protein comprises an activation signalling domain. In some embodiments, the adaptor protein comprises a co-stimulatory signalling region. In some embodiments, the adaptor protein is a fusion of an activation signalling domain and a co-stimulatory region. In some embodiments, the adaptor protein is a fusion of a modified activation signalling domain and a co-stimulatory region.

In some embodiments, the activation signalling domain of the adaptor protein comprises at least one ITAM. In particular embodiments, the activation signalling domain is Dap12, CD3ζ, or a variant thereof (e.g., CD3ζ 1XX).

In some embodiments, the co-stimulatory signalling region is Dap10 or a variant thereof.

In some embodiments, the adaptor protein is a fusion of an activation signalling domain and a co-stimulatory region that are directly bonded to each other in a contiguous polypeptide chain. In some embodiments, the adaptor protein is a fusion of an activation signalling domain and a co-stimulatory region that are indirectly bonded to each other through a suitable linker (e.g., peptide linker).

Peptide linkers are commonly used in fusion polypeptides and methods for selecting or designing such linkers are well-known. (See, e.g., Chen X et al., 2013, Adv. Drug Deliv. Rev. 65(10): 135701369 and Wriggers W et al., 2005, Biopolymers 80:736-746). Linkers may also be used to join the fusion polypeptide of the disclosure to another polypeptide.

In some embodiments, the adaptor protein is a fusion of a Dap10 polypeptide or functional variant thereof and a CD3ζ polypeptide or a functional variant thereof. In some embodiments, the adaptor protein is a fusion of Dap10 and CD3ζ (e.g., Dap10-CD3ζ in the NKG2D/DAP10-CD3ζ adaptor CAR). In particular embodiments, the adaptor protein is a fusion of Dap10 and CD3ζ 1XX (e.g., Dap10-CD3ζ 1XX in the NKG2D/Dap10-CD3ζ 1XX adaptor CAR).

Adaptor proteins are further described in PCT/EP2020/076566, published as WO 2021/058563, which is incorporated by reference in its entirety.

### 5.5.2. Adaptor CAR Binding Element

In some embodiments, the adaptor CAR binds specifically to an NKG2D ligand (e.g., MICA, MICB, ULBP1-ULBP6).

### 5.5.3. Exemplary Adaptor CARs

The NKG2D/Dap10-12 adaptor CAR (SEQ ID NO: 67) comprises a first polypeptide that is an NKG2D ligand binding domain (SEQ ID NO: 75) and a second polypeptide that is a fusion of a Dap10 co-stimulatory region (SEQ ID NO: 47) and a Dap12 activation signalling domain (SEQ ID NO: 85). The first and second polypeptides are initially expressed as a single mRNA transcript in which the N-terminus of NKG2D is linked to the C-terminus of the DAP12 activation signalling domain by a furin cleavage site (SEQ ID NO: 31), a Ser-Gly linker (SEQ ID NO: 32)), and a P2A ribosomal skip peptide (SEQ ID NO: 34).

The protein sequence of the NKG2D/Dap10-12 adaptor CAR is shown below as SEQ ID NO: 67. Dap10 sequence is bold; Dap12 sequence is underlined with the Dap 12 ITAM in bold; NKG2D sequence is double underlined.

(SEQ ID NO: 67)
MIHLGHILFLLLLPVAAAQTTPGERSSLPAFYPGTSGSCSGCGSLSLPLLAGLVAAD
AVASLLIVGAVFLCARPRRSPAQEDGKVYINMPGRGYFLGRLVPRGRGAAEAATRKQ
RITETESPYQELQGQRSDVYSDLNTQRPYYKRRKRSGSGATNFSLLKQAGDVEENPG
PMGWIRGRRSRHSWEMSEFHNYNLDLKKSDFSTRWQKQRCPVVKSKCRENASPFFFC
CFIAVAMGIRFIIMVAIWSAVFLNSLFNQEVQIPLTESYCGPCPKNWICYKNNCYQF
FDESKNWYESQASCMSQNASLLKVYSKEDQDLLKLVKSYHWMGLVHIPINGSWQWED
GSILSPNLLTIIEMQKGDCALYASSFKGYIENCSTPNTYICMQRTV.

The NKG2D/Dap10-12 adaptor CAR is described in more detail in PCT/EP2020/076566, published as WO 2021/058563, which is incorporated by reference herein in its entirety.

The NKG2D/Dap10-CD3ζ adaptor CAR (SEQ ID NO: 72) comprises a first polypeptide that is an NKG2D ligand binding domain (SEQ ID NO: 75) and a second polypeptide that is a fusion of a Dap10 co-stimulatory polypeptide (SEQ ID NO: 47) and a CD3ζ activation signalling domain (SEQ ID NO: 1). The first and second polypeptides are initially expressed as a single mRNA transcript in which the N-terminus of NKG2D is linked to the C-terminus of CD3ζ by a furin cleavage site (SEQ ID NO: 31), a Ser-Gly linker (SEQ ID NO: 32), and a P2A skip peptide (SEQ ID NO: 34).

The protein sequence of the NKG2D/Dap10-CD3ζ adaptor CAR is shown below as SEQ ID NO: 72. Dap10 sequence is bold; CD3ζ sequence is single underlined with the ITAMs of CD3ζ in italics; NKG2D sequence is double underlined:

(SEQ ID NO: 72)
MIHLGHILFLLLLPVAAAQTTPGERSSLPAFYPGTSGSCSGCGSLSLPLLAGLVAAD
AVASLLIVGAVFLCARPRRSPAQEDGKVYINMPGRGRVKFSRSADAPAYQQGQNQLY
NELNLGRREEYDVLDKRRGRDPEMGGKPRRKNPQEGLYNELQKDKMAEAYSEIGMKG
ERRRGKGHDGLYQGLSTATKDTYDALHMQALPPRRRKRSGSGATNFSLLKQAGDVEE
NPGPMGWIRGRRSRHSWEMSEFHNYNLDLKKSDFSTRWQKQRCPVVKSKCRENASPF
FFCCFIAVAMGIRFIIMVAIWSAVFLNSLFNQEVQIPLTESYCGPCPKNWICYKNNC
YQFFDESKNWYESQASCMSQNASLLKVYSKEDQDLLKLVKSYHWMGLVHIPTNGSWQ
WEDGSILSPNLLTIIEMQKGDCALYASSFKGYIENCSTPNTYICMQRTV.

The NKG2D/Dap10-CD3ζ 1XX adaptor CAR (SEQ ID NO: 74) comprises a first polypeptide that is an NKG2D ligand binding domain (SEQ ID NO: 75) and a second polypeptide that is a fusion of a Dap10 co-stimulatory region and a modified CD3ζ activation signalling domain (SEQ ID NO: 88). The first and second polypeptides are initially expressed as a single mRNA transcript in which the N-terminus of NKG2D is linked to the C-terminus of CD3ζ by a furin cleavage site (SEQ ID NO: 31), a Ser-Gly linker (SEQ ID NO: 32), and P2A skip peptide (SEQ ID NO: 34).

The protein sequence of the NKG2D/Dap10-CD3ζ 1XX adaptor CAR is shown below as SEQ ID NO: 74. Dap10 sequence is bold; CD3ζ sequence is single underlined and ITAMs of CD3ζ are in italics, mutated residues of CD3ζ are double underlined; NKG2D sequence is double underlined.

(SEQ ID NO: 74)
MIHLGHILFLLLLPVAAAQTTPGERSSLPAFYPGTSGSCSGCGSLSLPLLAGLVAAD
AVASLLIVGAVFLCARPRRSPAQEDGKVYINMPGRGRVKFSRSADAPAYQQGQNQLY
NELNLGRREEYDVLDKRRGRDPEMGGKPRRKNPQEGLFNELQKDKMAEAFSEIGMKG
ERRRGKGHDGLFQGLSTATKDTFDALHMQALPPRRRKRSGSGATNFSLLKQAGDVEE
NPGPMGWIRGRRSRHSWEMSEFHNYNLDLKKSDFSTRWQKQRCPVVKSKCRENASPF
FFCCFIAVAMGIRFIIMVAIWSAVFLNSLFNQEVQIPLTESYCGPCPKNWICYKNNC
YQFFDESKNWYESQASCMSQNASLLKVYSKEDQDLLKLVKSYHWMGLVHIPTNGSWQ
WEDGSILSPNLLTIIEMQKGDCALYASSFKGYIENCSTPNTYICMQRTV.

### 5.6. Immunoresponsive Cells

In one aspect, immunoresponsive cells are provided. In some embodiments, the immunoresponsive cells express a second-generation chimeric antigen receptor (CAR) as described herein. In some embodiments, the immunoresponsive cells express a pCAR comprising a second-generation chimeric antigen receptor (CAR) and, in parallel, a chimeric co-stimulatory receptor (CCR).

The second-generation CAR comprises (a) an intracellular signalling domain comprising a modified CD3ζ polypeptide; (b) a co-stimulatory signalling region; (c) a transmembrane domain; and (d) a first binding element that specifically interacts with a first epitope on a first target antigen, wherein the modified CD3ζ polypeptide comprises an immunoreceptor tyrosine-based activation motif 1 (ITAM1), and a mutation in ITAM2 and/or ITAM3.

The pCAR comprises a second-generation CAR and a chimeric co-stimulatory receptor (CCR). The pCAR comprises a second-generation CAR comprising (a) an intracellular signalling domain comprising a modified CD3ζ polypeptide; (b) a co-stimulatory signalling region; (c) a transmembrane domain; and (d) a first binding element that specifically interacts with a first epitope on a first target antigen, wherein the modified CD3ζ polypeptide comprises an immunoreceptor tyrosine-based activation motif 1 (ITAM1), and a mutation in ITAM2 and/or ITAM3, and a CCR comprising (e) a co-stimulatory signalling region which is different from that of (b); (f) a transmembrane domain; and (g) a second binding element that specifically interacts with a second epitope on a second target antigen.

### 5.6.1. Cells

In some embodiments, the immunoresponsive cells are T-cells.

In certain embodiments, the immunoresponsive cells are αβ T-cells. In particular embodiments, the immunoresponsive cells are cytotoxic αβ T-cells. In particular embodiments, the immunoresponsive cells are αβ helper T-cells. In particular embodiments, the immunoresponsive cells are regulatory αβ T-cells (Tregs).

In certain embodiments, the immunoresponsive cells are γδ T-cells. In particular embodiments, the immunoresponsive cells are Vδ2⁺ γδ T-cells. In particular embodiments, the immunoresponsive cells are Vδ2⁻ T-cells. In specific embodiments, the Vδ2⁻ T-cells are Vδ1⁺ cells.

In certain embodiments, the immunoresponsive cells are Natural Killer (NK) cells.

In some embodiments, the immunoresponsive cell expresses no additional exogenous proteins. In other embodiments, the immunoresponsive cell is engineered to express additional exogenous proteins, such as a cytokine, receptor or derivative thereof.

In some embodiments, the immunoresponsive cell is a cell from a cell line. In some embodiments, the immunoresponsive cell is a primary T-cell. In some embodiments, the immunoresponsive cell is a human cell, optionally a human primary T-cell.

In some embodiments, the immunoresponsive cells are obtained from peripheral blood mononuclear cells (PBMCs). In some embodiments, the immuno-responsive cells are obtained from tumours. In some embodiments, the immuno-responsive cells obtained from tumours are tumour-infiltrating lymphocytes (TILs). In some embodiments, the TILs are αβ T cells. In other specific embodiments, the TILs are γδ T-cells, and in particular, Vδ2⁺ or Vδ2⁻ γδ T-cells.

5.7. Nucleic Acids and Methods of Making pCAR T-Cells

Also provided herein is a combination of a first nucleic acid encoding a second-generation CAR as described above and a second nucleic acid encoding a CCR as described above. As indicated above, for convenience herein, the CAR and CCR combination is referred to in the singular as a pCAR, although the CAR and CCR are separate, co-expressed, proteins. Suitable sequences for the nucleic acids will be apparent to a skilled person based on the description of the CAR and CCR above. The sequences may be optimized for use in the required immunoresponsive cell. However, in some cases, as discussed above, codons may be varied from the optimum or “wobbled” in order to avoid repeat sequences. Particular examples of such nucleic acids encode the preferred embodiments described above.

In order to achieve transduction, the nucleic acids encoding the pCAR are suitably introduced into one or more vectors, such as a plasmid or a retroviral or lentiviral vector. Such vectors, including plasmid vectors, or cell lines containing them, form a further aspect of the invention.

In typical embodiments, the immunoresponsive cells are subjected to genetic modification, for example by retroviral or lentiviral mediated transduction, to introduce CAR and CCR coding nucleic acids into the host T-cell genome, thereby permitting stable CAR and CCR expression. They may then be reintroduced into the patient, optionally after expansion, to provide a beneficial therapeutic effect, as described below.

The first and second nucleic acids encoding the CAR and CCR can be expressed from the same vector or different vectors. The present disclosure further provides a kit for the generation of immunoresponsive cells, such as pCAR T-cells described herein. The kit can comprise a combination of a first nucleic acid encoding a second-generation CAR as described above and a second nucleic acid encoding a CCR as described above. In some embodiments, the kit comprises a combination of one or more vectors comprising the first nucleic acid encoding a second-generation CAR and the second nucleic acid encoding a CCR. In some embodiments, the kit further comprises a reagent for use in genetic modification of immunoresponsive cells.

In some embodiments, the method comprises, (i) obtaining T-cells and/or NK cells from a subject, (ii) transducing a polynucleotide(s) or one or more vector(s) encoding the CAR and CCR peptides of the present disclosure into the T-cells and/or NK cells, and (iii) culturing the T-cells and/or NK cells such that the CAR and CCR are expressed.

In some embodiments, the method comprises, (i) obtaining T-cells and/or NK cells from a subject, (ii) transducing a polynucleotide(s) or one or more vector(s) encoding the CAR and the CCR of the present disclosure into the T-cells and/or NK cells, and (iii) culturing the T-cells and/or NK cells such that the CAR, the CCR, and the chimeric cytokine receptor or autocrine loop are expressed.

### 5.8. Pharmaceutical Composition

The present disclosure further provides pharmaceutical compositions comprising the polynucleotide(s) encoding the CAR and CCR, one or more vector(s) encoding the CAR and CCR, or the immunoresponsive cell expressing pCAR described herein. The pharmaceutical compositions can further comprise a pharmaceutically or physiologically acceptable diluent, carrier and/or excipient. The physiologically acceptable diluent, carrier and/or excipient is generally selected to be suitable for the intended mode of administration and can include agents for modifying, maintaining, or preserving, for example, the pH, osmolarity, viscosity, clarity, color, isotonicity, odor, sterility, stability, rate of dissolution or release, adsorption, or penetration of the composition. These carriers can include aqueous or alcoholic/aqueous solutions, emulsions or suspensions, including saline and/or buffered media.

### 5.9. Methods of Using pCAR

In certain embodiments, the polynucleotide(s) encoding the CAR and CCR, one or more vector(s) encoding the CAR and CCR, or the immunoresponsive pCAR cells disclosed herein are useful in therapy to direct a T cell-mediated immune response to a target cell. Thus, in another aspect, methods for directing a T cell-mediated immune response to a target cell in a patient in need thereof are provided. The method comprises administering to the patient a therapeutically effective amount of the one or more polynucleotide constructs, the one or more vectors, or a population of immunoresponsive cells as described above, wherein the binding elements are specific for the target cell. In typical embodiments, the target cell expresses MUC1 and/or one or more NKG2D ligands.

In another aspect, methods for treating cancer in a patient in need thereof are provided. The method comprises administering to the patient a therapeutically effective amount of the one or more polynucleotide constructs, the one or more vectors, or a population of immunoresponsive cells as described above, wherein the binding elements are specific for the target cell. In typical embodiments, the target cell expresses MUC1. In typical embodiments, the target cell expresses one or more NKG2D ligands. In various embodiments, the patient has breast cancer, ovarian cancer, pancreatic cancer, colorectal cancer, lung cancer, gastric cancer, bladder cancer, myeloma, non-Hodgkin lymphoma, prostate cancer, oesophageal cancer, endometrial cancer, hepatobiliary cancer, duodenal carcinoma, thyroid carcinoma, or renal cell carcinoma. In some embodiments, the patient has colon, breast, ovarian, lung, or pancreatic cancer. In some embodiments, the patient has breast cancer. In some embodiments, the patient has tumour cells expressing MUC1. In some embodiments, the patient has been determined to have tumour cells expressing MUC1. In some embodiments, the patient has tumour cells expressing one or more NKG2D ligands. In some embodiments, the patient has been determined to have tumour cells expressing one or more NKG2D ligands.

In some embodiments, the treatment method further comprises the preceding steps of (i) obtaining immunoresponsive cells from a subject, (ii) transducing the immunoresponsive cells with a polynucleotide(s) or one or more vector(s) encoding the CAR and CCR peptides of the present disclosure, and (iii) culturing the immunoresponsive cells such that the CAR and CCR are expressed.

In another aspect, there is provided the immunoresponsive cell, pharmaceutical composition, polynucleotide, set of polynucleotides or kit of the invention for use in therapy. There is also provided the immunoresponsive cell, pharmaceutical composition, polynucleotide, set of polynucleotides or kit of the invention for use in the treatment of cancer. Also provided is the use of the immunoresponsive cell, pharmaceutical composition, polynucleotide, set of polynucleotides or kit of the invention for the manufacture of a medicament for the treatment of cancer. In various embodiments, the patient has breast cancer, ovarian cancer, pancreatic cancer, colorectal cancer, lung cancer, gastric cancer, bladder cancer, myeloma, non-Hodgkin lymphoma, prostate cancer, oesophageal cancer, endometrial cancer, hepatobiliary cancer, duodenal carcinoma, thyroid carcinoma, or renal cell carcinoma. In some embodiments, the patient has colon, breast, ovarian, lung, or pancreatic cancer. In some embodiments, the patient has breast cancer. In some embodiments, the patient has tumour cells expressing MUC1. In some embodiments, the patient has been determined to have tumour cells expressing MUC1. In some embodiments, the patient has tumour cells expressing one or more NKG2D ligands. In some embodiments, the patient has been determined to have tumour cells expressing one or more NKG2D ligands. In some embodiments, the patient has been pre-treated with a chemotherapeutic agent. In some embodiments, the administration of immunoresponsive cells to the patient results in a decrease in tumour size of 10%, 20%, 30%, 40%, 50%, 60%, 70%, 80%, 90%, or even 100%, when compared to an untreated tumour. The amount of immunoresponsive cells administered to the patient should take into account the route of administration, the cancer being treated, the weight of the patient and/or the age of the patient. In general, about 1×10⁶ to about 1×10¹¹ cells are administered to the patient. In one embodiment, about 1×10⁷ to about 1×10¹⁰ cells, or about 1×10⁸ to about 1×10⁹ cells are administered to the patient.

### 6

Sequences

| SEQ ID | | |
|---|---|---|
| NO: | Description | Sequence |
| 1 | CD3ζ (“DA”) | RVKFSRSADAPAYQQGQNQLYNELNLGRREEYDVLDKRRGRDPEMGGKPRRKN |
| | (ITAMs in bold) | PQEGLYNELQKDKMAEAYSEIGMKGERRRGKGHDGLYQGLSTATKDTYDALHM |
| | | QALPPR |
| 2 | CD3ζ variant 1 | RVKFSRSAEPPAYQQGQNQLYNELNLGRREEYDVLDKRRGRDPEMGGKPRRKN |
| | (“EP”) | PQEGLYNELQKDKMAEAYSEIGMKGERRRGKGHDGLYQGLSTATKDTYDALHM |
| | | QALPPR |
| 3 | full length CD28 | MLRLLLALNLFPSIQVTGNKILVKQSPMLVAYDNAVNLSCKYSYNLFSREFRA |
| | protein | SLHKGLDSAVEVCVVYGNYSQQLQVYSKTGFNCDGKLGNESVTFYLQNLYVNQ |
| | (transmembrane | TDIYFCKIEVMYPPPYLDNEKSNGTIIHVKGKHLCPSPLFPGPSKPFWVLVVV |
| | domain in bold) | GGVLACYSLLVTVAFIIFWVRSKRSRLLHSDYMNMTPRRPGPTRKHYQPYAPP |
| | | RDFAAYRS |
| 4 | CD28 co- | IEVMYPPPYLDNEKSNGTIIHVKGKHLCPSPLFPGPSKPFWVLVVVGGVLACY |
| | stimulatory region + | SLLVTVAFIIFWVRSKRSRLLHSDYMNMTPRRPGPTRKHYQPYAPPRDFAAYR |
| | TM domain and | S |
| | partial EC domain | |
| 5 | c-myc tag | EQKLISEEDL |
| 6 | CD28 co- | IEVEQKLISEEDLLDNEKSNGTIIHVKGKHLCPSPLFPGPSKPFWVLVVVGGV |
| | costimulatory | LACYSLLVTVAFIIFWVRSKRSRLLHSDYMNMTPRRPGPTRKHYQPYAPPRDF |
| | region with c-myc | AAYRS |
| | tag | |
| 7 | TBB/H pCAR | MGPGVLLLLLVATAWHGQGGVVSHFNDCPLSHDGYCLHDGVCMYIEALDKYAC |
| | (V_{H} and V_{L} | NCVVGYIGERCQYRDLKWWELRAAAPTTTPAPRPPTPAPTIASQPLSLRPEAC |
| | sequences of | RPAAGGAVHTRGLDFACDIYIWAPLAGTCGVLLLSLVITLYCNHKRGRKKLLY |
| | HMFG2 are | IFKQPFMRPVQTTQEEDGCSCRFPEEEEGGCELRRKRSGSGEGRGSLLTCGDV |
| | underlined and in | EENPGPMALPVTALLLPLALLLHAEVQLQQSGGGLVQPGGSMKLSCVASGFTF |
| | bold, T1E peptide | SNYWMNWVRQSPEKGLEWVAEIRLKSNNYATHYAESVKGRFTISRDDSKSSVY |
| | sequence is | LQMNNLRAEDTGIYYCTFGNSFAYWGQGTTVTVSSGGGGSGGGGSGGGGSQAV |
| | underlined, | VTQESALTTSPGETVTLTCRSSTGAVTTSNYANWVQEKPDHLFTGLIGGTNNR |
| | italicized and in | APGVPARFSGSLIGDKAALTITGAQTEDEAIYFCALWYSNHWVFGGGTKLTVL |
| | bold, and ITAMs | GSEAAAIEVMYPPPYLDNEKSNGTIIHVKGKHLCPSPLFPGPSKPFWVLVVVG |
| | of CD35 are | GVLACYSLLVTVAFIIFWVRSKRSRLLHSDYMNMTPRRPGPTRKHYQPYAPPR |
| | italicized and | DFAAYRSRVKFSRSADAPAYQQGQNQLYNELNLGRREEYDVLDKRRGRDPEMG |
| | bold) | GKPRRKNPQEGLYNELQKDKMAEAYSEIGMKGERRRGKGHDGLYQGLSTATKD |
| | | TYDALHMQALPPR |
| 8 | HMFG2 V_{H} | GFTFSNY |
| | CDR1 | |
| 9 | HMFG2 V_{H} | RLKSNNYA |
| | CDR2 | |
| 10 | HMFG2 V_{H} | GNSFAY |
| | CDR3 | |
| 11 | HMFG2 V_{L} | RSSTGAVTTSNYAN |
| | CDR1 | |
| 12 | HMFG2 V_{L} | GTNNRAP |
| | CDR2 | |
| 13 | HMFG2 V_{L} | ALWYSNHWV |
| | CDR3 | |
| 14 | HMFG2 V_{H} | EVQLQQSGGGLVQPGGSMKLSCVASGFTFSNYWMNWVRQSPEKGLEWVAEIRL |
| | | KSNNYATHYAESVKGRFTISRDDSKSSVYLQMNNLRAEDTGIYYCTFGNSFAY |
| | | WGQGTTVTVSS |
| 15 | HMFG2 V_{L} | QAVVTQESALTTSPGETVTLTCRSSTGAVTTSNYANWVQEKPDHLFTGLIGGT |
| | | NNRAPGVPARFSGSLIGDKAALTITGAQTEDEAIYFCALWYSNHWVFGGGTKL |
| | | TVLGSE |
| 16 | HMFG2 scFv | EVQLQQSGGGLVQPGGSMKLSCVASGFTFSNYWMNWVRQSPEKGLEWVAEIRL |
| | | KSNNYATHYAESVKGRFTISRDDSKSSVYLQMNNLRAEDTGIYYCTFGNSFAY |
| | | WGQGTTVTVSSGGGGSGGGGSGGGGSQAVVTQESALTTSPGETVTLTCRSSTG |
| | | AVTTSNYANWVQEKPDHLFTGLIGGTNNRAPGVPARFSGSLIGDKAALTITGA |
| | | QTEDEAIYFCALWYSNHWVFGGGTKLTVLGSE |
| 17 | HMFG2 scFv | GAGGTGCAGCTGCAGCAGTCTGGAGGAGGCTTGGTGCAACCTGGAGGATCCAT |
| | nucleic acid | GAAACTCTCCTGTGTTGCCTCTGGATTCACTTTCAGTAACTACTGGATGAACT |
| | | GGGTCCGCCAGTCTCCAGAGAAGGGGCTTGAGTGGGTTGCTGAAATTAGATTG |
| | | AAATCTAATAATTATGCAACACATTATGCGGAGTCTGTGAAAGGGAGGTTCAC |
| | | CATCTCAAGAGATGATTCCAAAAGTAGTGTCTACCTGCAAATGAACAACTTAA |
| | | GAGCTGAAGACACTGGCATTTATTACTGTACCTTTGGTAACTCCTTTGCTTAC |
| | | TGGGGCCAAGGGACCACGGTCACCGTCTCCTCAGGTGGAGGCGGTTCAGGCGG |
| | | AGGTGGCTCTGGCGGTGGCGGATCGCAGGCCGTGGTCACTCAGGAATCTGCAC |
| | | TCACCACATCACCTGGTGAAACAGTCACACTCACTTGTCGCTCAAGTACTGGG |
| | | GCTGTTACAACTAGTAACTATGCCAACTGGGTCCAAGAAAAACCAGATCATTT |
| | | ATTCACTGGTCTAATAGGTGGTACCAACAACCGAGCACCAGGTGTTCCTGCCA |
| | | GATTCTCAGGCTCCCTGATTGGAGACAAGGCTGCCCTCACCATCACAGGGGCA |
| | | CAGACTGAGGATGAGGCAATATATTTCTGTGCTCTATGGTACAGCAACCATTG |
| | | GGTGTTCGGTGGAGGAACCAAACTGACTGTCCTAGGATCAGAG |
| 18 | T1E peptide | VVSHFNDCPLSHDGYCLHDGVCMYIEALDKYACNCVVGYIGERCQYRDLKWWE |
| | | LR |
| 19 | T1E nucleic acid | GTGGTGAGCCACTTCAACGACTGCCCTCTGAGCCACGACGGCTACTGCCTGCA |
| | | CGACGGCGTGTGCATGTACATCGAGGCCCTGGACAAGTACGCCTGCAACTGCG |
| | | TGGTGGGCTACATCGGCGAGAGATGCCAGTACAGAGACCTGAAGTGGTGGGAG |
| | | CTGAGA |
| 20 | FcϵR1γ signalling | RLKIQVRKAAITSYEKSDGVYTGLSTRNQETYETLKHEKPPQ |
| | region | |
| 21 | H2 (2G CAR) (V_{H} | MALPVTALLLPLALLLHAEVQLQQSGGGLVQPGGSMKLSCVASGFTFSNYWMN |
| | and V_{L} sequences | WVRQSPEKGLEWVAEIRLKSNNYATHYAESVKGRFTISRDDSKSSVYLQMNNL |
| | of HMFG2 italics | RAEDTGIYYCTFGNSFAYWGQGTTVTVSSGGGGSGGGGSGGGGSQAVVTQESA |
| | and bold; ITAMs | LTTSPGETVTLTCRSSTGAVTTSNYANWVQEKPDHLFTGLIGGTNNRAPGVPA |
| | of CD3ζ are | RFSGSLIGDKAALTITGAQTEDEAIYFCALWYSNHWVFGGGTKLTVLGSEAAA |
| | underlined and | IEVMYPPPYLDNEKSNGTIIHVKGKHLCPSPLFPGPSKPFWVLVVVGGVLACY |
| | bold in sequence) | SLLVTVAFIIFWVRSKRSRLLHSDYMNMTPRRPGPTRKHYQPYAPPRDFAAYR |
| | | SRVKFSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYDALH |
| | | MQALPPR |
| 22 | H-1 CAR | MALPVTALLLPLALLLHAEVQLQQSGGGLVQPGGSMKLSCVASGFTFSNYWMN |
| | (V_{H} and V_{L} | WVRQSPEKGLEWVAEIRLKSNNYATHYAESVKGRFTISRDDSKSSVYLQMNNL |
| | sequences of | RAEDTGIYYCTFGNSFAYWGQGTTVTVSSGGGGSGGGGSGGGGSQAVVTQESA |
| | HMFG2 in italics | LTTSPGETVTLTCRSSTGAVTTSNYANWVQEKPDHLFTGLIGGTNNRAPGVPA |
| | and bold; ITAM1 | RFSGSLIGDKAALTITGAQTEDEAIYFCALWYSNHWVFGGGTKLTVLGSEAAA |
| | of CD3ζ are | IEVMYPPPYLDNEKSNGTIIHVKGKHLCPSPLFPGPSKPFWVLVVVGGVLACY |
| | underlined and in | SLLVTVAFIIFWVRSKRSRLLHSDYMNMTPRRPGPTRKHYQPYAPPRDFAAYR |
| | bold) | SRVKFSRSADAPAYQQGQNQLYNELNLGRREEYDVLDKRRG |
| 23 | TBB/H 1XX (V_{H} | MGPGVLLLLLVATAWHGQGGVVSHFNDCPLSHDGYCLHDGVCMYIEALDKYAC |
| | and V_{L} sequences | NCVVGYIGERCQYRDLKWWELRAAAPTTTPAPRPPTPAPTIASQPLSLRPEAC |
| | of HMFG2 italics | RPAAGGAVHTRGLDFACDIYIWAPLAGTCGVLLLSLVITLYCNHKRGRKKLLY |
| | and bold; ITAMs | IFKQPFMRPVQTTQEEDGCSCRFPEEEEGGCELRRKRSGSGEGRGSLLTCGDV |
| | underlined and | EENPGPMALPVTALLLPLALLLHAEVQLQQSGGGLVQPGGSMKLSCVASGFTF |
| | bold in sequence) | SNYWMNWVRQSPEKGLEWVAEIRLKSNNYATHYAESVKGRFTISRDDSKSSVY |
| | | LQMNNLRAEDTGIYYCTFGNSFAYWGQGTTVTVSSGGGGSGGGGSGGGGSQAV |
| | | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMG |
| | | GKPRRKNPQEGLFNELQKDKMAEAFSEIGMKGERRRGKGHDGLFQGLSTATKD |
| | | TFDALHMQALPPR |
| 24 | TBB CCR (T1E | MGPGVLLLLLVATAWHGQGGVVSHFNDCPLSHDGYCLHDGVCMYIEALDKYAC |
| | underlined and in | NCVVGYIGERCQYRDLKWWELRAAAPTTTPAPRPPTPAPTIASQPLSLRPEAC |
| | bold) | RPAAGGAVHTRGLDFACDIYIWAPLAGTCGVLLLSLVITLYCNHKRGRKKLLY |
| | | IFKQPFMRPVQTTQEEDGCSCRFPEEEEGGCEL |
| 25 | CD28 co- | IEVMYPPPYLDNEKSNGTIIHVKGKHLCPSPLFPGPSKPFWVLVVVGGVLACY |
| | stimulatory region | SLLVTVAFIIFWVRSKRSRLLHSDYMNMTPRRPGPTRKHYQPYAPPRDFAA |
| | with C-term. YRS | |
| | deleted | |
| 26 | TBB/H-1_(V_{H} and | MGPGVLLLLLVATAWHGQGGVVSHFNDCPLSHDGYCLHDGVCMYIEALDKYAC |
| | V_{L} sequences of | NCVVGYIGERCQYRDLKWWELRAAAPTTTPAPRPPTPAPTIASQPLSLRPEAC |
| | HMFG2 italics | RPAAGGAVHTRGLDFACDIYIWAPLAGTCGVLLLSLVITLYCNHKRGRKKLLY |
| | and bold; ITAM1 | IFKQPFMRPVQTTQEEDGCSCRFPEEEEGGCELRRKRSGSGEGRGSLLTCGDV |
| | of CD3ζ | EENPGPMALPVTALLLPLALLLHAEVQLQQSGGGLVQPGGSMKLSCVASGFTF |
| | underlined and | SNYWMNWVRQSPEKGLEWVAEIRLKSNNYATHYAESVKGRFTISRDDSKSSVY |
| | bold) | LQMNNLRAEDTGIYYCTFGNSFAYWGQGTTVTVSSGGGGSGGGGSGGGGSQAV |
| | | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRG |
| 27 | TBB/H-1 Δ3_(V_{H} | MGPGVLLLLLVATAWHGQGGVVSHFNDCPLSHDGYCLHDGVCMYIEALDKYAC |
| | and V_{L} sequences | NCVVGYIGERCQYRDLKWWELRAAAPTTTPAPRPPTPAPTIASQPLSLRPEAC |
| | of HMFG2 italics | RPAAGGAVHTRGLDFACDIYIWAPLAGTCGVLLLSLVITLYCNHKRGRKKLLY |
| | and bold; ITAM1 | IFKQPFMRPVQTTQEEDGCSCRFPEEEEGGCELRRKRSGSGEGRGSLLTCGDV |
| | of CD3ζ is | EENPGPMALPVTALLLPLALLLHAEVQLQQSGGGLVQPGGSMKLSCVASGFTF |
| | underlined and | SNYWMNWVRQSPEKGLEWVAEIRLKSNNYATHYAESVKGRFTISRDDSKSSVY |
| | bold) | LQMNNLRAEDTGIYYCTFGNSFAYWGQGTTVTVSSGGGGSGGGGSGGGGSQAV |
| | | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSRVKFSRAPAYQQGQNQLYNELNLGRREEYDVLDKRRG |
| 28 | TBB/H-1 Δ6_(V_{H} | MGPGVLLLLLVATAWHGQGGVVSHFNDCPLSHDGYCLHDGVCMYIEALDKYAC |
| | and V_{L} sequences | NCVVGYIGERCQYRDLKWWELRAAAPTTTPAPRPPTPAPTIASQPLSLRPEAC |
| | of HMFG2 italics | RPAAGGAVHTRGLDFACDIYIWAPLAGTCGVLLLSLVITLYCNHKRGRKKLLY |
| | and bold; ITAM1 | IFKQPFMRPVQTTQEEDGCSCRFPEEEEGGCELRRKRSGSGEGRGSLLTCGDV |
| | of CD3ζ is | EENPGPMALPVTALLLPLALLLHAEVQLQQSGGGLVQPGGSMKLSCVASGFTF |
| | underlined and | SNYWMNWVRQSPEKGLEWVAEIRLKSNNYATHYAESVKGRFTISRDDSKSSVY |
| | bold) | LQMNNLRAEDTGIYYCTFGNSFAYWGQGTTVTVSSGGGGSGGGGSGGGGSQAV |
| | | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSRVKAPAYQQGQNQLYNELNLGRREEYDVLDKRRG |
| 29 | TBB/H-1 Δ9 | MGPGVLLLLLVATAWHGQGGVVSHFNDCPLSHDGYCLHDGVCMYIEALDKYAC |
| | (V_{H} and V_{L} | NCVVGYIGERCQYRDLKWWELRAAAPTTTPAPRPPTPAPTIASQPLSLRPEAC |
| | sequences of | RPAAGGAVHTRGLDFACDIYIWAPLAGTCGVLLLSLVITLYCNHKRGRKKLLY |
| | HMFG2 italics | IFKQPFMRPVQTTQEEDGCSCRFPEEEEGGCELRRKRSGSGEGRGSLLTCGDV |
| | and bold; ITAM1 | EENPGPMALPVTALLLPLALLLHAEVQLQQSGGGLVQPGGSMKLSCVASGFTF |
| | of CD3ζ is | SNYWMNWVRQSPEKGLEWVAEIRLKSNNYATHYAESVKGRFTISRDDSKSSVY |
| | underlined and | LQMNNLRAEDTGIYYCTFGNSFAYWGQGTTVTVSSGGGGSGGGGSGGGGSQAV |
| | bold) | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSAPAYQQGQNQLYNELNLGRREEYDVLDKRRG |
| 30 | TBB/H-1 Δ9 | MGPGVLLLLLVATAWHGQGGVVSHFNDCPLSHDGYCLHDGVCMYIEALDKYAC |
| | YRS | NCVVGYIGERCQYRDLKWWELRAAAPTTTPAPRPPTPAPTIASQPLSLRPEAC |
| | (V_{H} and V_{L} | RPAAGGAVHTRGLDFACDIYIWAPLAGTCGVLLLSLVITLYCNHKRGRKKLLY |
| | sequences of | IFKQPFMRPVQTTQEEDGCSCRFPEEEEGGCELRRKRSGSGEGRGSLLTCGDV |
| | HMFG2 italics | EENPGPMALPVTALLLPLALLLHAEVQLQQSGGGLVQPGGSMKLSCVASGFTF |
| | and bold; ITAM1 | SNYWMNWVRQSPEKGLEWVAEIRLKSNNYATHYAESVKGRFTISRDDSKSSVY |
| | of CD3ζ | LQMNNLRAEDTGIYYCTFGNSFAYWGQGTTVTVSSGGGGSGGGGSGGGGSQAV |
| | underlined and | VTQESALTTSPGETVTLTCRSSTGAVTTSNYANWVQEKPDHLFTGLIGGTNNR |
| | bold) | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAAPAYQQGQNQLYNELNLGRREEYDVLDKRRG |
| 31 | furin cleavage site | RRKR |
| 32 | Ser-Gly linker | SGSG |
| 33 | T2A peptide | EGRGSLLTCGDVEENPGP |
| 34 | P2A peptide | ATNFSLLKQAGDVEENPGP |
| 35 | CD28 | IEVMYPPPYLDNEKSNGTIIHVKGKHLCPSPLFPGPSKPFWVLVVVGGVLACY |
| | extracellular | SLLVTVAFIIFWV |
| | domain and | |
| | transmembrane | |
| | domain | |
| | (transmembrane | |
| | domain in bold) | |
| 36 | CD8α | PTTTPAPRPPTPAPTIASQPLSLRPEACRPAAGGAVHTRGLDFACDIYIWAPL |
| | extracellular | AGTCGVLLLSLVITLYCNH |
| | domain and | |
| | transmembrane | |
| | domain | |
| 37 | 4-1BB co- | KRGRKKLLYIFKQPFMRPVQTTQEEDGCSCRFPEEEEGGCEL |
| | stimulatory region | |
| 38 | CD27 co- | QRRKYRSNKGESPVEPAEPCHYSCPREEEGSTIPIQEDYRKPEPACSP |
| | stimulatory region | |
| 39 | OX40 co- | ALYLLRRDQRLPPDAHKPPGGGSFRTPIQEEQADAHSTLAKI |
| | stimulatory region | |
| 40 | ICOS co- | CWLTKKKYSSSVHDPNGEYMFMRAVNTAKKSRLTDVTL |
| | stimulatory region | |
| 41 | CD3ζ variant 2 | RVKFSRSADAPAYQQGQNQLYNELNLGRREEYDVLDKRRGRDPEMGGKPQRRK |
| | (UniProt P20963) | NPQEGLYNELQKDKMAEAYSEIGMKGERRRGKGHDGLYQGLSTATKDTYDALH |
| | (ITAMs in bold) | MQALPPR |
| 42 | CD3ζ variant 2 | RVKFSRSADAPAYQQGQNQLYNELNLGRREEYDVLDKRRGRDPEMGGKPQRRK |
| | 1XX with Y to F | NPQEGL [symbol] NELQKDKMAEA [symbol] SEIGMKGERRRGKGHDGL [symbol] QGLSTATKDT [symbol] DALH |
| | mutations in | MQALPPR |
| | ITAM2 and | |
| | ITAM3 (ITAMs | |
| | in bold; mutated | |
| | residues are | |
| | double | |
| | underlined) | |
| 43 | CD3ζ variant 1 | RVKFSRSAEPPAYQQGQNQLYNELNLGRREEYDVLDKRRGRDPEMGGKPRRKN |
| | 1XX with Y to F | PQEGL [symbol] NELQKDKMAEA [symbol] SEIGMKGERRRGKGHDGL [symbol] QGLSTATKDT [symbol] DALHM |
| | mutations in | QALPPR |
| | ITAM2 and | |
| | ITAM3 (ITAMs | |
| | in bold; mutated | |
| | residues are | |
| | double | |
| | underlined) | |
| 44 | ICR62 scFv | QVNLLQSGAALVKPGASVKLSCKGSGFTFTDYKIHWVKQSHGKSLEWIGYENP |
| | | NSGYSTYNEKFKSKATLTADKSTDTAYMELTSLTSEDSATYYCTRLSPGGYYV |
| | | MDAWGQGASVTVSSAQTTAPSVYPLAPGSGGGGSGGGGSGGGGSDIQMTQSPS |
| | | FLSASVGDRVTINCKASQNINNYLNWYQQKLGEAPKRLIYNTNNLQTGIPSRF |
| | | SGSGSGTDYTLTISSLQPEDFATYFCLQHNSFPTFGAGTKLELKRADAAPTVS |
| | | IFPPSKS |
| 45 | I62BB/H 1XX | MGPGVLLLLLVATAWHGQGGQVNLLQSGAALVKPGASVKLSCKGSGFTFTDYK |
| | pCAR (V_{H} and V_{L} | IHWVKQSHGKSLEWIGYFNPNSGYSTYNEKFKSKATLTADKSTDTAYMELTSL |
| | sequences of | TSEDSATYYCTRLSPGGYYVMDAWGQGASVTVSSAQTTAPSVYPLAPGSGGGG |
| | HMFG2 and | SGGGGSGGGGSDIQMTQSPSFLSASVGDRVTINCKASQNINNYLNWYQQKLGE |
| | ICR62 underlined | APKRLIYNTNNLQTGIPSRFSGSGSGTDYTLTISSLQPEDFATYFCLQHNSFP |
| | and in bold; CD3ζ | TFGAGTKLELKRADAAPTVSIFPPSKSAAAPTTTPAPRPPTPAPTIASQPLSL |
| | sequence is | RPEACRPAAGGAVHTRGLDFACDIYIWAPLAGTCGVLLLSLVITLYCKRGRKK |
| | italicized and | LLYIFKQPFMRPVQTTQEEDGCSCRFPEEEEGGCELRRKRSGSGEGRGSLLTC |
| | ITAMs of CD3ζ | GDVEENPGPMALPVTALLLPLALLLHAEVQLQQSGGGLVQPGGSMKLSCVASG |
| | are in bold and | FTFSNYWMNWVRQSPEKGLEWVAEIRLKSNNYATHYAESVKGRFTISRDDSKS |
| | italics; mutated | SVYLQMNNLRAEDTGIYYCTFGNSFAYWGQGTTVTVSSGGGGSGGGGSGGGGS |
| | residues are | QAVVTQESALTTSPGETVTLTCRSSTGAVTTSNYANWVQEKPDHLFTGLIGGT |
| | double | NNRAPGVPARFSGSLIGDKAALTITGAQTEDEAIYFCALWYSNHWVFGGGTKL |
| | underlined) | TVLGSEAAAIEVMYPPPYLDNEKSNGTIIHVKGKHLCPSPLFPGPSKPFWVLV |
| | | VVGGVLACYSLLVTVAFIIFWVRSKRSRLLHSDYMNMTPRRPGPTRKHYQPYA |
| | | PPRDFAAYRSRVKFSRSADAPAYQQGQNQLYNELNLGRREEYDVLDKRRGRDP |
| | | EMGGKPRRKNPQEGL [symbol] NELQKDKMAEA [symbol] SEIGMKGERRRGKGHDGL [symbol] QGLSTA |
| | | TKDT [symbol] DALHMQALPPR |
| 46 | I62BB CCR (V_{H} | MGPGVLLLLLVATAWHGQGGQVNLLQSGAALVKPGASVKLSCKGSGFTFTDYK |
| | and V_{L} sequences | IHWVKQSHGKSLEWIGYFNPNSGYSTYNEKFKSKATLTADKSTDTAYMELTSL |
| | of ICR62 | TSEDSATYYCTRLSPGGYYVMDAWGQGASVTVSSAQTTAPSTYPLAPGSGGGG |
| | underlined and in | SGGGGSGGGGSDIQMTQSPSFLSASVGDRVTINCKASQNINNYLNWYQQKLGE |
| | bold) | APKRLIYNTNNLQTGIPSRFSGSGSGTDYTLTISSLQPEDFATYFCLQHNSFP |
| | | TFGAGTKLELKRADAAPTVSIFPPSKSAAAPTTTPAPRPPTPAPTIASQPLSL |
| | | RPEACRPAAGGAVHTRGLDFACDIYIWAPLAGTCGVLLLSLVITLYCKRGRKK |
| | | LLYIFKQPFMRPVQTTQEEDGCSCRFPEEEEGGCEL |
| 47 | Human Dap10 | MIHLGHILFLLLLPVAAAQTTPGERSSLPAFYPGTSGSCSGCGSLSLPLLAGL |
| | sequence | VAADAVASLLIVGAVELCARPRRSPAQEDGKVYINMPGRG |
| 48 | CD3ζ domain in | RVKFSRSADAPAYQQGQNQLYNELNLGRREEYDVLDKRRGRDPEMGGKPRRKN |
| | H2 1XX, TBB/H | PQEGLFNELQKDKMAEAFSEIGMKGERRRGKGHDGLFQGLSTATKDTFDALHM |
| | 1XX, and | QALPPR |
| | NKG2D/Dap10- | |
| | CD3ζ(1XX) | |
| | (ITAMs in bold; | |
| | mutated residues | |
| | are underlined) | |
| 49 | CD3ζ domain in | RVKFSRSADAPAYQQGQNQLYNELNLGRREEYDVLDKRRG |
| | TBBH-1 (ITAM1 | |
| | underlined and in | |
| | bold) | |
| 50 | CD3ζ domain in | RVKFSRAPAYQQGQNQLYNELNLGRREEYDVLDKRRG |
| | TBBH-1Δ3 | |
| | (ITAM1 | |
| | underlined and in | |
| | bold) | |
| 51 | CD3ζ domain in | RVKAPAYQQGQNQLYNELNLGRREEYDVLDKRRG |
| | TBBH-1Δ6 | |
| | (ITAM1 | |
| | underlined and in | |
| | bold) | |
| 52 | CD3ζ domain in | APAYQQGQNQLYNELNLGRREEYDVLDKRRG |
| | TBBH-1Δ9 | |
| | (ITAM1 | |
| | underlined and in | |
| | bold) | |
| 53 | CD3ζ domain in | APAYQQGQNQLYNELNLGRREEYDVLDKRRG |
| | TBBH-1Δ9 YRS | |
| | (ITAM1 | |
| | underlined and in | |
| | bold) | |
| 54 | TBB/H-FcϵR1γ | MGPGVLLLLLVATAWHGQGGVVSHENDCPLSHDGYCLHDGVCMYIEALDKYAC |
| | (V_{H} and V_{L} | NCVVGYIGERCQYRDLKWWELRAAAPTTTPAPRPPTPAPTIASQPLSLRPEAC |
| | sequences of | RPAAGGAVHTRGLDFACDIYIWAPLAGTCGVLLLSLVITLYCNHKRGRKKLLY |
| | HMFG2 are | IFKQPFMRPVQTTQEEDGCSCRFPEEEEGGCELRRKRSGSGEGRGSLLTCGDV |
| | underlined and in | EENPGPMALPVTALLLPLALLLHAEVQLQQSGGGLVQPGGSMKLSCVASGFTF |
| | bold, the ITAM of | SNYWMNWVRQSPEKGLEWVAEIRLKSNNYATHYAESVKGRFTISRDDSKSSVY |
| | FcϵR1γ_is | LQMNNLRAEDTGIYYCTFGNSFAYWGQGTTVTVSSGGGGSGGGGSGGGGSQAV |
| | underlined and in | VTQESALTTSPGETVTLTCRSSTGAVTTSNYANWVQEKPDHLFTGLIGGTNNR |
| | italics) | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSRLKIQVRKAAITSYEKSDGVYTGLSTRNQETYETLKHEKPPQ |
| 55 | H-FcϵR1γ (MUC1 | MALPVTALLLPLALLLHAEVQLQQSGGGLVQPGGSMKLSCVASGFTFSNYWMN |
| | targeting CAR) | WVRQSPEKGLEWVAEIRLKSNNYATHYAESVKGRFTISRDDSKSSVYLQMNNL |
| | (V_{H} and V_{L} | RAEDTGIYYCTFGNSFAYWGQGTTVTVSSGGGGSGGGGSGGGGSQAVVTQESA |
| | sequences of | LTTSPGETVTLTCRSSTGAVTTSNYANWVQEKPDHLFTGLIGGTNNRAPGVPA |
| | HMFG2 are | RFSGSLIGDKAALTITGAQTEDEAIYFCALWYSNHWVFGGGTKLTVLGSEAAA |
| | underlined and in | IEVMYPPPYLDNEKSNGTIIHVKGKHLCPSPLFPGPSKPFWVLVVVGGVLACY |
| | bold, the ITAM of | SLLVTVAFIIFWVRSKRSRLLHSDYMNMTPRRPGPTRKHYQPYAPPRDFAAYR |
| | FcϵR1γ is in bold | SRLKIQVRKAAITSYEKSDGVYTGLSTRNQETYETLKHEKPPQ |
| | and italics) | |
| 56 | TBB/H-DAP12 | MGPGVLLLLLVATAWHGQGGVVSHFNDCPLSHDGYCLHDGVCMYIEALDKYAC |
| | (V_{H} and V_{L} | NCVVGYIGERCQYRDLKWWELRAAAPTTTPAPRPPTPAPTIASQPLSLRPEAC |
| | sequences of | RPAAGGAVHTRGLDFACDIYIWAPLAGTCGVLLLSLVITLYCNHKRGRKKLLY |
| | HMFG2 are | IFKQPFMRPVQTTQEEDGCSCRFPEEEEGGCELRRKRSGSGEGRGSLLTCGDV |
| | underlined and in | EENPGPMALPVTALLLPLALLLHAEVQLQQSGGGLVQPGGSMKLSCVASGFTF |
| | bold, the ITAM of | SNYWMNWVRQSPEKGLEWVAEIRLKSNNYATHYAESVKGRFTISRDDSKSSVY |
| | DAP12 is in bold | LQMNNLRAEDTGIYYCTFGNSFAYWGQGTTVTVSSGGGGSGGGGSGGGGSQAV |
| | and italics) | VTQESALTTSPGETVTLTCRSSTGAVTTSNYANWVQEKPDHLFTGLIGGTNNR |
| | | APGVPARFSGSLIGDKAALTITGAQTEDEAIYFCALWYSNHWVFGGGTKLTVL |
| | | GSEAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPR |
| | | DFAAYRSYFLGRLVPRGRGAAEAATRKQRITETESPYQELQGQRSDVYSDLNT |
| | | QRPYYK |
| 57 | H-DAP12 (MUC1 | MALPVTALLLPLALLLHAEVQLQQSGGGLVQPGGSMKLSCVASGFTFSNYWMN |
| | targeting CAR) | WVRQSPEKGLEWVAEIRLKSNNYATHYAESVKGRFTISRDDSKSSVYLQMNNL |
| | (V_{H} and V_{L} | RAEDTGIYYCTFGNSFAYWGQGTTVTVSSGGGGSGGGGSGGGGSQAVVTQESA |
| | sequences of | LTTSPGETVTLTCRSSTGAVTTSNYANWVQEKPDHLFTGLIGGTNNRAPGVPA |
| | HMFG2 are | RFSGSLIGDKAALTITGAQTEDEAIYFCALWYSNHWVFGGGTKLTVLGSEAAA |
| | underlined and in | IEVMYPPPYLDNEKSNGTIIHVKGKHLCPSPLFPGPSKPFWVLVVVGGVLACY |
| | bold, the ITAM of | SLLVTVAFIIFWVRSKRSRLLHSDYMNMTPRRPGPTRKHYQPYAPPRDFAAYR |
| | DAP12 is in bold | SYFLGRLVPRGRGAAEAATRKQRITETESPYQELQGQRSDVYSDLNTQRPYYK |
| | and italics) | |
| 58 | DAP12 signalling | YFLGRLVPRGRGAAEAATRKQRITETESPYQELQGQRSDVYSDLNTQRPYYK |
| | region | |
| 59 | H2 1XX | MALPVTALLLPLALLLHAEVQLQQSGGGLVQPGGSMKLSCVASGFTFSNYWMN |
| | (V_{H} and V_{L} | WVRQSPEKGLEWVAEIRLKSNNYATHYAESVKGRFTISRDDSKSSVYLQMNNL |
| | sequences of | RAEDTGIYYCTFGNSFAYWGQGTTVTVSSGGGGSGGGGSGGGGSQAVVTQESA |
| | HMFG2 italics | LTTSPGETVTLTCRSSTGAVTTSNYANWVQEKPDHLFTGLIGGTNNRAPGVPA |
| | and bold; ITAMs | RFSGSLIGDKAALTITGAQTEDEAIYFCALWYSNHWVFGGGTKLTVLGSEAAA |
| | underlined and | IEVMYPPPYLDNEKSNGTIIHVKGKHLCPSPLFPGPSKPFWVLVVVGGVLACY |
| | bold in sequence, | SLLVTVAFIIFWVRSKRSRLLHSDYMNMTPRRPGPTRKHYQPYAPPRDFAAYR |
| | mutated residues | SRVKFSRSADAPAYQQGQNQLYNELNLGRREEYDVLDKRRGRDPEMGGKPRRK |
| | are double | NPQEGL [symbol] NELQKDKMAEA [symbol] SEIGMKGERRRGKGHDGL [symbol] QGLSTATKDT [symbol] DALH |
| | underlined) | MQALPPR |
| 60 | T27 (T1E | MGPGVLLLLLVATAWHGQGGVVSHFNDCPLSHDGYCLHDGVCMYIEALDKYAC |
| | underlined and in | NCVVGYIGERCQYRDLKWWELRAAAPTTTPAPRPPTPAPTIASQPLSLRPEAC |
| | bold) | RPAAGGAVHTRGLDFACDIYIWAPLAGTCGVLLLSLVITLYCNHQRRKYRSNK |
| | | GESPVEPAEPCHYSCPREEEGSTIPIQEDYRKPEPACSP |
| 61 | TOX40 (T1E | MGPGVLLLLLVATAWHGQGGVVSHFNDCPLSHDGYCLHDGVCMYIEALDKYAC |
| | underlined and in | NCVVGYIGERCQYRDLKWWELRAAAPTTTPAPRPPTPAPTIASQPLSLRPEAC |
| | bold) | RPAAGGAVHTRGLDFACDIYIWAPLAGTCGVLLLSLVITLYCNHALYLLRRDQ |
| | | RLPPDAHKPPGGGSFRTPIQEEQADAHSTLAKI |
| 62 | H-1Δ3 (MUC1 | MALPVTALLLPLALLLHAEVQLQQSGGGLVQPGGSMKLSCVASGFTFS |
| | targeting CAR) | NYWMNWVRQSPEKGLEWVAEIRLKSNNYATHYAESVKGRFTISRDDSK |
| | (V_{H} and the V_{L} | SSVYLQMNNLRAEDTGIYYCTFGNSFAYWGQGTTVTVSSGGGGSGGGG |
| | sequences of | SGGGGSQAVVTQESALTTSPGETVTLTCRSSTGAVTTSNYANWVQEKP |
| | HMFG2 | DHLFTGLIGGTNNRAPGVPARFSGSLIGDKAALTITGAQTEDEAIYFC |
| | underlined and in | ALWYSNHWVFGGGTKLTVLGSEAAAIEVMYPPPYLDNEKSNGTIIHVK |
| | bold) | GKHLCPSPLFPGPSKPFWVLVVVGGVLACYSLLVTVAFIIFWVRSKRS |
| | | RLLHSDYMNMTPRRPGPTRKHYQPYAPPRDFAAYRSRVKESRAPAYQQ |
| | | GQNQLYNELNLGRREEYDVLDKRRG |
| 63 | H-1Δ6 (MUC1 | MALPVTALLLPLALLLHAEVQLQQSGGGLVQPGGSMKLSCVASGFTFS |
| | targeting CAR) | NYWMNWVRQSPEKGLEWVAEIRLKSNNYATHYAESVKGRFTISRDDSK |
| | (V_{H} and the V_{L} | SSVYLQMNNLRAEDTGIYYCTFGNSFAYWGQGTTVTVSSGGGGSGGGG |
| | sequences of | SGGGGSQAVVTQESALTTSPGETVTLTCRSSTGAVTTSNYANWVQEKP |
| | HMFG2 | DHLFTGLIGGTNNRAPGVPARFSGSLIGDKAALTITGAQTEDEAIYFC |
| | underlined and in | ALWYSNHWVFGGGTKLTVLGSEAAAIEVMYPPPYLDNEKSNGTIIHVK |
| | bold; ITAM1 of | GKHLCPSPLFPGPSKPFWVLVVVGGVLACYSLLVTVAFIIFWVRSKRS |
| | CD3ζ in italics) | RLLHSDYMNMTPRRPGPTRKHYQPYAPPRDFAAYRSRVKAPAYQQGQN |
| | | QLYNELNLGRREEYDVLDKRRG |
| 64 | H-1Δ9 (MUC1 | MALPVTALLLPLALLLHAEVQLQQSGGGLVQPGGSMKLSCVASGFTFS |
| | targeting CAR) | NYWMNWVRQSPEKGLEWVAEIRLKSNNYATHYAESVKGRFTISRDDSK |
| | (V_{H} and the V_{L} | SSVYLQMNNLRAEDTGIYYCTFGNSFAYWGQGTTVTVSSGGGGSGGGG |
| | sequences of | SGGGGSQAVVTQESALTTSPGETVTLTCRSSTGAVTTSNYANWVQEKP |
| | HMFG2 | DHLFTGLIGGTNNRAPGVPARFSGSLIGDKAALTITGAQTEDEAIYFC |
| | underlined and in | ALWYSNHWVFGGGTKLTVLGSEAAAIEVMYPPPYLDNEKSNGTIIHVK |
| | bold; ITAM1 of | GKHLCPSPLFPGPSKPFWVLVVVGGVLACYSLLVTVAFIIFWVRSKRS |
| | CD3ζ in italics) | RLLHSDYMNMTPRRPGPTRKHYQPYAPPRDFAAYRSAPAYQQGQNQLY |
| | | NELNLGRREEYDVLDKRRG |
| 65 | H-1Δ9 YRS | MALPVTALLLPLALLLHAEVQLQQSGGGLVQPGGSMKLSCVASGFTFSNYWMN |
| | (MUC1 targeting | WVRQSPEKGLEWVAEIRLKSNNYATHYAESVKGRFTISRDDSKSSVYLQMNNL |
| | CAR) | RAEDTGIYYCTFGNSFAYWGQGTTVTVSSGGGGSGGGGSGGGGSQAVVTQESA |
| | (V_{H} and the V_{L} | LTTSPGETVTLTCRSSTGAVTTSNYANWVQEKPDHLFTGLIGGTNNRAPGVPA |
| | sequences of | RFSGSLIGDKAALTITGAQTEDEAIYFCALWYSNHWVFGGGTKLTVLGSEAAA |
| | HMFG2 | IEVMYPPPYLDNEKSNGTIIHVKGKHLCPSPLFPGPSKPFWVLVVVGGVLACY |
| | underlined and in | SLLVTVAFIIFWVRSKRSRLLHSDYMNMTPRRPGPTRKHYQPYAPPRDFAAAP |
| | bold; ITAM1 of | AYQQGQNQLYNELNLGRREEYDVLDKRRG |
| | CD3ζ in italics) | |
| 66 | NKG2D/Dap10- | atgatccacctgggccacatcctgttcctgctgctgctgcccgtggccgctgc |
| | 12 nucleic acid | ccagaccacccctggcgagcggagcagcctgcctgccttctaccctggcacca |
| | | gcggcagctgcagcggctgcggcagcctgagcctgcccctgctggccggcctg |
| | | gtggccgccgacgccgtggccagcctgctgatcgtgggcgccgtgttcctgtg |
| | | cgccaggcccaggcggagccctgcccaggaggacggcaaggtgtacatcaaca |
| | | tgcccggccggggctacttcctgggcaggctggtgcccaggggcaggggcgct |
| | | gccgaggctgccacccggaagcagcggatcaccgagaccgagagcccctacca |
| | | ggagctgcagggccagcggagcgacgtgtacagcgacctgaacacccagaggc |
| | | cctactacaagaggcggaaaaggtctgggagtggggctaccaatttctctctc |
| | | ctcaagcaagccggagacgttgaggaaaaccctggacccatgggctggatccg |
| | | gggacggaggagccggcacagctgggagatgagcgagttccacaactacaacc |
| | | tggacctgaagaagagcgacttcagcacccggtggcagaagcagcggtgcccc |
| | | gtggtgaagagcaagtgccgggagaacgccagccccttcttcttctgctgctt |
| | | catcgccgtggctatgggcatccggttcatcatcatggtggccatctggagcg |
| | | ccgtgttcctgaacagcctgttcaaccaggaggtgcagatccccctgaccgag |
| | | agctactgcggcccctgccccaagaactggatctgctacaagaacaactgcta |
| | | ccagttcttcgacgagagcaagaactggtacgagagccaggccagctgcatga |
| | | gccagaacgccagcctgctgaaggtgtacagcaaggaggaccaggacctgctg |
| | | aagctggtgaagagctaccactggatgggcctggtgcacatccccaccaacgg |
| | | cagctggcagtgggaggacggcagcatcctgagccccaacctgctgaccatca |
| | | tcgagatgcagaagggcgactgcgccctgtacgccagcagcttcaagggctac |
| | | atcgagaactgcagcacccccaacacctacatctgcatgcagcggaccgtgta |
| | | a |
| 67 | NKG2D/Dap10- | MIHLGHILFLLLLPVAAAQTTPGERSSLPAFYPGTSGSCSGCGSLSLPLLAGL |
| | 12 (Dap10 | VAADAVASLLIVGAVFLCARPRRSPAQEDGKVYINMPGRGYFLGRLVPRGRGA |
| | sequence is bold | AEAATRKQRITETESPYQELQGQRSDVYSDLNTQRPYYKRRKRSGSGATNFSL |
| | and not italicized; | LKQAGDVEENPGPMGWIRGRRSRHSWEMSEFHNYNLDLKKSDFSTRWQKQRCP |
| | Dap12 sequence | VVKSKCRENASPFFFCCFIAVAMGIRFIIMVAIWSAVFLNSLFNQEVQIPLTE |
| | is italicized and | SYCGPCPKNWICYKNNCYQFFDESKNWYESQASCMSQNASLLKVYSKEDQDLL |
| | underlined, the | KLVKSYHWMGLVHIPTNGSWQWEDGSILSPNLLTIIEMQKGDCALYASSFKGY |
| | ITAM of DAP12 | IENCSTPNTYICMQRTV |
| | is bold; NKG2D | |
| | sequence is | |
| | double | |
| | underlined) | |
| 68 | NKG2D-CD32 | atgagagtgaagttcagcaggagcgcagacgcccccgcgtaccagcagggcca |
| | nucleic acid | gaaccagctctataacgagctcaatctaggacgaagagaggagtacgatgttt |
| | sequence | tggacaagagacgtggccgggaccctgagatggggggaaagccgagaaggaag |
| | | aaccctcaggaaggcctgtacaatgaactgcagaaagataagatggcggaggc |
| | | ctacagtgagattgggatgaaaggcgagcgccggaggggcaaggggcacgatg |
| | | gcctttaccagggtctcagtacagccaccaaggacacctacgatgcattgcac |
| | | atgcaggccctgccccctcgcggctggatccgcggccgcaggagccggcacag |
| | | ctgggagatgagcgagttccacaactacaacctggacctgaagaagagcgact |
| | | tcagcacccggtggcagaagcagcggtgccccgtggtgaagagcaagtgccgg |
| | | gagaacgccagccccttcttcttctgctgcttcatcgccgtggctatgggcat |
| | | ccggtttataatcatggtggccatctggagcgccgtgttcctgaacagcctgt |
| | | tcaaccaggaggtgcagatccccctgaccgagagctactgcggcccctgcccc |
| | | aagaactggatctgctacaagaacaactgctaccagttcttcgacgagagcaa |
| | | gaactggtacgagagccaggccagctgcatgagccagaacgccagcctgctga |
| | | aggtgtacagcaaggaggaccaggacctgctgaagctggtgaagagctaccac |
| | | tggatgggcctggtgcacatccccaccaacggcagctggcagtgggaggacgg |
| | | cagcatcctgagccccaacctgctgaccatcatcgagatgcagaagggcgact |
| | | gcgccctgtacgccagcagcttcaagggctacatcgagaactgcagcaccccc |
| | | aacacctacatctgcatgcagcggaccgtgtaa |
| 69 | NKG2D-CD3 | MRVKFSRSADAPAYQQGQNQLYNELNLGRREEYDVLDKRRGRDPEMGGKPRRK |
| | CAR (CD35 | NPQEGLYNELQKDKMAEAYSEIGMKGERRRGKGHDGLYQGLSTATKDTYDALH |
| | sequence is | MQALPPRGWIRGRRSRHSWEMSEFHNYNLDLKKSDFSTRWQKQRCPVVKSKCR |
| | italicized and | ENASPFFFCCFIAVAMGIRFIIMVAIWSAVFLNSLFNQEVQIPLTESYCGPCP |
| | ITAMs of CD35 | KNWICYKNNCYQFFDESKNWYESQASCMSQNASLLKVYSKEDQDLLKLVKSYH |
| | are bold; NKG2D | WMGLVHIPTNGSWQWEDGSILSPNLLTIIEMQKGDCALYASSFKGYIENCSTP |
| | sequence is | NTYICMQRTV |
| | double | |
| | underlined) | |
| 70 | [Not assigned] | |
| 71 | NKG2D/Dap10- | atgatccacctgggccacatcctgttcctgctgctgctgcccgtggccgctgc |
| | CD3ζ nucleic | ccagaccacccctggcgagcggagcagcctgcctgccttctaccctggcacca |
| | acid sequence | gcggcagctgcagcggctgcggcagcctgagcctgcccctgctggccggcctg |
| | | gtggccgccgacgccgtggccagcctgctgatcgtgggcgccgtgttcctgtg |
| | | cgccaggcccaggcggagccctgcccaggaggacggcaaggtgtacatcaaca |
| | | tgcccggccggggcagagtgaagttcagcaggagcgcagacgcccccgcgtac |
| | | cagcagggccagaaccagctctataacgagctcaatctaggacgaagagagga |
| | | gtacgatgttttggacaagagacgtggccgggaccctgagatggggggaaagc |
| | | cgagaaggaagaaccctcaggaaggcctgtacaatgaactgcagaaagataag |
| | | atggcggaggcctacagtgagattgggatgaaaggcgagcgccggaggggcaa |
| | | ggggcacgatggcctttaccagggtctcagtacagccaccaaggacacctacg |
| | | atgcattgcacatgcaggccctgccccctcgcaggcggaaaaggtctgggagt |
| | | ggggctaccaatttctctctcctcaagcaagccggagacgttgaggaaaaccc |
| | | tggacccatgggctggatccgcggccgcaggagccggcacagctgggagatga |
| | | gcgagttccacaactacaacctggacctgaagaagagcgacttcagcacccgg |
| | | tggcagaagcagcggtgccccgtggtgaagagcaagtgccgggagaacgccag |
| | | ccccttcttcttctgctgcttcatcgccgtggctatgggcatccggtttataa |
| | | tcatggtggccatctggagcgccgtgttcctgaacagcctgttcaaccaggag |
| | | gtgcagatccccctgaccgagagctactgcggcccctgccccaagaactggat |
| | | ctgctacaagaacaactgctaccagttcttcgacgagagcaagaactggtacg |
| | | agagccaggccagctgcatgagccagaacgccagcctgctgaaggtgtacagc |
| | | aaggaggaccaggacctgctgaagctggtgaagagctaccactggatgggcct |
| | | ggtgcacatccccaccaacggcagctggcagtgggaggacggcagcatcctga |
| | | gccccaacctgctgaccatcatcgagatgcagaagggcgactgcgccctgtac |
| | | gccagcagcttcaagggctacatcgagaactgcagcacccccaacacctacat |
| | | ctgcatgcagcggaccgtgtaa |
| 72 | NKG2D/Dap10- | MIHLGHILFLLLLPVAAAQTTPGERSSLPAFYPGTSGSCSGCGSLSLPLLAGL |
| | CD3ζ (Dap10 | VAADAVASLLIVGAVELCARPRRSPAQEDGKVYINMPGRGRVKFSRSADAPAY |
| | sequence is bold; | QQGQNQLYNELNLGRREEYDVLDKRRGRDPEMGGKPRRKNPQEGLYNELQKDK |
| | CD3ζ sequence is | MAEAYSEIGMKGERRRGKGHDGLYQGLSTATKDTYDALHMQALPPRRRKRSGS |
| | single underlined | GATNFSLLKQAGDVEENPGPMGWIRGRRSRHSWEMSEFHNYNLDLKKSDFSTR |
| | with ITAMs of | WQKQRCPVVKSKCRENASPFFFCCFIAVAMGIRFIIMVAIWSAVFLNSLFNQE |
| | CD3ζ italicized; | VQIPLTESYCGPCPKNWICYKNNCYQFFDESKNWYESQASCMSQNASLLKVYS |
| | NKG2D sequence | KEDQDLLKLVKSYHWMGLVHIPTNGSWQWEDGSILSPNLLTIIEMQKGDCALY |
| | is double | ASSFKGYIENCSTPNTYICMQRTV |
| | underlined) | |
| 73 | NKG2D/Dap10- | atgatccacctgggccacatcctgttcctgctgctgctgcccgtggccgctgc |
| | CD3ζ 1XX | ccagaccacccctggcgagcggagcagcctgcctgccttctaccctggcacca |
| | nucleic acid | gcggcagctgcagcggctgcggcagcctgagcctgcccctgctggccggcctg |
| | sequence | gtggccgccgacgccgtggccagcctgctgatcgtgggcgccgtgttcctgtg |
| | | cgccaggcccaggcggagccctgcccaggaggacggcaaggtgtacatcaaca |
| | | tgcccggccggggcagagtgaagttcagcaggagcgcagacgcccccgcgtac |
| | | cagcagggccagaaccagctctataacgagctcaatctaggacgaagagagga |
| | | gtacgatgttttggacaagagacgtggccgggaccctgagatggggggaaagc |
| | | cgagaaggaagaaccctcaggaaggcctgttcaatgaactgcagaaagataag |
| | | atggcggaggccttcagtgagattgggatgaaaggcgagcgccggaggggcaa |
| | | ggggcacgatggccttttccagggtctcagtacagccaccaaggacaccttcg |
| | | atgcattgcacatgcaggccctgccccctcgcaggcggaaaaggtctgggagt |
| | | ggggctaccaatttctctctcctcaagcaagccggagacgttgaggaaaaccc |
| | | tggacccatgggctggatccgcggccgcaggagccggcacagctgggagatga |
| | | gcgagttccacaactacaacctggacctgaagaagagcgacttcagcacccgg |
| | | tggcagaagcagcggtgccccgtggtgaagagcaagtgccgggagaacgccag |
| | | ccccttcttcttctgctgcttcatcgccgtggctatgggcatccggtttataa |
| | | tcatggtggccatctggagcgccgtgttcctgaacagcctgttcaaccaggag |
| | | gtgcagatccccctgaccgagagctactgcggcccctgccccaagaactggat |
| | | ctgctacaagaacaactgctaccagttcttcgacgagagcaagaactggtacg |
| | | agagccaggccagctgcatgagccagaacgccagcctgctgaaggtgtacagc |
| | | aaggaggaccaggacctgctgaagctggtgaagagctaccactggatgggcct |
| | | ggtgcacatccccaccaacggcagctggcagtgggaggacggcagcatcctga |
| | | gccccaacctgctgaccatcatcgagatgcagaagggcgactgcgccctgtac |
| | | gccagcagcttcaagggctacatcgagaactgcagcacccccaacacctacat |
| | | ctgcatgcagcggaccgtgtaa |
| 74 | NKG2D/Dap10- | MIHLGHILFLLLLPVAAAQTTPGERSSLPAFYPGTSGSCSGCGSLSLPLLAGL |
| | CD3ζ 1XX | VAADAVASLLIVGAVFLCARPRRSPAQEDGKVYINMPGRGRVKFSRSADAPAY |
| | (Dap10 sequence | QQGQNQLYNELNLGRREEYDVLDKRRGRDPEMGGKPRRKNPQEGLFNELQKDK |
| | is bold; CD3ζ | MAEAFSEIGMKGERRRGKGHDGLFQGLSTATKDTFDALHMQALPPRRRKRSGS |
| | sequence is single | GATNFSLLKQAGDVEENPGPMGWIRGRRSRHSWEMSEFHNYNLDLKKSDFSTR |
| | underlined with | WQKQRCPVVKSKCRENASPFFFCCFIAVAMGIRFIIMVAIWSAVFLNSLFNQE |
| | ITAMs of CD35 | VQIPLTESYCGPCPKNWICYKNNCYQFFDESKNWYESQASCMSQNASLLKVYS |
| | italicized and | KEDQDLLKLVKSYHWMGLVHIPTNGSWQWEDGSILSPNLLTIIEMQKGDCALY |
| | mutated residues | ASSFKGYIENCSTPNTYICMQRTV |
| | of CD3ζ double | |
| | underlined; | |
| | NKG2D sequence | |
| | is double | |
| | underlined) | |
| 75 | Human NKG2D | MGWIRGRRSRHSWEMSEFHNYNLDLKKSDFSTRWQKQRCPVVKSKCRENASPF |
| | sequence | FFCCFIAVAMGIRFIIMVAIWSAVFLNSLFNQEVQIPLTESYCGPCPKNWICY |
| | | KNNCYQFFDESKNWYESQASCMSQNASLLKVYSKEDQDLLKLVKSYHWMGLVH |
| | | IPTNGSWQWEDGSILSPNLLTIIEMQKGDCALYASSFKGYIENCSTPNTYICM |
| | | QRTV |
| 76 | NKG2D_bb/H | MGWLCSGLLFPVSCLVLLQVASSGNLFNQEVQIPLTESYCGPCPKNWICYKNN |
| | 1XX pCAR | CYQFFDESKNWYESQASCMSQNASLLKVYSKEDQDLLKLVKSYHWMGLVHIPT |
| | sequence. The | NGSWQWEDGSILSPNLLTIIEMQKGDCALYASSFKGYIENCSTPNTYICMQRT |
| | NKG2D sequence | VGGGGSGGGGDEPKSCDKTHTCPPCPIYIWAPLAGTCGVLLLSLVITLYCNHK |
| | is single | RGRKKLLYIFKQPFMRPVQTTQEEDGCSCRFPEEEEGGCELRRKRSGSGEGRG |
| | underlined and the | SLLTCGDVEENPGPMALPVTALLLPLALLLHAEVQLQQSGGGLVQPGGSMKLS |
| | ITAMs of the | CVASGFTFSNYWMNWVRQSPEKGLEWVAEIRLKSNNYATHYAESVKGRFTISR |
| | CD3ζ polypeptide | DDSKSSVYLQMNNLRAEDTGIYYCTFGNSFAYWGQGTTVTVSSGGGGSGGGGS |
| | are italicized and | GGGGSQAVVTQESALTTSPGETVTLTCRSSTGAVTTSNYANWVQEKPDHLFTG |
| | in bold with the | LIGGTNNRAPGVPARFSGSLIGDKAALTITGAQTEDEAIYFCALWYSNHWVFG |
| | mutated residues | GGTKLTVLGSEAAAIEVMYPPPYLDNEKSNGTIIHVKGKHLCPSPLFPGPSKP |
| | double underlined | FWVLVVVGGVLACYSLLVTVAFIIFWVRSKRSRLLHSDYMNMTPRRPGPTRKH |
| | | YQPYAPPRDFAAYRSRVKFSRSADAPAYQQGQNQLYNELNLGRREEYDVLDKR |
| | | RGRDPEMGGKPRRKNPQEGL [symbol] NELQKDKMAEA [symbol] SEIGMKGERRRGKGHDGL [symbol] Q |
| | | GLSTATKDT [symbol] DALHMQALPPR |
| 77 | NKG2D_bb CCR | MGWLCSGLLFPVSCLVLLQVASSGNLFNQEVQIPLTESYCGPCPKNWICYKNN |
| | sequence. The | CYQFFDESKNWYESQASCMSQNASLLKVYSKEDQDLLKLVKSYHWMGLVHIPT |
| | NKG2D | NGSWQWEDGSILSPNLLTIIEMQKGDCALYASSFKGYIENCSTPNTYICMQRT |
| | polypeptide | VGGGGSGGGGDEPKSCDKTHTCPPCPIYIWAPLAGTCGVLLLSLVITLYCNHK |
| | sequence in bold | RGRKKLLYIFKQPFMRPVQTTQEEDGCSCRFPEEEEGGCEL |
| | and the 4-1BB co- | |
| | stimulatory | |
| | signalling region | |
| | sequence | |
| | underlined. | |
| 78 | NKG2Dbb(trimer)/ | MGWLCSGLLFPVSCLVLLQVASSGNLFNQEVQIPLTESYCGPCPKNWICYKNN |
| | H 1XX pCAR | CYQFFDESKNWYESQASCMSQNASLLKVYSKEDQDLLKLVKSYHWMGLVHIPT |
| | sequence. The | NGSWQWEDGSILSPNLLTIIEMQKGDCALYASSFKGYIENCSTPNTYICMQRT |
| | NKG2D sequence | VGGGGSGGGGDGGGGSGTPSSDAVSRLEEEMRKLQATVQELQKRLDRLEETVQ |
| | is single | AKGGGGIISFFLALTSTALLFLLFFLTLRFSVVKRGRKKLLYIFKQPFMRPVQ |
| | underlined and the | TTQEEDGCSCRFPEEEEGGCELRRKRSGSGEGRGSLLTCGDVEENPGPMALPV |
| | ITAMs of the | TALLLPLALLLHAEVQLQQSGGGLVQPGGSMKLSCVASGFTFSNYWMNWVRQS |
| | CD3ζ polypeptide | PEKGLEWVAEIRLKSNNYATHYAESVKGRFTISRDDSKSSVYLQMNNLRAEDT |
| | are italicized and | GIYYCTFGNSFAYWGQGTTVTVSSGGGGSGGGGSGGGGSQAVVTQESALTTSP |
| | in bold with the | GETVTLTCRSSTGAVTTSNYANWVQEKPDHLFTGLIGGTNNRAPGVPARFSGS |
| | mutated residues | LIGDKAALTITGAQTEDEAIYFCALWYSNHWVFGGGTKLTVLGSEAAAIEVMY |
| | double | PPPYLDNEKSNGTIIHVKGKHLCPSPLFPGPSKPFWVLVVVGGVLACYSLLVT |
| | underlined. | VAFIIFWVRSKRSRLLHSDYMNMTPRRPGPTRKHYQPYAPPRDFAAYRSRVKF |
| | | SRSADAPAYQQGQNQLYNELNLGRREEYDVLDKRRGRDPEMGGKPRRKNPQEG |
| | | L [symbol] NELQKDKMAEA [symbol] SEIGMKGERRRGKGHDGL [symbol] QGLSTATKDT [symbol] DALHMQALP |
| | | PR |
| 79 | NKG2D(trimer) | MGWLCSGLLFPVSCLVLLQVASSGNLFNQEVQIPLTESYCGPCPKNWICYKNN |
| | CCR sequence. | CYQFFDESKNWYESQASCMSQNASLLKVYSKEDQDLLKLVKSYHWMGLVHIPT |
| | The NKG2D | NGSWQWEDGSILSPNLLTIIEMQKGDCALYASSFKGYIENCSTPNTYICMQRT |
| | polypeptide | VGGGGSGGGGDGGGGSGTPSSDAVSRLEEEMRKLQATVQELQKRLDRLEETVQ |
| | sequence in bold | AKGGGGIISFFLALTSTALLFLLFFLTLRFSVVKRGRKKLLYIFKQPFMRPVQ |
| | and the 4-1BB co- | TTQEEDGCSCRFPEEEEGGCEL |
| | stimulatory | |
| | signalling region | |
| | sequence | |
| | underlined. | |
| 80 | Pbb(trimer)/H | MQIPQAPWPVVWAVLQLGWRPGWELDSPDRPWNPPTESPALLVVTEGDNATFT |
| | 1XX pCAR | CSFSNTSESFVLNWYRMSPSNQTDKLAAFPEDRSQPGQDCRFRVTQLPNGRDF |
| | sequence. The | HMSVVRARRNDSGTYLCGAISLAPKAQIKESLRAELRVTERRAEVPTAHGGGG |
| | PD-1 sequence is | SGTPSSDAVSRLEEEMRKLQATVQELQKRLDRLEETVQAKGGGGIISFFLALT |
| | single underlined | STALLFLLFFLTLRFSVVKRGRKKLLYIFKQPFMRPVQTTQEEDGCSCRFPEE |
| | and the ITAMs of | EEGGCELRRKRSGSGEGRGSLLTCGDVEENPGPMALPVTALLLPLALLLHAEV |
| | the CD3ζ | QLQQSGGGLVQPGGSMKLSCVASGFTFSNYWMNWVRQSPEKGLEWVAEIRLKS |
| | polypeptide are | NNYATHYAESVKGRFTISRDDSKSSVYLQMNNLRAEDTGIYYCTFGNSFAYWG |
| | italicized and in | QGTTVTVSSGGGGSGGGGSGGGGSQAVVTQESALTTSPGETVTLTCRSSTGAV |
| | bold with the | TTSNYANWVQEKPDHLFTGLIGGTNNRAPGVPARFSGSLIGDKAALTITGAQT |
| | mutated residues | EDEAIYFCALWYSNHWVFGGGTKLTVLGSEAAAIEVMYPPPYLDNEKSNGTII |
| | double | HVKGKHLCPSPLFPGPSKPFWVLVVVGGVLACYSLLVTVAFIIFWVRSKRSRL |
| | underlined. | LHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGL [symbol] NELQKDKMAEA [symbol] S |
| | | EIGMKGERRRGKGHDGL [symbol] QGLSTATKDT [symbol] DALHMQALPPR |
| 81 | Pbb(trimer) CCR | MQIPQAPWPVVWAVLQLGWRPGWFLDSPDRPWNPPTFSPALLVVTEGDNATFT |
| | sequence. The | CSFSNTSESFVLNWYRMSPSNQTDKLAAFPEDRSQPGQDCRFRVTQLPNGRDF |
| | PD-1 polypeptide | HMSVVRARRNDSGTYLCGAISLAPKAQIKESLRAELRVTERRAEVPTAHGGGG |
| | sequence is in | SGTPSSDAVSRLEEEMRKLQATVQELQKRLDRLEETVQAKGGGGIISFFLALT |
| | bold and the 4- | STALLFLLFFLTLRFSVVKRGRKKLLYIFKQPFMRPVQTTQEEDGCSCRFPEE |
| | 1BB co- | EEGGCEL |
| | stimulatory | |
| | signalling region | |
| | sequence is | |
| | underlined. | |
| 82 | 44bb(trimer) /H | MAWRALHPLLLLLLLFPGSQAQSKAQVLQSVAGQTLTVRCQYPPTGSLYEKKG |
| | 1XX pCAR. The | WCKEASALVCIRLVTSSKPRTMAWTSRFTIWDDPDAGFFTVTMTDLREEDSGH |
| | NKp44 sequence | YWCRIYRPSDNSVSKSVRFYLVVSPASASTQTSWTPRDLVSSQTQTQSCVPPT |
| | is single | AGARQAPESPSTIPVPSQPQNSTLRPGPAAPIAGGGGSGTPSSDAVSRLEEEM |
| | underlined and the | RKLQATVQELQKRLDRLEETVQAKGGGGIISFFLALTSTALLFLLFFLTLRES |
| | ITAMs of the | VVKRGRKKLLYIFKQPFMRPVQTTQEEDGCSCRFPEEEEGGCELRRKRSGSGE |
| | CD3ζ polypeptide | GRGSLLTCGDVEENPGPMALPVTALLLPLALLLHAEVQLQQSGGGLVQPGGSM |
| | are italicized and | KLSCVASGFTFSNYWMNWVRQSPEKGLEWVAEIRLKSNNYATHYAESVKGRFT |
| | in bold with the | ISRDDSKSSVYLQMNNLRAEDTGIYYCTFGNSFAYWGQGTTVTVSSGGGGSGG |
| | mutated residues | GGSGGGGSQAVVTQESALTTSPGETVTLTCRSSTGAVTTSNYANWVQEKPDHL |
| | double | FTGLIGGTNNRAPGVPARFSGSLIGDKAALTITGAQTEDEAIYFCALWYSNHW |
| | underlined. | VFGGGTKLTVLGSEAAAIEVMYPPPYLDNEKSNGTIIHVKGKHLCPSPLFPGP |
| | | SKPFWVLVVVGGVLACYSLLVTVAFIIFWVRSKRSRLLHSDYMNMTPRRPGPT |
| | | RKHYQPYAPPRDFAAYRSRVKFSRSADAPAYQQGQNQLYNELNLGRREEYDVL |
| | | DKRRGRDPEMGGKPRRKNPQEGL [symbol] NELQKDKMAEA [symbol] SEIGMKGERRRGKGHDG |
| | | L [symbol] QGLSTATKDT [symbol] DALHMQALPPR |
| 83 | 44bb(trimer) CCR | MAWRALHPLLLLLLLFPGSQAQSKAQVLQSVAGQTLTVRCQYPPTGSLYEKKG |
| | sequence. The | WCKEASALVCIRLVTSSKPRTMAWTSRFTIWDDPDAGFFTVTMTDLREEDSGH |
| | NKp44 | YWCRIYRPSDNSVSKSVRFYLVVSPASASTQTSWTPRDLVSSQTQTQSCVPPT |
| | polypeptide | AGARQAPESPSTIPVPSQPQNSTLRPGPAAPIAGGGGSGTPSSDAVSRLEEEM |
| | sequence is in | RKLQATVQELQKRLDRLEETVQAKGGGGIISFFLALTSTALLFLLFFLTLRFS |
| | bold and the 4- | VVKRGRKKLLYIFKQPFMRPVQTTQEEDGCSCRFPEEEEGGCEL |
| | 1BB co- | |
| | stimulatory | |
| | signalling region | |
| | sequenced is | |
| | underlined. | |
| 84 | Human Dap12 | MGGLEPCSRLLLLPLLLAVSGLRPVQAQAQSDCSCSTVSPGVLAGIVMGDLVL |
| | full length | TVLIALAVYFLGRLVPRGRGAAEAATRKQRITETESPYQELQGQRSDVYSDLN |
| | sequence | TQRPYYK |
| 85 | Human Dap12 | YFLGRLVPRGRGAAEAATRKQRITETESPYQELQGQRSDVYSDLNTQRPYYK |
| | intracellular | |
| | domain (residues | |
| | 62-113) | |
| 86 | PD-1 protein | MQIPQAPWPVVWAVLQLGWRPGWFLDSPDRPWNPPTFSPALLVVTEGDNATFT |
| | sequence | CSFSNTSESFVLNWYRMSPSNQTDKLAAFPEDRSQPGQDCRFRVTQLPNGRDF |
| | | HMSVVRARRNDSGTYLCGAISLAPKAQIKESLRAELRVTERRAEVPTAH |
| 87 | NKp44 protein | MAWRALHPLLLLLLLFPGSQAQSKAQVLQSVAGQTLTVRCQYPPTGSLYEKKG |
| | sequence | WCKEASALVCIRLVTSSKPRTMAWTSRFTIWDDPDAGFFTVTMTDLREEDSGH |
| | | YWCRIYRPSDNSVSKSVRFYLVVSPASASTQTSWTPRDLVSSQTQTQSCVPPT |
| | | AGARQAPESPSTIPVPSQPQNSTLRPGPAAPIA |
| 88 | Dap10-CD35 | MIHLGHILFLLLLPVAAAQTTPGERSSLPAFYPGTSGSCSGCGSLSLPLLAGL |
| | (1XX) fusion | VAADAVASLLIVGAVELCARPRRSPAQEDGKVYINMPGRGRVKFSRSADAPAY |
| | protein sequence | QQGQNQLYNELNLGRREEYDVLDKRRGRDPEMGGKPRRKNPQEGLFNELQKDK |
| | Dap10 sequence | MAEAFSEIGMKGERRRGKGHDGLFQGLSTATKDTFDALHMQALPPR |
| | is bold; CD3} | |
| | (1XX) sequence is | |
| | single underlined | |
| | and ITAMs of | |
| | CD3ζ are in | |
| | italics, mutated | |
| | residues of CD35 | |
| | are double | |
| | underlined. | |
| 89 | NKG2D bb/H | atgggatggctgtgcagcggactgctgttccccgtgtcctgtctggtgctgct |
| | 1XX pCAR | gcaggtggccagctccggaaacctgtttaatcaggaggtgcagatccctctga |
| | nucleic acid | ccgagtcctactgcggcccatgtcccaagaactggatctgctacaagaacaac |
| | sequence | tgttaccagttctttgatgagtctaagaactggtatgagagccaggcctcctg |
| | | catgtctcagaatgcctccctgctgaaggtgtactctaaggaggaccaggatc |
| | | tgctgaagctggtgaagagctatcactggatgggcctggtgcacatccctaca |
| | | aacggctcctggcagtgggaggacggcagcatcctgtccccaaatctgctgac |
| | | catcatcgagatgcagaagggcgattgcgccctgtacgcctctagctttaagg |
| | | gctatatcgagaactgctccacccctaatacatacatctgtatgcagaggacc |
| | | gtgggaggaggaggaagcggaggaggaggcgacgagccaaagtcctgtgataa |
| | | gacccacacatgcccaccctgtcctatctacatctgggcaccactggcaggaa |
| | | catgcggcgtgctgctgctgagcctggtcatcaccctgtattgtaaccacaag |
| | | agaggcagaaagaagctgctgtacatcttcaagcagcccttcatgagacccgt |
| | | gcagaccacccaggaggaggacggctgcagctgcagattccccgaggaggagg |
| | | agggcggctgcgagctgagaagaaagagaagcggcagcggcgagggcagaggc |
| | | agcctgctgacctgcggcgacgtggaggagaaccccggacccatggctctccc |
| | | agtgactgccctactgcttcccctagcgcttctcctgcatgcagaggtgcagc |
| | | tgcagcagtctggaggaggcttggtgcaacctggaggatccatgaaactctcc |
| | | tgtgttgcctctggattcactttcagtaactactggatgaactgggtccgcca |
| | | gtctccagagaaggggcttgagtgggttgctgaaattagattgaaatctaata |
| | | attatgcaacacattatgcggagtctgtgaaagggaggttcaccatctcaaga |
| | | gatgattccaaaagtagtgtctacctgcaaatgaacaacttaagagctgaaga |
| | | cactggcatttattactgtacctttggtaactcctttgcttactggggccaag |
| | | ggaccacggtcaccgtctcctcaggtggaggcggttcaggcggaggtggctct |
| | | ggcggtggcggatcgcaggccgtggtcactcaggaatctgcactcaccacatc |
| | | acctggtgaaacagtcacactcacttgtcgctcaagtactggggctgttacaa |
| | | ctagtaactatgccaactgggtccaagaaaaaccagatcatttattcactggt |
| | | ctaataggtggtaccaacaaccgagcaccaggtgttcctgccagattctcagg |
| | | ctccctgattggagacaaggctgccctcaccatcacaggggcacagactgagg |
| | | atgaggcaatatatttctgtgctctatggtacagcaaccattgggtgttcggt |
| | | ggaggaaccaaactgactgtcctaggatcagaggcggccgcaattgaagttat |
| | | gtatcctcctccttacctagacaatgagaagagcaatggaaccattatccatg |
| | | tgaaagggaaacacctttgtccaagtcccctatttcccggaccttctaagccc |
| | | ttttgggtgctggtggtggttggtggagtcctggcttgctatagcttgctagt |
| | | aacagtggcctttattattttctgggtgaggagtaagaggagcaggctcctgc |
| | | acagtgactacatgaacatgactccccgccgccccgggcccacccgcaagcat |
| | | taccagccctatgccccaccacgcgacttcgcagcctatcgctccagagtgaa |
| | | gttcagcaggagcgcagacgcccccgcgtaccagcagggccagaaccagctct |
| | | ataacgagctcaatctaggacgaagagaggagtacgatgttttggacaagaga |
| | | cgtggccgggaccctgagatggggggaaagccgagaaggaagaaccctcagga |
| | | aggcctgttcaatgaactgcagaaagataagatggcggaggccttcagtgaga |
| | | ttgggatgaaaggcgagcgccggaggggcaaggggcacgatggccttttccag |
| | | ggtctcagtacagccaccaaggacaccttcgacgcccttcacatgcaggccct |
| | | gccccctcgctaa |
| 90 | NKG2Dbb(trimer)/ | atgggatggctgtgcagcggactgctgttccccgtgtcctgtctggtgctgct |
| | H 1XX pCAR | gcaggtggccagctccggaaacctgtttaatcaggaggtgcagatccctctga |
| | nucleic acid | ccgagtcctactgcggcccatgtcccaagaactggatctgctacaagaacaac |
| | sequence | tgttaccagttctttgatgagtctaagaactggtatgagagccaggcctcctg |
| | | catgtctcagaatgcctccctgctgaaggtgtactctaaggaggaccaggatc |
| | | tgctgaagctggtgaagagctatcactggatgggcctggtgcacatccctaca |
| | | aacggctcctggcagtgggaggacggcagcatcctgtccccaaatctgctgac |
| | | catcatcgagatgcagaagggcgattgcgccctgtacgcctctagctttaagg |
| | | gctatatcgagaactgctccacccctaatacatacatctgtatgcagaggacc |
| | | gtgggaggaggaggaagcggaggaggaggcgacggcggagggggcagcggcac |
| | | ccctagcagcgacgccgtgagccggctggaggaggagatgcggaagctgcagg |
| | | ccaccgtgcaggagctgcagaagcggctggaccggctggaggagaccgtgcag |
| | | gccaagggcggaggaggcatcatcagcttcttcctggccctgaccagcaccgc |
| | | cctgctgttcctgctgttcttcctgaccctgcggttcagcgtggtgaagagag |
| | | gcagaaagaagctgctgtacatcttcaagcagcccttcatgagacccgtgcag |
| | | accacccaggaggaggacggctgcagctgcagattccccgaggaggaggaggg |
| | | cggctgcgagctgagaagaaagagaagcggcagcggcgagggcagaggcagcc |
| | | tgctgacctgcggcgacgtggaggagaaccccggacccatggctctcccagtg |
| | | actgccctactgcttcccctagcgcttctcctgcatgcagaggtgcagctgca |
| | | gcagtctggaggaggcttggtgcaacctggaggatccatgaaactctcctgtg |
| | | ttgcctctggattcactttcagtaactactggatgaactgggtccgccagtct |
| | | ccagagaaggggcttgagtgggttgctgaaattagattgaaatctaataatta |
| | | tgcaacacattatgcggagtctgtgaaagggaggttcaccatctcaagagatg |
| | | attccaaaagtagtgtctacctgcaaatgaacaacttaagagctgaagacact |
| | | ggcatttattactgtacctttggtaactcctttgcttactggggccaagggac |
| | | cacggtcaccgtctcctcaggtggaggcggttcaggcggaggtggctctggcg |
| | | gtggcggatcgcaggccgtggtcactcaggaatctgcactcaccacatcacct |
| | | ggtgaaacagtcacactcacttgtcgctcaagtactggggctgttacaactag |
| | | taactatgccaactgggtccaagaaaaaccagatcatttattcactggtctaa |
| | | taggtggtaccaacaaccgagcaccaggtgttcctgccagattctcaggctcc |
| | | ctgattggagacaaggctgccctcaccatcacaggggcacagactgaggatga |
| | | ggcaatatatttctgtgctctatggtacagcaaccattgggtgttcggtggag |
| | | gaaccaaactgactgtcctaggatcagaggcggccgcaattgaagttatgtat |
| | | cctcctccttacctagacaatgagaagagcaatggaaccattatccatgtgaa |
| | | agggaaacacctttgtccaagtcccctatttcccggaccttctaagccctttt |
| | | gggtgctggtggtggttggtggagtcctggcttgctatagcttgctagtaaca |
| | | gtggcctttattattttctgggtgaggagtaagaggagcaggctcctgcacag |
| | | tgactacatgaacatgactccccgccgccccgggcccacccgcaagcattacc |
| | | agccctatgccccaccacgcgacttcgcagcctatcgctccagagtgaagttc |
| | | agcaggagcgcagacgcccccgcgtaccagcagggccagaaccagctctataa |
| | | cgagctcaatctaggacgaagagaggagtacgatgttttggacaagagacgtg |
| | | gccgggaccctgagatggggggaaagccgagaaggaagaaccctcaggaaggc |
| | | ctgttcaatgaactgcagaaagataagatggcggaggccttcagtgagattgg |
| | | gatgaaaggcgagcgccggaggggcaaggggcacgatggccttttccagggtc |
| | | tcagtacagccaccaaggacaccttcgacgcccttcacatgcaggccctgccc |
| | | cctcgctaa |
| 91 | Pbb(trimer)/H | atgcagatcccccaggctccctggcccgtggtgtgggccgtgctgcagctggg |
| | 1XX pCAR | ctggcggcctggctggttcctggacagccccgaccggccctggaaccctccca |
| | nucleic acid | ccttcagccccgccctgctggtggtgaccgagggcgacaacgccaccttcacc |
| | sequence | tgcagcttcagcaacaccagcgagagcttcgtgctgaactggtaccggatgag |
| | | ccccagcaaccagaccgacaagctggctgccttccccgaggaccggagccagc |
| | | ctggccaggactgccggttccgggtgacccagctgcccaacggacgggacttc |
| | | cacatgagcgtggtgcgggctcggaggaacgacagcggcacctacctgtgcgg |
| | | agccatcagcctggctcccaaggcccagatcaaggagagcctgcgggctgagc |
| | | tgcgggtgaccgagcggagggccgaggtgcccaccgcccacggcggagggggc |
| | | agcggcacccctagcagcgacgccgtgagccggctggaggaggagatgcggaa |
| | | gctgcaggccaccgtgcaggagctgcagaagcggctggaccggctggaggaga |
| | | ccgtgcaggccaagggcggaggaggcatcatcagcttcttcctggccctgacc |
| | | agcaccgccctgctgttcctgctgttcttcctgaccctgcggttcagcgtggt |
| | | gaagaggggcaggaagaagctgctgtacatcttcaagcagcccttcatgcggc |
| | | ctgtgcagaccacccaggaggaggacggctgcagctgccggttccccgaggag |
| | | gaggagggcggatgcgagctgagaagaaagagaagcggcagcggcgagggcag |
| | | aggcagcctgctgacctgcggcgacgtggaggagaaccccggacccatggctc |
| | | tcccagtgactgccctactgcttcccctagcgcttctcctgcatgcagaggtg |
| | | cagctgcagcagtctggaggaggcttggtgcaacctggaggatccatgaaact |
| | | ctcctgtgttgcctctggattcactttcagtaactactggatgaactgggtcc |
| | | gccagtctccagagaaggggcttgagtgggttgctgaaattagattgaaatct |
| | | aataattatgcaacacattatgcggagtctgtgaaagggaggttcaccatctc |
| | | aagagatgattccaaaagtagtgtctacctgcaaatgaacaacttaagagctg |
| | | aagacactggcatttattactgtacctttggtaactcctttgcttactggggc |
| | | caagggaccacggtcaccgtctcctcaggtggaggcggttcaggcggaggtgg |
| | | ctctggcggtggcggatcgcaggccgtggtcactcaggaatctgcactcacca |
| | | catcacctggtgaaacagtcacactcacttgtcgctcaagtactggggctgtt |
| | | acaactagtaactatgccaactgggtccaagaaaaaccagatcatttattcac |
| | | tggtctaataggtggtaccaacaaccgagcaccaggtgttcctgccagattct |
| | | caggctccctgattggagacaaggctgccctcaccatcacaggggcacagact |
| | | gaggatgaggcaatatatttctgtgctctatggtacagcaaccattgggtgtt |
| | | cggtggaggaaccaaactgactgtcctaggatcagaggcggccgcaattgaag |
| | | ttatgtatcctcctccttacctagacaatgagaagagcaatggaaccattatc |
| | | catgtgaaagggaaacacctttgtccaagtcccctatttcccggaccttctaa |
| | | gcccttttgggtgctggtggtggttggtggagtcctggcttgctatagcttgc |
| | | tagtaacagtggcctttattattttctgggtgaggagtaagaggagcaggctc |
| | | ctgcacagtgactacatgaacatgactccccgccgccccgggcccacccgcaa |
| | | gcattaccagccctatgccccaccacgcgacttcgcagcctatcgctccagag |
| | | tgaagttcagcaggagcgcagacgcccccgcgtaccagcagggccagaaccag |
| | | ctctataacgagctcaatctaggacgaagagaggagtacgatgttttggacaa |
| | | gagacgtggccgggaccctgagatggggggaaagccgagaaggaagaaccctc |
| | | aggaaggcctgttcaatgaactgcagaaagataagatggcggaggccttcagt |
| | | gagattgggatgaaaggcgagcgccggaggggcaaggggcacgatggcctttt |
| | | ccagggtctcagtacagccaccaaggacaccttcgacgcccttcacatgcagg |
| | | ccctgccccctcgctaa |
| 92 | 44bb(trimer)/H | atggcctggagggccctgcaccctctgctgctgctgctgctgctgttccccgg |
| | 1XX pCAR | cagccaggcccagagcaaggcccaggtgctgcagagcgtggctggccagaccc |
| | nucleic acid | tgaccgtgcggtgccagtaccctcccaccggcagcctgtacgagaagaagggc |
| | sequence | tggtgcaaggaggccagcgccctggtgtgcatccggctggtgaccagcagcaa |
| | | gcctcggacaatggcctggaccagccggttcaccatctgggacgaccccgacg |
| | | ctggcttcttcaccgtgaccatgaccgacctgcgggaggaggacagcggccac |
| | | tactggtgccggatctacaggcccagcgacaacagcgtgagcaagagcgtgcg |
| | | gttctacctggtggtgagccctgccagcgccagcacccagaccagctggaccc |
| | | ctcgggacctggtgagcagccagacccagacccagagctgcgtgcctcccacc |
| | | gccggagcccggcaggctcccgagagccccagcaccatccccgtgcccagcca |
| | | gccccagaacagcaccctgcggcctggacccgctgcccctatcgccggcggag |
| | | ggggcagcggcacccctagcagcgacgccgtgagccggctggaggaggagatg |
| | | cggaagctgcaggccaccgtgcaggagctgcagaagcggctggaccggctgga |
| | | ggagaccgtgcaggccaagggcggaggaggcatcatcagcttcttcctggccc |
| | | tgaccagcaccgccctgctgttcctgctgttcttcctgaccctgcggttcagc |
| | | gtggtgaagaggggcaggaagaagctgctgtacatcttcaagcagcccttcat |
| | | gcggcctgtgcagaccacccaggaggaggacggctgcagctgccggttccccg |
| | | aggaggaggagggcggatgcgagctgagaagaaagagaagcggcagcggcgag |
| | | ggcagaggcagcctgctgacctgcggcgacgtggaggagaaccccggacccat |
| | | ggctctcccagtgactgccctactgcttcccctagcgcttctcctgcatgcag |
| | | aggtgcagctgcagcagtctggaggaggcttggtgcaacctggaggatccatg |
| | | aaactctcctgtgttgcctctggattcactttcagtaactactggatgaactg |
| | | ggtccgccagtctccagagaaggggcttgagtgggttgctgaaattagattga |
| | | aatctaataattatgcaacacattatgcggagtctgtgaaagggaggttcacc |
| | | atctcaagagatgattccaaaagtagtgtctacctgcaaatgaacaacttaag |
| | | agctgaagacactggcatttattactgtacctttggtaactcctttgcttact |
| | | ggggccaagggaccacggtcaccgtctcctcaggtggaggcggttcaggcgga |
| | | ggtggctctggcggtggcggatcgcaggccgtggtcactcaggaatctgcact |
| | | caccacatcacctggtgaaacagtcacactcacttgtcgctcaagtactgggg |
| | | ctgttacaactagtaactatgccaactgggtccaagaaaaaccagatcattta |
| | | ttcactggtctaataggtggtaccaacaaccgagcaccaggtgttcctgccag |
| | | attctcaggctccctgattggagacaaggctgccctcaccatcacaggggcac |
| | | agactgaggatgaggcaatatatttctgtgctctatggtacagcaaccattgg |
| | | gtgttcggtggaggaaccaaactgactgtcctaggatcagaggcggccgcaat |
| | | tgaagttatgtatcctcctccttacctagacaatgagaagagcaatggaacca |
| | | ttatccatgtgaaagggaaacacctttgtccaagtcccctatttcccggacct |
| | | tctaagcccttttgggtgctggtggtggttggtggagtcctggcttgctatag |
| | | cttgctagtaacagtggcctttattattttctgggtgaggagtaagaggagca |
| | | ggctcctgcacagtgactacatgaacatgactccccgccgccccgggcccacc |
| | | cgcaagcattaccagccctatgccccaccacgcgacttcgcagcctatcgctc |
| | | cagagtgaagttcagcaggagcgcagacgcccccgcgtaccagcagggccaga |
| | | accagctctataacgagctcaatctaggacgaagagaggagtacgatgttttg |
| | | gacaagagacgtggccgggaccctgagatggggggaaagccgagaaggaagaa |
| | | ccctcaggaaggcctgttcaatgaactgcagaaagataagatggcggaggcct |
| | | tcagtgagattgggatgaaaggcgagcgccggaggggcaaggggcacgatggc |
| | | cttttccagggtctcagtacagccaccaaggacaccttcgacgcccttcacat |
| | | gcaggccctgccccctcgctaa |

### 7. EXAMPLES

### 7.1. Methods Used in Studies of Examples 1-4

Generation of H2 1XX CAR constructs. The H2 1XX CAR is a modified version of the second generation H2 CAR described in Wilkie et al., J. Immunol. 180:4901-9 (2008) and WO2020/183158, both of which are incorporated by reference herein in their entireties. Briefly, the two tyrosine (Y) residues in each of the two distal immunoreceptor tyrosine-based activation motifs (ITAMs) of the CD3ζ polypeptide in the H2 CAR are mutated to phenylalanine (F) residues. The resulting H2 1XX CAR comprises a CD3ζ polypeptide with a functional ITAM1 and non-functional ITAMs 2 and 3 (“1XX”). The H2 1XX CAR has the amino acid sequence of SEQ ID NO: 59.

Generation of TBB/H 1XX pCAR constructs. The TBB/H 1XX pCAR is a modified version of the TBB/H pCAR described in WO2020/183158 which is incorporated by reference herein in its entirety. Briefly, the two tyrosine (Y) residues in each of the two distal immunoreceptor tyrosine-based activation motifs (ITAMs) of the CD3ζ polypeptide in the H2 CAR of the TBB/H pCAR are mutated to phenylalanine (F) residues. The resulting TBB/H 1XX pCAR comprises a CAR with a CD3ζ polypeptide comprising a functional ITAM1 and non-functional ITAMs 2 and 3 (“1XX”). The TBB/H 1XX pCAR has the amino acid sequence of SEQ ID NO: 23. Deletion of the two distal ITAMs (ITAM2 and ITAM3) were made to generate TBB/H-1, a pCAR with a CAR comprising a truncated CD3ζ polypeptide with only one ITAM (ITAM1). The TBB/H-1 pCAR has the amino acid sequence of SEQ ID NO: 26. To generate the TBB/H-1Δ3, TBB/H-1Δ6 and TBB/H-1Δ9 pCARs, deletions were made to remove 3, 6, and 9 amino acid residues upstream of the single remaining ITAM (ITAM1) of the TBB/H-1 pCAR. The TBB/H-1Δ3, TBB/H-1Δ6 and TBB/H-1Δ9 pCARs have the amino acid sequences of SEQ ID NOs: 27-29, respectively. To generate the TBB/H-1Δ9 YRS pCAR, the TBB/H-1Δ9 pCAR was further modified to delete the YRS motif at the C-terminal end of the CD28 co-stimulatory signalling region. The TBB/H-1Δ9 YRS pCAR has the amino acid sequence of SEQ ID NO: 30.

Generation of alternative TBBIH pCARs. Activation domains from FcεR1γ or DAP12 were substituted for the CD3ζ domain in the TBB/H pCAR to generate TBB/H-FcεR1γ pCAR or TBB/H-DAP12 pCAR, respectively. The FcεR1γ pCAR has the amino acid sequence of SEQ ID NO: 54. The DAP12 pCAR has the amino acid sequence of SEQ ID NO: 56.

Generation of NKG2D_bb/H 1XX pCAR constructs. The NKG2D_bb/H 1XX pCAR utilizes the same MUC1-targeting second-generation CAR as the TBB/H 1XX pCAR described above. The CCR in the NKG2D_bb/H 1XX pCAR comprises two extracellular NKG2D domains that are each fused to a transmembrane domain and an intracellular 4-1BB co-stimulatory domain. To generate this construct, a synthetic cDNA encoding for the CCR was substituted for that in TBB/H 1XX. The resulting NKG2D_bb/H 1XX pCAR comprises a MUC1-targeting CAR with a CD3ζ polypeptide comprising a functional ITAM1 and non-functional ITAMs 2 and 3 (“1XX”) and a CCR that binds NKG2D ligands. The NKG2D_bb/H 1XX pCAR has the amino acid sequence of SEQ ID NO: 76 and the nucleic acid sequence of SEQ ID NO: 89.

Generation of NKG2Dbb(trimer)/H 1XX pCAR constructs. The NKG2Dbb(trimer)/H 1XX pCAR utilizes the same MUC1-targeting second-generation CAR as the TBB/H 1XX pCAR described above. The CCR in the NKG2Dbb(trimer)/H 1XX pCAR comprises three extracellular NKG2D domains that are each fused via a trimerization domain to a transmembrane domain and an intracellular 4-1BB co-stimulatory domain. To generate this construct, a synthetic cDNA encoding for the CCR was substituted for that in TBB/H 1XX. The resulting NKG2Dbb(trimer)/H 1XX pCAR comprises a CAR with a CD3ζ polypeptide comprising a functional ITAM1 and non-functional ITAMs 2 and 3 (“1XX”). The NKG2Dbb(trimer)/H 1XX pCAR has the amino acid sequence of SEQ ID NO: 78 and the nucleic acid sequence of SEQ ID NO: 90.

Generation of Pbb(trimer)/H 1XX pCAR constructs. The Pbb(trimer)/H 1XX pCAR utilizes the same MUC1-targeting second-generation CAR as the TBB/H 1XX pCAR described above. The CCR in the Pbb(trimer)/H 1XX pCAR comprises three extracellular PD-1 domains that are each fused to a transmembrane domain and an intracellular 4-1BB co-stimulatory domain. To generate this construct, a synthetic cDNA encoding for the CCR was substituted for that in TBB/H 1XX. The resulting Pbb(trimer)/H 1XX pCAR comprises a CAR with a CD3ζ polypeptide comprising a functional ITAM1 and non-functional ITAMs 2 and 3 (“1XX”). The Pbb(trimer)/H 1XX pCAR has the amino acid sequence of SEQ ID NO: 80 and the nucleic acid sequence of SEQ ID NO: 91.

Generation of 44bb(trimer)/H 1XX pCAR constructs. The Pbb(trimer)/H 1XX pCAR utilizes the same MUC1-targeting second-generation CAR as the TBB/H 1XX pCAR described above. The CCR in the 44bb(trimer)/H 1XX pCAR comprises three extracellular NKp44 domains that are each fused to a transmembrane domain and an intracellular 4-1BB co-stimulatory domain. To generate this construct, a synthetic cDNA encoding for the CCR was substituted for that in TBB/H 1XX. The resulting 44bb(trimer)/H 1XX pCAR comprises a CAR with a CD3ζ polypeptide comprising a functional ITAM1 and non-functional ITAMs 2 and 3 (“1XX”). The 44bb(trimer)/H 1XX pCAR has the amino acid sequence of SEQ ID NO: 82 and the nucleic acid sequence of SEQ ID NO: 92.

T cell culture and transduction. Peripheral blood mononuclear cells (PBMCs) were isolated from healthy donor blood samples by density gradient centrifugation using Ficoll-Paque. PBMCs were activated with 5 μg/mL phytohemagglutinin (PHA)-L and cultured in RPMI with GlutaMax supplemented with 5% human AB serum for 24-48 hrs. Cells were supplemented with IL-2 (100U/ml) 24 hours prior to T cell retroviral transduction of CAR constructs. CAR T cells were expanded in RPMI with GlutaMax supplemented with 5% human AB serum plus IL-2 (100U/ml) for 10-14 days before use in functional assays. Transduced cells are identified according to the CAR construct with which they were transduced (e.g. H2 T cells, H2 1XX T cells, TBB/H 1XX T cells).

Repeated antigen stimulation assays. Efficacy of CAR T cells was tested in repeated antigen stimulation assays. BXPC3 tumour cells and CAR T cells were cocultured at an effector target ratio of 1:1 for 72-96 hours. T cells were removed, resuspended in fresh medium, counted, and added to a new BXPC3 tumour monolayer. Residual tumour cell viability was measured by MTT assay after each restimulation. CAR T cells were added to fresh monolayer until tumour cell monolayer reached 80% viability compared to untreated monolayer.

In vivo tumour monitoring by bioluminescence imaging. NSG mice were 6-10 weeks old when used for in vivo experiments and were allocated to experimental groups based on similar average tumour burden prior to treatment. BXPC3 tumor cells were transduced with an SFG retroviral vector that co-expressed firefly luciferase (ffLuc) and dsTomato red fluorescent protein (RFP) and were purified by flow sorting prior to engraftment in vivo. 1×10⁵ BXPC3 cells were inoculated via the intraperitoneal (i.p.) route and tumours were allowed to establish for 11 days. At that time, CAR T-cells or PBS as control were administered using the i.p. route at a dose of 1×10⁷ cells. The CD19-specific F28z CAR contains an FMC63 scFv coupled to a CD28 spacer, transmembrane domain and endodomain followed by a CD3ζ endodomain and was used as an additional negative control. Tumour burden was monitored by serial bioluminescence imaging. This was performed using an IVIS Spectrum Imaging platform (PerkinElmer) with Living Image software. To monitor tumour status, mice were injected i.p. with D-luciferin (150 mg/kg) and imaged under isoflurane anaesthesia after 20 minutes. In all experiments, animals were inspected daily and weighed weekly.

In vivo tumour monitoring by tumour volume measurements. NSG mice were 6-10 weeks old when used for experiments and were allocated to experimental groups based on similar average tumour burden prior to treatment. BXPC3 cells (1×10⁵ cells) were established subcutaneously in NSG mice for 17 days prior to intravenous administration of 10 million of the indicated T-cell populations, or PBS as control. Tumour volume was monitored using calipers using the formula π/6×W×W×L where W=width; L-length.

### 7.2. Methods Used in Studies of Example 5

Cell lines and tissue culture. BXPC3 and REN tumour cell lines were used in these studies. Tumour cell lines were grown in R10 or D10 medium, respectively, comprising RPMI or DMEM supplemented with 10% FBS and GlutaMax. PG13 and 293VEC-RD114 cells retroviral packaging cells were maintained in D10. Cells were maintained at 37° C. in a humidified atmosphere of 5% CO₂. Cell lines were validated by STR typing and were routinely monitored for mycoplasma contamination. Where indicated, cell lines were engineered to express RFP/ffLuc.

Human study oversight. Blood samples were obtained from healthy male and female volunteers aged between 18-65 years old.

### Generation of Alternative NKG2D Adaptor CARs.

All CARs were constructed by gene synthesis and cloning (Genscript, Hong Kong, China and Leiden, The Netherlands) using human codon optimized sequences and ligation of digested DNA fragments as appropriate.

To construct SFG NKG2D/Dap10-12, a synthetic DNA was constructed that encoded for the following elements: full length human Dap10-Dap12 endodomain (UniProt amino acids 62-113)-RRKR (furin cleavage site)-[serine-glycine]2 linker-Porcine Teschovirus (P2A) ribosomal skip peptide-NKG2D including stop codon-XhoI restriction site. This sequence was flanked on the 5′ side by sequences from the unique AgeI to NcoI restriction sites within SFG (NcoI site coincides with start codon in the synthetic fragment, thereby disrupting this site). Following digestion by AgeI and XhoI, these sequences were ligated with SFG that had been similarly digested. The NKG2D/Dap10-12 adaptor CAR has the nucleic acid sequence of SEQ ID NO: 66. The NKG2D/Dap10-12 adaptor CAR has the amino acid sequence of SEQ ID NO: 67.

To generate SFG NKG2D, the SFG NKG2D/Dap10-12 construct was digested using NcoI and XhoI (releases the NKG2D cDNA sequence). This fragment was ligated with SFG that had been similarly digested. NKG2D has the amino acid sequence of SEQ ID NO: 75.

To generate SFG NKG2D-CD3ζ, a synthetic cDNA encoding start codon (ATG)-CD3ζ endodomain-full length NKG2D-stop codon-XhoI restriction site was synthesized as above and flanked on the 5′ side by sequences from the unique AgeI-NcoI restriction sites within SFG as for SFG NKG2D/Dap10-12. This fragment was inserted into SFG following digestion with AgeI and XhoI. The NKG2D-CD3ζ CAR has the nucleic acid sequence of SEQ ID NO: 68. The NKG2D-CD3ζ CAR has the amino acid sequence of SEQ ID NO: 69.

To generate SFG NKG2D/Dap10-CD3ζ, a synthetic cDNA encoding full length human Dap10-CD3ζ endodomain-RRKR (furin cleavage site)-[serine-glycine]2 linker-Porcine Teschovirus (P2A) ribosomal skip peptide-full length NKG2D-stop codon-XhoI restriction site was synthesized and flanked on the 5′ side by sequences from the unique AgeI-NcoI restriction sites within SFG as for SFG NKG2D/Dap10-CD3ζ. This was inserted into SFG following digestion with AgeI and XhoI. The NKG2D/Dap10-CD3ζ adaptor CAR has the amino acid sequence of SEQ ID NO: 72 and the nucleic acid sequence of SEQ ID NO: 71.

The SFG NKG2D/Dap10-CD3ζ (1XX) adaptor CAR was generated by insertional mutagenesis, converting all four tyrosines in CD3ζ ITAMs 2 and 3 within SFG NKG2D/Dap10-CD3ζ to phenylalanine. The CD3ζ 1XX has the amino acid sequence of SEQ ID NO: 42 (variant 2) or SEQ ID NO 43 (variant 1). The NKG2D/Dap10-CD3ζ 1XX adaptor CAR has the nucleic acid sequence of SEQ ID NO: 73. The NKG2D/Dap10-CD3ζ 1XX adaptor CAR has the amino acid sequence of SEQ ID NO: 74.

T cell culture and transduction for adaptor CAR. Viral vector was prepared as described using PG13 cell lines, 293VEC-RD114 cells or by triple transfection of 293T cells. For the latter, 1.65×10⁶ low passage 293T cells in 11 mL IMDM+10% FBS were evenly distributed in a 10 cm plate. After 8-24h, GeneJuice (30 μL) was added to 470 μL IMDM (no serum) and mixed gently. After incubation for 5 minutes at room temperature, 3.125 μg RD114 plasmid, 4.6875 μg pEQ-Pam3 plasmid and 4.6875 μg SFG vector of interest were added to the GeneJuice/medium mixture, mixed gently and incubated for 15 minutes at room temperature. The transfection mixture was added dropwise to the plate and gently swirled to ensure even distribution. After incubation for 48 h at 37° C., 5% CO₂, medium was removed for snap freezing using an ethanol dry ice bath and replaced. After a further 24h, this procedure was repeated. Frozen virus was stored in aliquots at −80° C. Retroviral transduction of phytohemagglutinin-activated T-cells was performed using RetroNectin-coated plasticware.

Flow cytometry analysis. All cell staining reactions were performed on ice. For intracellular antigen detection, cells were stained with a fixable Live/Dead dye before being stained for surface proteins for 30 min on ice. Intracellular staining was performed by fixation with 0.01% formaldehyde followed by permeabilization using PBS+0.5% bovine serum albumin+0.1% saponin. Cells were subsequently stained for intracellular proteins for 30 min at 4° C. All gates were set using isotype control antibodies or fluorescence minus one controls. Where necessary, a viability stain was included and nonspecific binding of the antibodies was limited by using an appropriate Fc blocking reagent prior to the staining steps. Flow cytometry was performed using a FACSCalibur cytometer with CellQuest Pro software or BD LSRFortessa cytometer with BD FACSDiva software and data was analyzed using FlowJo, LLC.

Enzyme-linked immunosorbent assay. Supernatants collected from co-culture of tumour cells with CAR T-cells were analyzed using a human IFN-γ or human IL2 enzyme-linked immunosorbent assay (ELISA) as described by the manufacturers, with a limit of sensitivity of 1 pg/mL. In pooled re-stimulation assays, cytokine production was set to zero in each cycle after T-cell culture.

Cytotoxicity assays. Tumour cells were incubated with T-cells at specified effector to target (E:T) ratios. In the case of adherent targets, residual tumour cell viability was quantified using an MTT assay. After removal of the supernatant and residual T-cells, MTT was added at 500 μg/mL in D10 medium for 40 minutes at 37° C. and 5% CO₂. Formazan crystals were resuspended in DMSO and absorbance was measured at 560 nm. Alternatively, tumour cell viability was monitored by luciferase assays. D-luciferin was added at 150 mg/mL immediately prior to luminescence reading. In each case, tumour cell viability was calculated as follows: (absorbance or luminescence of tumour cells cultured with T cells/absorbance or luminescence of untreated monolayer alone)×100%. In pooled re-stimulation assays, tumour viability was set to 100% in each cycle after T-cell cultures failed.

Tumour re-stimulation assays. Transduced T-cells were co-cultured with tumour cell lines in the absence of exogenous cytokine support. Tumour restimulation assays were performed by addition of 5×10⁴ CAR T-cells to an equal number of tumour cells. Every 3-4 days, T-cells were transferred to a fresh monolayer of 5×10⁴ tumour cells. Re-stimulations were continued until T-cells were no longer recovered or tumour cell destruction fell below 70%. Supernatant was harvested after 24 h for cytokine analysis while tumour cell viability was determined after 24 h or 72 h by MTT or luciferase assay. If T-cells could not be re-stimulated, tumour viability was set to 100% and cytokine production was set to zero.

### 7.3. Example 1: Assessing Anti-Tumour Activity of MUC1 Targeting pCAR-T Cells and MUC1 Targeting Second-Generation CAR-T Cells with Modified CD3ζ Signalling Domains

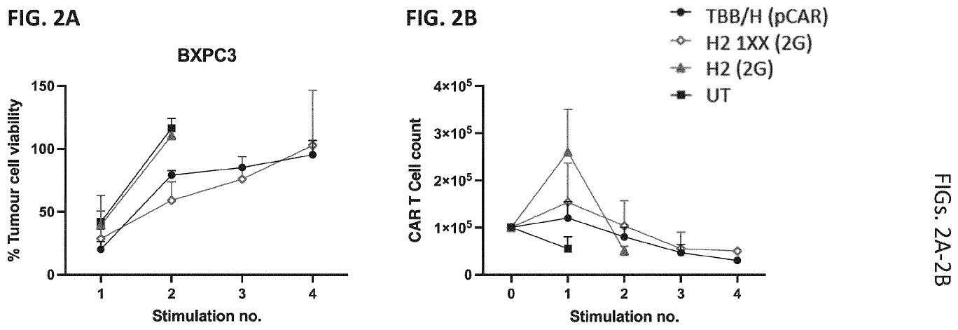

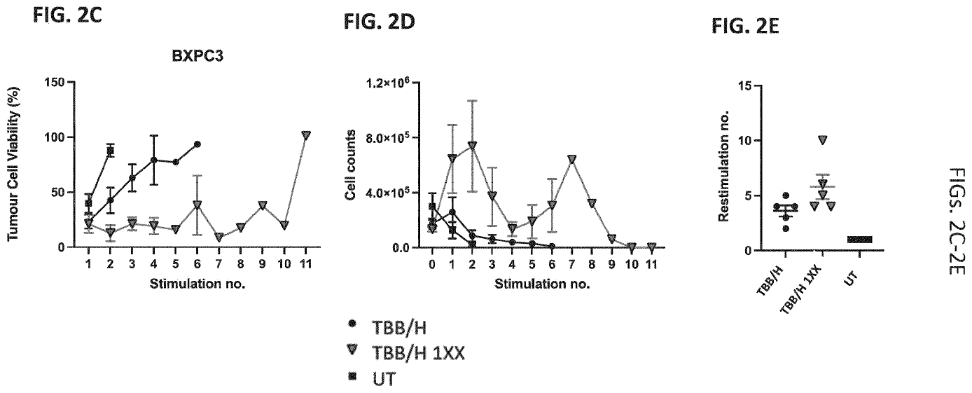

T-cells that express the CARs or pCARs of FIG. 1A were subjected to successive rounds of antigen stimulation as described above. Briefly, untransduced (UT) T-cells and T-cells that express a CAR (H2; H2 1XX) or pCAR (TBB/H; TBB/H 1XX) were co-cultured in vitro for 72 hours with BXPC3 tumour cells at an effector:target ratio (T-cells:tumour cells) of 1:1. T cells were removed, resuspended in fresh medium, counted, and added to a new BXPC3 tumour monolayer. Residual tumour cell viability was quantified by MTT assay. Results in FIGS. 2A-2E show the percentage survival of BXPC3 tumour cells following restimulation (FIGS. 2A and 2C), the T-cell counts following restimulation (FIGS. 2B and 2D), and restimulation capability of the T-cells on BXPC3 tumour monolayers (FIG. 2E).

H2 1XX CAR T-cells were assessed for anti-tumour activity and restimulation capability on tumour cells. As shown in FIGS. 2A-2B, the H2 1XX CAR T-cells (open diamond) demonstrated anti-tumour activity for more restimulation cycles compared to the H2 CART cells, with equivalence to TBB/H CAR T-cells.

TBB/H 1XX CAR-T cells were assessed for anti-tumour activity and restimulation capability on tumour cells. Results in FIGS. 2C-2E show that TBB/H 1XX T-cells (inverted triangle) had better restimulation capability on BXPC3 tumour monolayers with increased proliferative capacity as compared to TBB/H-T cells or untransduced T-cells (UT). The data demonstrate that a single functional ITAM (proximal) in the CD3ζ signalling domain is sufficient for potent anti-tumour activity in the context of a pCAR. The data further demonstrates that T cells expressing the TBB/H 1XX pCAR (with only a single functional ITAM in the CD3ζ signalling domain) have improved restimulation capability and increased proliferative capacity on BXPC3 tumour monolayers compared to T-cells expressing the TBB/H pCAR (with three functional ITAMs in the CD3ζ signalling domain).

### 7.4. Example 2: Assessing Anti-Tumour Activity of MUC1 Targeting pCAR T-Cells with Truncated CD34 Signalling Domains

In order to evaluate whether T-cells expressing a pCAR with a CD3ζ signalling domain comprising a functional ITAM1 and nonfunctional ITAM2 and ITAM3 are as effective at killing tumour cells as T-cells expressing a pCAR with a CD3ζ signalling domain that lacks ITAM2 and ITAM3, various truncated versions of the TBB/H pCAR were generated (TBB/H-1, TBB/H-1Δ3, TBB/H-1Δ6, TBB/H-1Δ9, and TBB/H-1Δ9 YRS, see Methods and FIG. 1B).

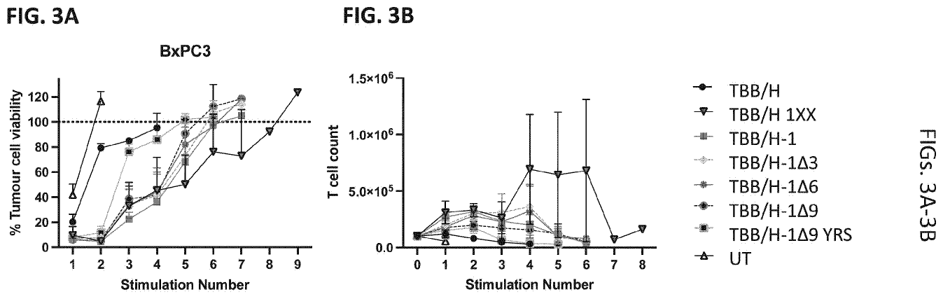

Results of restimulation assays performed with pCAR T-cells expressing a truncated CD3ζ signalling domain demonstrate that removal of the two distal ITAMs of CD3ζ hindered the anti-tumour efficacy of the T-cells compared to TBB/H 1XX pCAR T-cells (FIG. 3A). Results of the T-cell count following each stimulation cycle further demonstrate that the pCAR T-cells expressing truncated CD3ζ had reduced proliferative capacity compared to TBB/H 1XX pCAR T-cells (FIG. 3B). The data demonstrate that while one single proximal ITAM in the CD3ζ signalling domain was sufficient for potent anti-tumour activity, inclusion of the two distal non-functional ITAMs improved anti-tumour efficacy and proliferative capacity compared to T cells expressing a truncated CD3ζ signalling domain (ITAM1 only).

### 7.5. Example 3: Assessing Anti-Tumour Activity of MUC1 Targeting pCAR T-Cells with Alternative Intracellular Signalling Domains

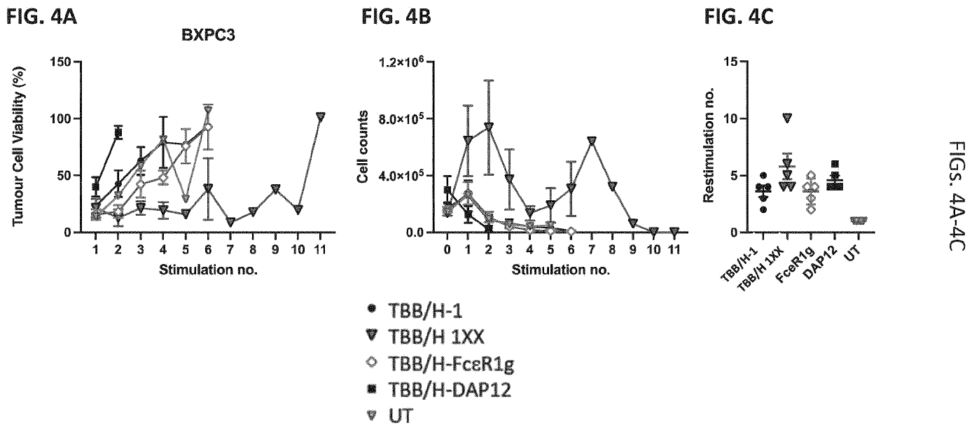

In order to assess whether pCAR T-cells that express signalling domains other than CD3ζ have anti-tumour activity and proliferation potential similar to TBB/H 1XX pCAR T-cells, pCARs with alternative signalling domains from DAP12 and FcεR1γ were generated (see Methods and FIG. 1C). As shown in FIGS. 4A-4C, TBB/H 1XX pCAR T-cells (inverted triangle) demonstrated superior anti-tumour activity (FIG. 4A), proliferation potential (FIG. 4B), and restimulation capacity (FIG. 4C) compared to pCAR T-cells expressing the DAP12 or FcεR1γ signalling domain. Equivalents and incorporation by reference.

### 7.6. Example 4: Evaluating Anti-Tumour Efficacy of MUC1 Targeting pCAR T-Cells with Alternative Chimeric Co-Stimulatory Receptors (CCRs) In Vivo

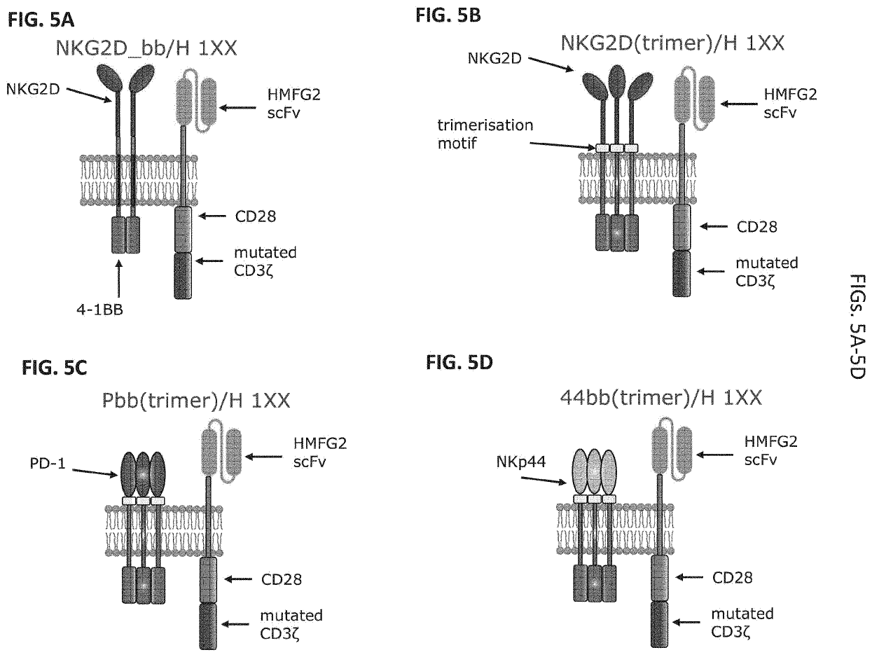

In order to evaluate efficacy of MUC1 targeting pCAR T-cells that express alternative chimeric co-stimulatory receptors (CCRs) on pancreatic tumours in vivo, pCARs with alternative CCRs were generated and T-cells expressing the constructs were produced and injected into mice bearing firefly luciferase (ffLuc)-expressing BXPC3 tumour xenografts (see Methods and FIGS. 5A-5D). Tumour growth was subsequently monitored by bioluminescence imaging.

Briefly, 1×10⁷ pCAR T-cells were injected into BXPC3 xenograft-containing mice via intraperitoneal injection 11 days after tumour inoculation. Either PBS or a similar number of F28z (CD19-specific control) CAR T cells were injected in control mice.

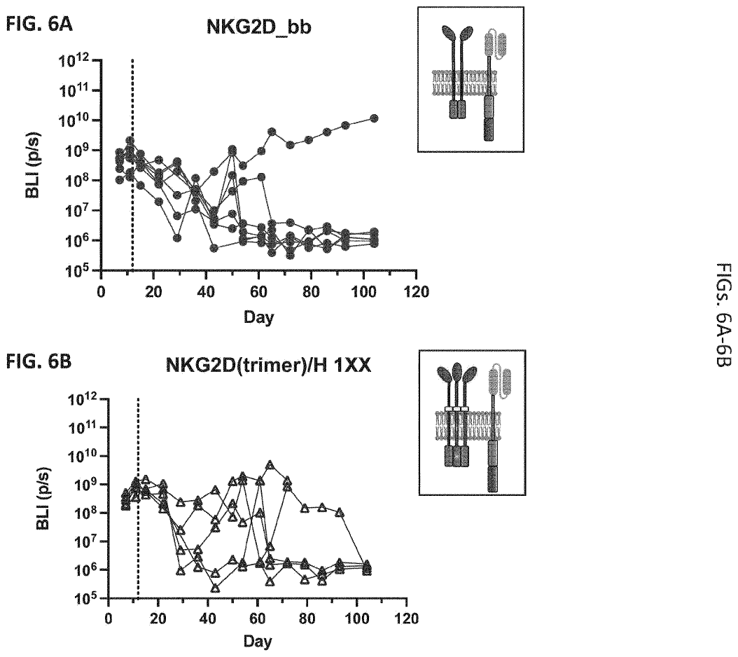

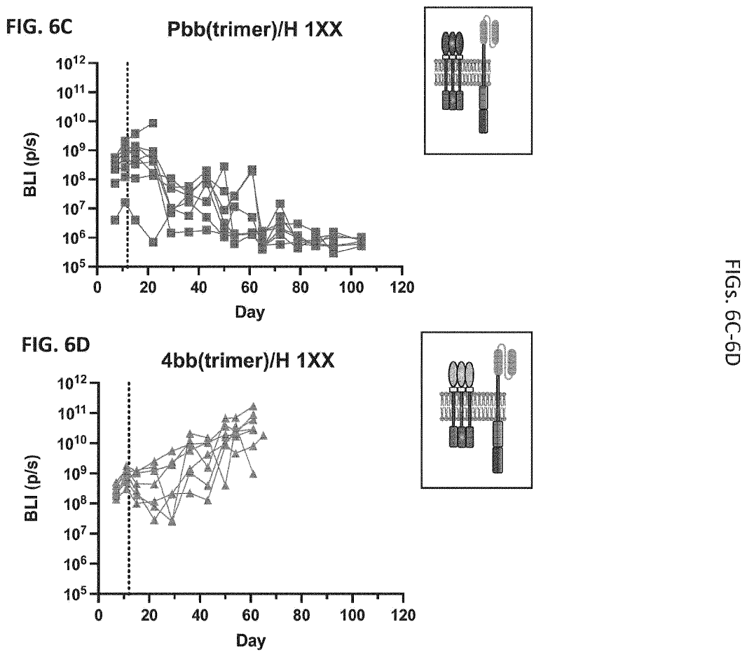

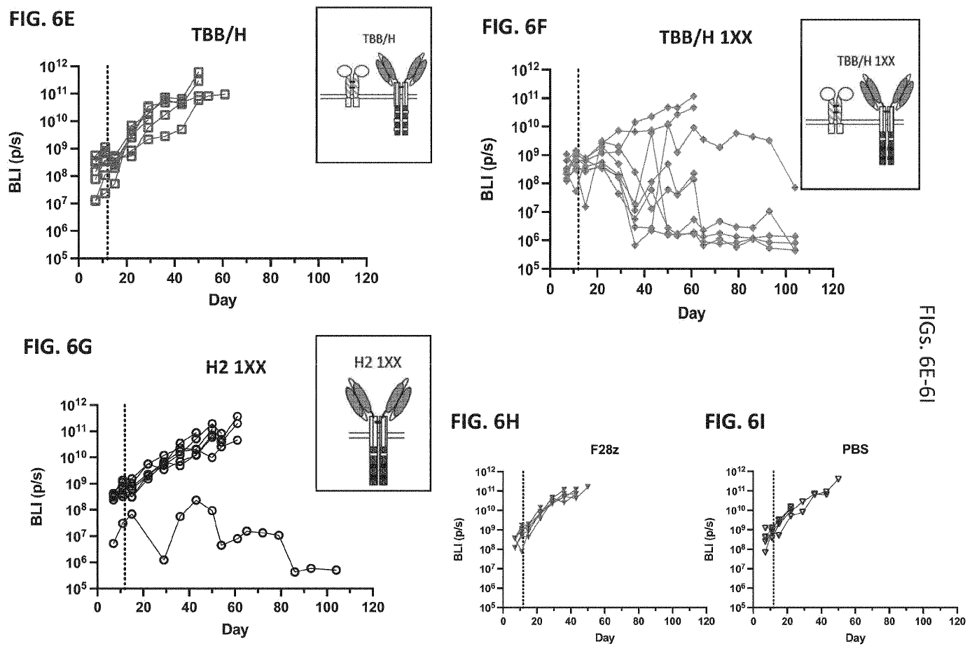

Results shown in FIGS. 6A-6I demonstrate that treatment with NKG2D_bb/H 1XX pCAR-T cells resulted in reduced tumour growth in nearly all mice tested (FIG. 6A). Similar results were observed for mice treated with NKG2Dbb(trimer)/H 1XX and Pbb(trimer)/H 1XX pCAR T-cells (FIGS. 6B and 6C, respectively). The results were comparable to those of mice treated with TBB/H 1XX pCAR T-cells (FIG. 6F). By contrast, mice treated with 44bb(trimer)/H 1XX (FIG. 6D), TBB/H (FIG. 6E) pCAR T-cells, and H2 1XX CAR T-cells (FIG. 6G) showed tumour growth similar to control groups (FIGS. 6H and 6I). The data indicate that pCARs with alternative CCRs (e.g., NKG2D or PD-1 as targeting domains) inhibit tumour growth in this model (FIGS. 6B and 6C). Further, the data show that modifying the CD3ζ activation domain of the pCAR so that it has only a single functional ITAM (ITAM1), results in enhanced anti-tumour activity (comparing results in FIG. 6E and FIG. 6F).

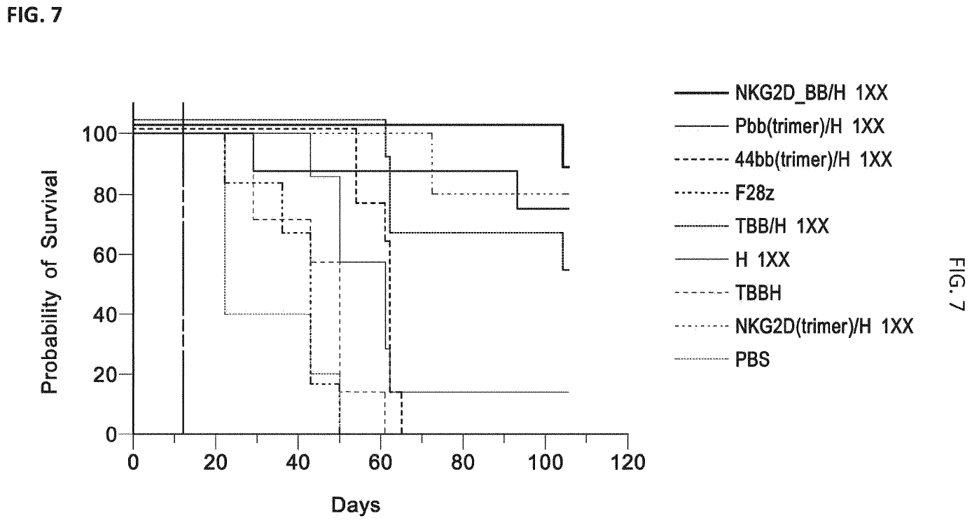

FIG. 7 shows a survival curve of the BXPC3 tumour xenograft-containing mice treated with the CAR or pCAR T-cells as indicated in FIGS. 6A-6I. Consistent with the BLI assessment of tumour growth shown in FIGS. 6A-6I, mice treated with NKG2D_bb/H 1XX, NKG2Dbb(trimer)/H 1XX and Pbb(trimer)/H 1XX pCAR T-cells survived for longer compared to mice treated with 44bb(trimer)/H 1XX, TBB/H 1XX, H 1XX, or TBBH pCAR T-cells.

The results demonstrate NKG2D_bb/H 1XX, NKG2Dbb(trimer)/H 1XX and Pbb(trimer)/H 1XX pCARs exhibit superior anti-tumour efficacy in the BXPC3 mouse model compared to pCARs comprising CCRs that target NKp44 ligands or ErbB (e.g., 44bb(trimer)/H 1XX, TBB/H, TBB/H 1XX). The combination of 41BB as a co-stimulation domain in the CCR and a CD3ζ (1XX) mutated activation domain in the CAR appears to provide balanced activating and costimulatory signals inducing robust cytotoxicity whilst maintaining persistence, two characteristics which are essential when targeting solid tumours.

### 7.7. Example 5: Evaluating the Role of CAR Structure in Targeting NKG2D Ligands

In order to evaluate alternative activation signalling domains in adaptor CARs that target NKG2D ligands, three adaptor CARs were engineered. Each adaptor CAR comprised two polypeptides: (i) an adaptor protein that was a fusion of a T-cell activation signalling domain and a Dap10 co-stimulatory signalling domain; and (ii) an NKG2D targeting moiety (binding element). The adaptor CARs differed in the activation signalling domain of the adaptor protein: Dap12, CD3ζ, or CD3ζ (1XX).

Anti-tumour activity and expansion ability were assessed and compared between adaptor CARs with an activation signalling domain comprising three ITAMs per CAR monomer (CD3ζ) and adaptor CARs with an activation signalling domain comprising a single ITAM (Dap12 and CD3ζ (1XX)). The CD3ζ 1XX domain was generated by substituting each of the tyrosine (Y) residues in ITAM2 and ITAM3 of wild-type CD3ζ to phenylalanine (F) residues, resulting in a CD3ζ domain with a single functional ITAM: ITAM1.

In addition, the role of exogenously expressed Dap10 in improving cell surface expression of CARs targeting NKG2D ligands was investigated by comparing cell surface expression of the adaptor CARs described above with cell surface expression of a linear 2G CAR comprising an NKG2D binding element (targeting moiety) and a CD3ζ activation signalling domain.

Schematics of the adaptor CARs (NKG2D/Dap10-12; NKG2D/Dap10-CD3ζ; NKG2D/Dap10-CD3ζ (1XX)) and the linear CAR (NKG2D-CD3ζ) that were evaluated in these studies are shown in FIG. 8.

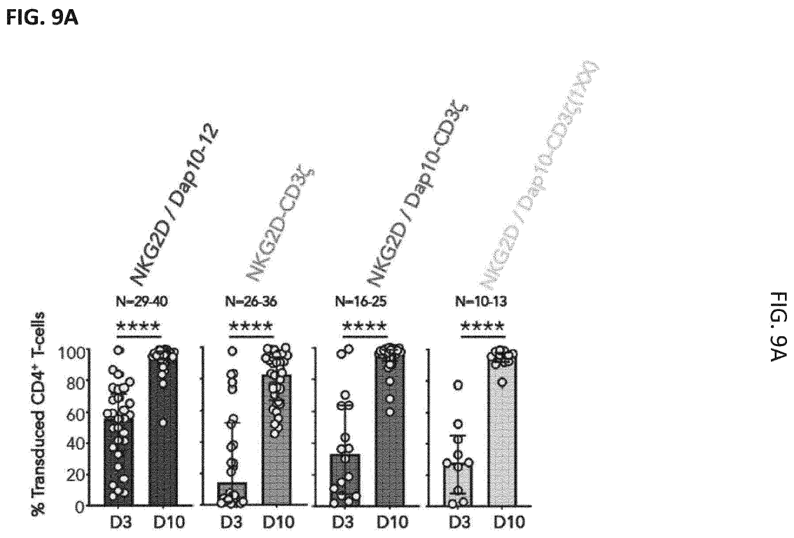

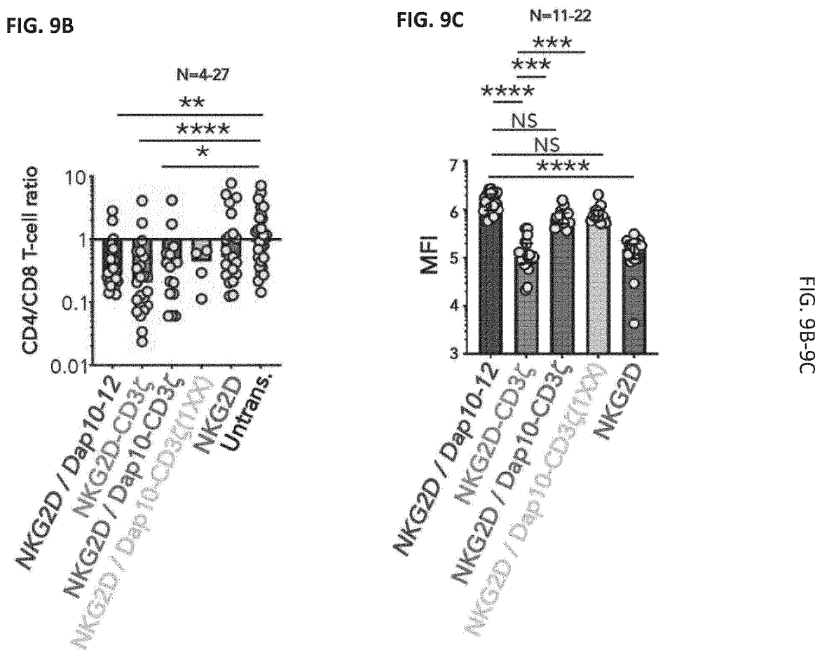

High efficiency expression of each adaptor CAR (NKG2D/Dap10-12; NKG2D/Dap10-CD3ζ; NKG2D/Dap10-CD3ζ (1XX)) and linear CAR (NKG2D-CD3ζ) was achieved in transduced T-cells, with progressive enrichment of transduced cells between day 3 and day 10 (FIG. 9A). Cultures of transduced T-cells were skewed toward the CD8⁺ T-cell subset (FIG. 9B).

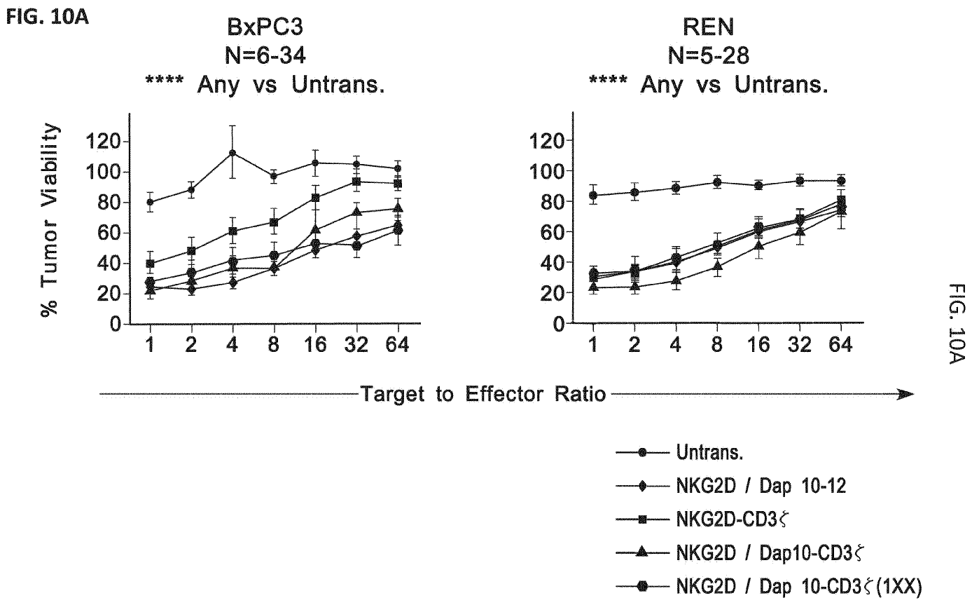

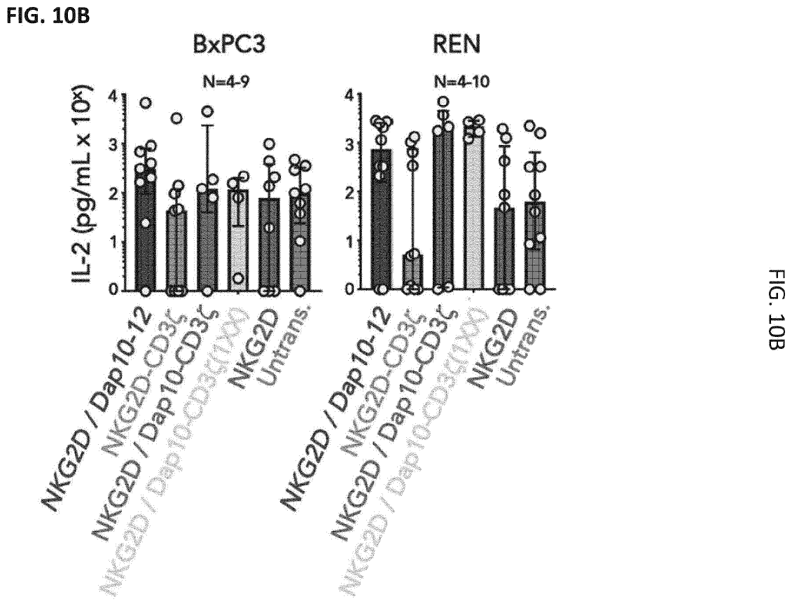

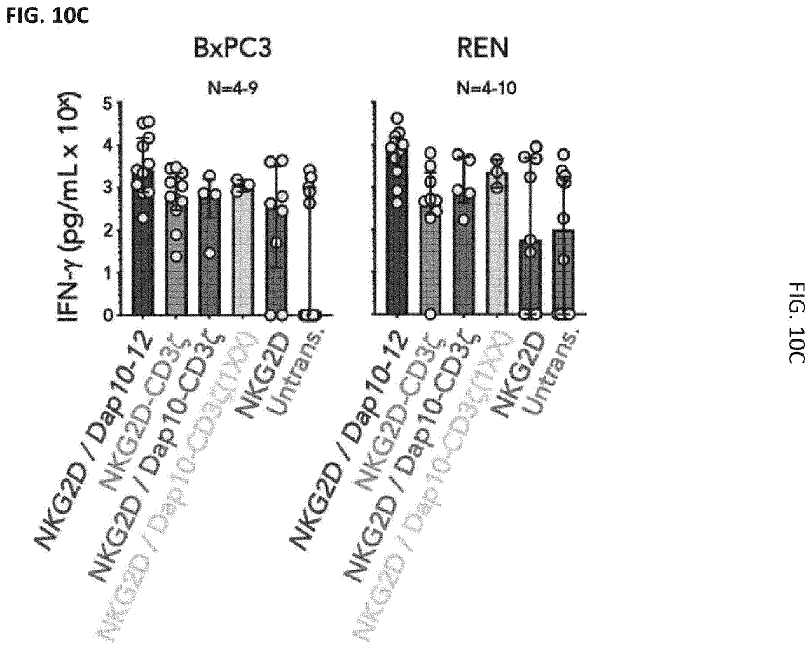

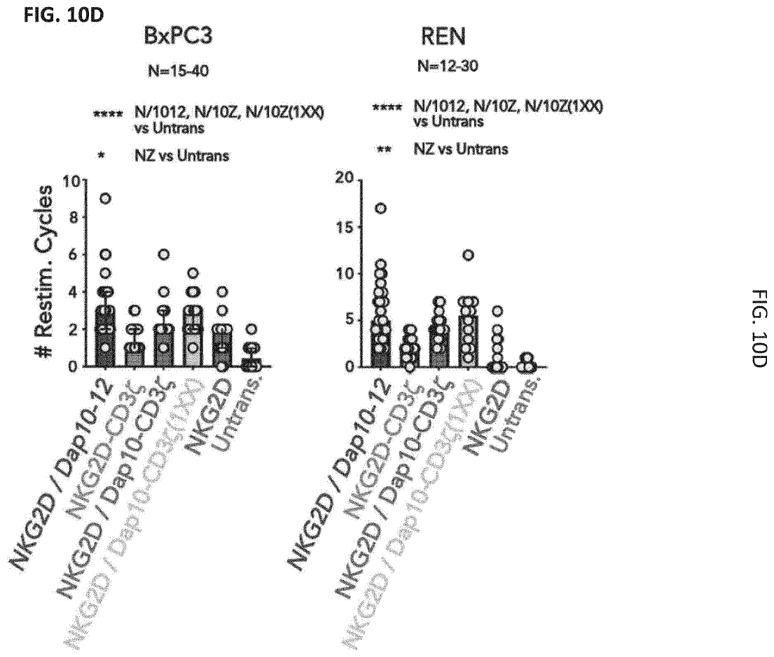

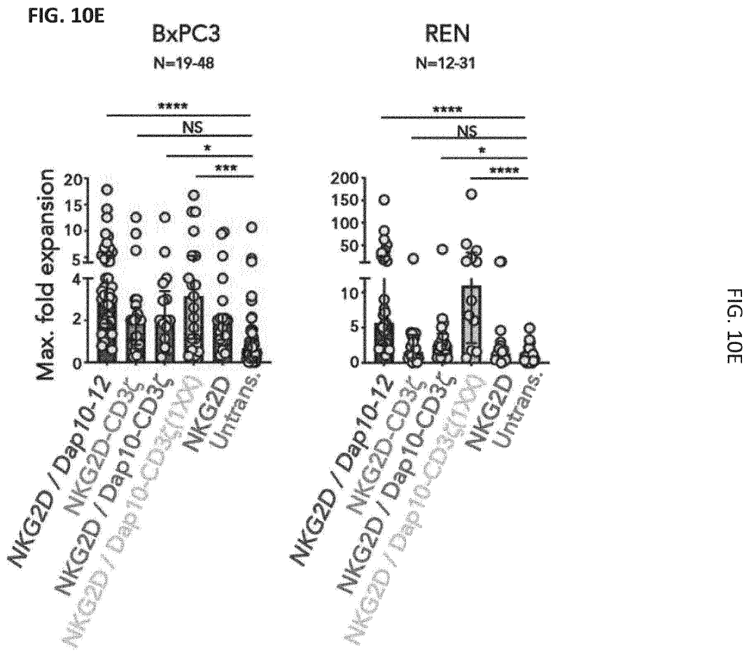

T-cells transduced with adaptor CARs expressing Dap10 demonstrated higher cell surface CAR expression compared to cells transduced with the linear CAR NKG2D-CD3ζ that relies on association with endogenous Dap10 (FIG. 9C). Results of in vitro assays evaluating cytotoxicity (FIG. 10A), cytokine release (FIGS. 10B-10C), and tumour re-stimulation (FIG. 10D) did not demonstrate any significant differences between the anti-tumour activity of the adaptor CARs (NKG2D/Dap10-12; NKG2D/Dap10-CD3ζ; NKG2D/Dap10-CD3ζ (1XX). Measurement of maximum fold T-cell expansion achieved during iterative restimulation on tumour cell monolayers demonstrated that NKG2D/Dap10-12 and NKG2D/Dap10-CD3ζ (1XX) both achieved the greatest expansion in these assays (FIG. 10E).

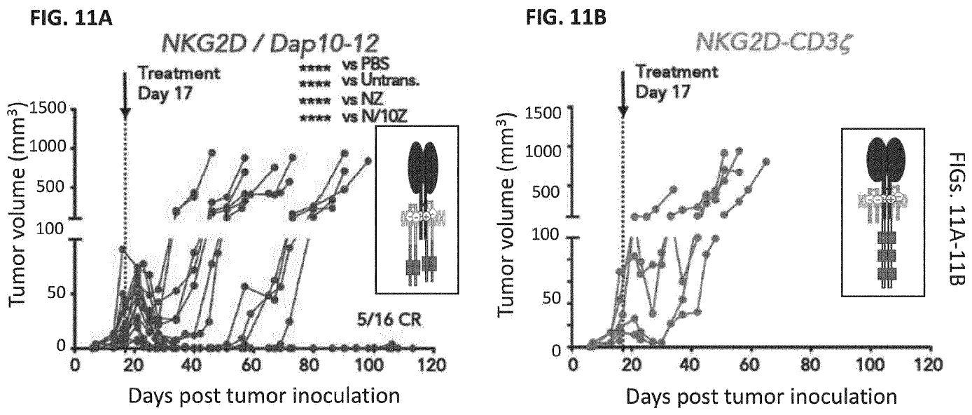

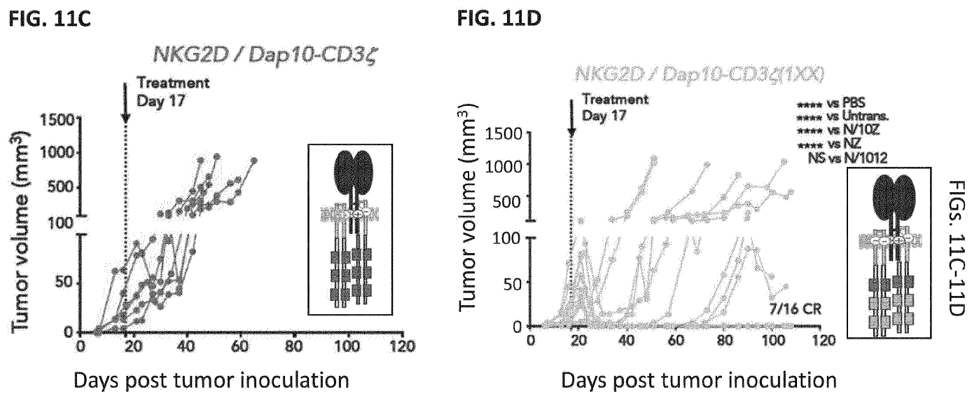

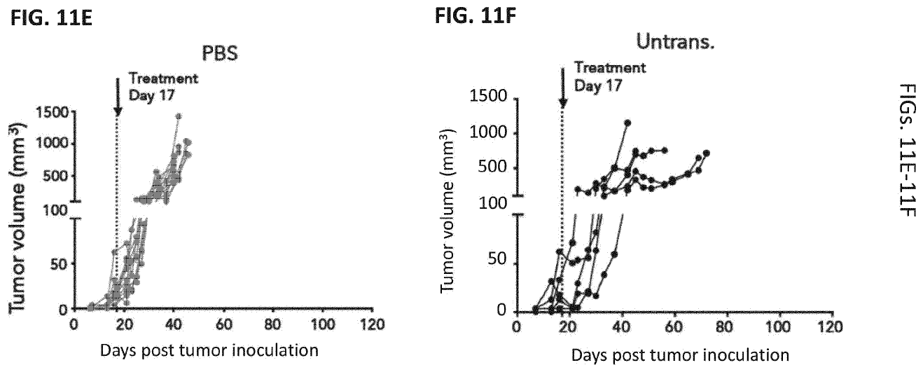

Next, we undertook in vivo comparison of these CARs in a subcutaneous BXPC3 xenograft model, making further comparison with untrans(duced) T-cell and PBS-treated mice. Tumours were established for 17 days prior to intravenous (i.v.) delivery of CAR T-cells. The results showed that anti-tumour activity fell into two groups. Significant anti-tumour activity was seen in mice treated with NKG2D/Dap10-12 (FIG. 11A) and NKG2D/Dap10-CD3ζ ((1XX) (FIG. 11D) CAR T-cells. In contrast, the NKG2D-CD3ζ (FIG. 11B) and NKG2D/Dap10-CD3ζ (FIG. 11C) CAR T-cells—which each have an activation signalling domain comprising three ITAMs—did not exert any significant anti-tumour activity when compared to untransduced T-cells (FIG. 11F). These data indicate that superior efficacy of adaptor CAR T-cells relies on reduced ITAM dosage. High cell surface CAR expression achieved by exogenous expression of Dap10 did not improve efficacy, as demonstrated by the poor anti-tumour activity seen in mice treated with NKG2D/Dap10-CD3ζ CAR T-cells (FIG. 11C).

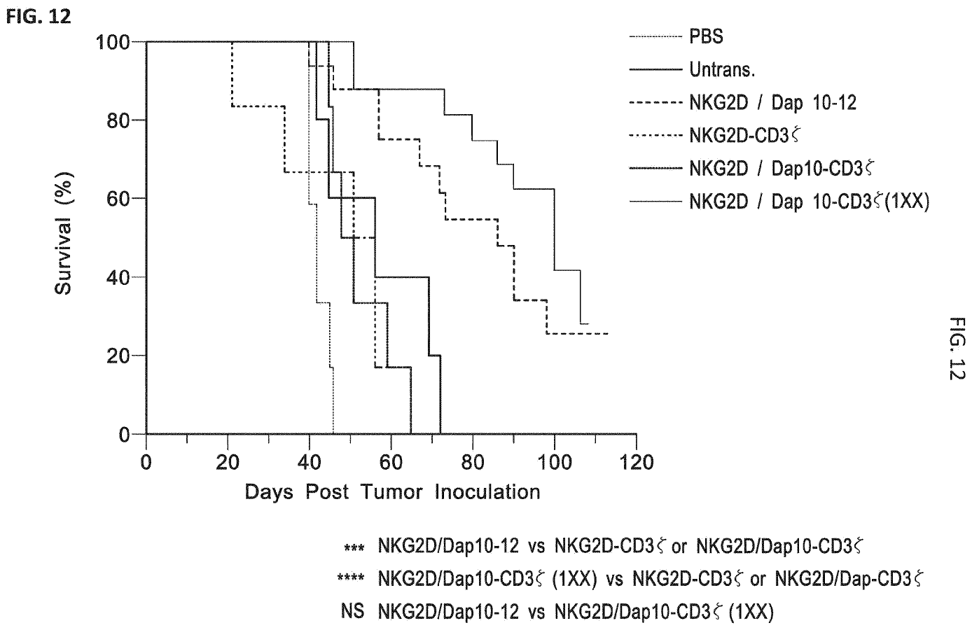

Survival of BXPC3 xenograft mice treated with NKG2D/Dap10-12; NKG2D/Dap10-CD3ζ; NKG2D/Dap10-CD3ζ (1XX); or NKG2D-CD3ζ CAR T-cells is shown in FIG. 12. The results show that survival of mice treated with NKG2D/Dap10-CD3ζ (1XX) or NKG2D/Dap10-12 T-cells was significantly prolonged.

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or document.

While the invention has been particularly shown and described with reference to a preferred embodiment and various alternate embodiments, it will be understood by persons skilled in the relevant art that various changes in form and details can be made therein without departing from the spirit and scope of the invention.

### 8. Embodiments

Embodiment 1. A second-generation chimeric antigen receptor (second-generation CAR) comprising:
a) an intracellular signalling domain comprising a modified CD3ζ polypeptide;
b) a co-stimulatory signalling region;
c) a transmembrane domain; and
d) a first binding element that specifically interacts with a first epitope on a first target antigen,
wherein the modified CD3ζ polypeptide comprises an unmodified immunoreceptor tyrosine-based activation motif 1 (ITAM1), and one or more mutations in ITAM2 and/or ITAM3.

Embodiment 2. The second-generation CAR of embodiment 1, wherein the modified CD3ζ polypeptide comprises mutations in both ITAM2 and ITAM3.

Embodiment 3. The second-generation CAR of embodiment 2, wherein the ITAM2 and the ITAM3 in the modified CD3ζ are non-functional.

Embodiment 4. The second-generation CAR of any one of embodiments 1-3, wherein each of the ITAM2 and the ITAM3 comprises at least one Tyr to Phe mutation.

Embodiment 5. The second-generation CAR of any one of embodiments 1-4, wherein each of the ITAM2 and the ITAM3 comprises two Tyr to Phe mutations.

Embodiment 6. The second-generation CAR of any one of embodiments 1-3, wherein the modified CD3ζ polypeptide comprises deletion of ITAM2 or a portion thereof and deletion of ITAM3 or a portion thereof.

Embodiment 7. The second-generation CAR of embodiment 6, wherein the modified CD3ζ polypeptide further comprises deletion of one or more amino acid residues on the N-terminal side of the ITAM1.

Embodiment 8. The second-generation CAR of any one of the preceding embodiments, wherein the modified CD3ζ polypeptide comprises a sequence selected from SEQ ID NOs: 48-53.

Embodiment 9. The second-generation CAR of any one of the preceding embodiments, wherein the first target antigen is selected from an NKG2D ligand (e.g., MICA, MICB, ULBP1-ULBP6), MUC1, αvβ6 integrin, ErbB, HER2, B7-H3, Claudin 18.2, Claudin 6, Glypican 3, anaplastic lymphoma kinase (ALK), CD70, and prostate-specific membrane antigen (PSMA).

Embodiment 10. The second-generation CAR of any one of the preceding embodiments, wherein the first target antigen is MUC1.

Embodiment 11. The second-generation CAR of any one of the preceding embodiments, wherein the first binding element comprises the CDRs of the HMFG2 antibody.

Embodiment 12. The second-generation CAR of embodiment 11, wherein the first binding element comprises the V_{H} and V_{L} domains of HMFG2 antibody.

Embodiment 13. The second-generation CAR of any one of the preceding embodiments, wherein the first binding element comprises HMFG2 single-chain variable fragment (scFv).

Embodiment 14. The second-generation CAR of any one of the preceding embodiments, wherein the second-generation CAR has at least 90% sequence identity to the amino acid sequence of SEQ ID NO: 59.

Embodiment 15. A parallel chimeric antigen receptor (pCAR) comprising the second-generation CAR according to any one of the preceding embodiments and a chimeric co-stimulatory receptor (CCR) comprising:
e) a co-stimulatory signalling region which is different from that of (b);
f) a transmembrane domain; and
g) a second binding element that specifically interacts with a second epitope on a second target antigen.

Embodiment 16. The second-generation CAR of any one of embodiments 1-14 or the pCAR of embodiment 15, wherein the co-stimulatory signalling region is selected from co-stimulatory signalling regions of members of the B7/CD28 family, the ILT/CD85 family, the tumour necrosis factor (TNF) superfamily, the SLAM family, and the TIM family.

Embodiment 17. The second-generation CAR of any one of embodiments 1-14 or the pCAR of embodiment 15 or embodiment 16, wherein the co-stimulatory signalling region is selected from a CD28 co-stimulatory domain, a CD27 co-stimulatory domain, an OX40 co-stimulatory domain, an ICOS co-stimulatory domain, and a 4-1BB co-stimulatory domain.

Embodiment 18. The second-generation CAR of any one of embodiments 1-14 or the pCAR of any one of embodiments 15-17, wherein the co-stimulatory signalling region of (b) is a CD28 co-stimulatory signalling domain.

Embodiment 19. The second-generation CAR or pCAR of embodiment 18, wherein the CD28 co-stimulatory domain comprises a mutation in the YRS motif.

Embodiment 20. The pCAR of any one of embodiments 15-19, wherein the co-stimulatory signalling region of (e) is a 4-1BB co-stimulatory domain.

Embodiment 21. The second-generation CAR of embodiment 1 or the pCAR of embodiment 15, wherein the co-stimulatory signalling region is a co-stimulatory molecule selected from CD7, CD96, CD160, CD200, CD300a, CRTAM, DAP10, DAP12, Dectin-1, DPPIV, EphB6, LAG-3, TSLPR, or a variant thereof.

Embodiment 22. The second-generation CAR of any one of embodiments 1-14 or the pCAR of any one of embodiments 15-21, wherein the transmembrane domain is selected from the transmembrane domains of CD8α, CD28, CD4, CD3ζ, FcεR1γ.

Embodiment 23. The second-generation CAR of any one of embodiments 1-14 or the pCAR of any one of claims 15-22, wherein the transmembrane domain of (c) is a CD28 transmembrane domain.

Embodiment 24. The pCAR of any one of embodiments 15-23, wherein the transmembrane domain of (f) is a CD8α transmembrane domain.

Embodiment 25. The pCAR of any one of embodiments 15-24, wherein the second target antigen is selected from an NKG2D ligand (e.g., MICA, MICB, ULBP1-ULBP6), MUC1, αvβ6 integrin, ErbB, HER2, B7-H3, GD2, Claudin 18.2, Claudin 18.6, Glypican 3, anaplastic lymphoma kinase (ALK), CD47, CD70, prostate-specific membrane antigen (PSMA), PD-L1, and NKp30 ligands (e.g., BAG6 and B7-H6).

Embodiment 26. The pCAR of any one of embodiments 15-25, wherein the second binding element specifically interacts with a second epitope on an ErbB antigen.

Embodiment 27. The pCAR of any one of embodiments 15-26, wherein the second binding element is a T1E peptide, an antigen binding site of ICR12, or an antigen binding site of ICR62.

Embodiment 28. The pCAR of any one of embodiments 15-27, wherein the second binding element is a T1E peptide.

Embodiment 29. The pCAR of any one of embodiments 15-25, wherein the second binding element specifically binds PD-L1.

Embodiment 30. The pCAR of any one of embodiments 15-25, wherein the second binding element specifically binds an NKp30 ligand selected from BAG6 and B7-H6.

Embodiment 31. The pCAR of any one of embodiments 15-30, wherein the second-generation CAR has at least 90% sequence identity to the amino acid sequence of SEQ ID NO: 59 and the CCR has at least 90% sequence identity to the amino acid sequence of SEQ ID NO: 24.

Embodiment 32. One or more polynucleotide constructs encoding the second-generation CAR of any one of embodiments 1-14 or the pCAR of any one of embodiments 15-31.

Embodiment 33. One or more expression vectors comprising the one or more polynucleotide constructs of embodiment 32.

Embodiment 34. An immunoresponsive cell comprising the second-generation CAR of any one of embodiments 1-14, the pCAR of any one of embodiments 15-31, the one or more polynucleotide constructs of embodiment 32, or the one or more expression vectors of embodiment 33.

Embodiment 35. The immunoresponsive cell of embodiment 34, wherein the immunoresponsive cell is an as T cell, 76 T cell, or a Natural Killer (NK) cell.

Embodiment 36. A pharmaceutical composition comprising the one or more polynucleotide constructs of embodiment 32, the one or more expression vectors of embodiment 33, or a population of immunoresponsive cells of embodiment 34 or embodiment 35 and an excipient.

Embodiment 37. A method for directing a T cell-mediated immune response to a target cell in a subject in need thereof, the method comprising administering to the subject a therapeutically effective amount of the one or more polynucleotide constructs of embodiment 32, the one or more expression vectors of embodiment 33, the immunoresponsive cell of embodiment 34 or 35 or the pharmaceutical composition of embodiment 36.

Embodiment 38. A method of treating cancer in a subject in need thereof, the method comprising administering a therapeutically effective amount of the one or more polynucleotide constructs of embodiment 32, the one or more expression vectors of embodiment 33, the immunoresponsive cell of embodiment 34 or 35 or the pharmaceutical composition of embodiment 36.

Embodiment 39. The method of embodiment 34 or 35, wherein the subject has cancer.

Embodiment 40. The method of embodiment 39, wherein the subject has a cancer selected from the group consisting of breast cancer, ovarian cancer, pancreatic cancer, colorectal cancer, lung cancer, gastric cancer, bladder cancer, myeloma, non-Hodgkin lymphoma, prostate cancer, oesophageal cancer, endometrial cancer, hepatobiliary cancer, duodenal carcinoma, thyroid carcinoma, and renal cell carcinoma.

Embodiment 41. The second-generation CAR of any one of embodiments 1-14, the pCAR of any one of embodiments 15-31, the one or more polynucleotide constructs of embodiment 32, the one or more expression vectors of embodiment 33, the immunoresponsive cell of embodiment 34 or 35, or the pharmaceutical composition of embodiment 37 for use in therapy.

Embodiment 42. The second-generation CAR of any one of embodiments 1-14, the pCAR of any one of embodiments 15-31, the one or more polynucleotide constructs of embodiment 32, the one or more expression vectors of embodiment 33, the immunoresponsive cell of embodiment 34 or 35, or the pharmaceutical composition of embodiment 37 for use in the treatment of cancer.

Embodiment 43. Use of the second-generation CAR of any one of embodiments 1-14, the pCAR of any one of embodiments 15-31, the one or more polynucleotide constructs of embodiment 32, the one or more expression vectors of embodiment 33, the immunoresponsive cell of embodiment 34 or 35, or the pharmaceutical composition of embodiment 37 for the manufacture of a medicament for the treatment of cancer.

What is claimed is:

1. A parallel chimeric antigen receptor (pCAR) comprising:
(1) a second-generation chimeric antigen receptor (second-generation CAR) comprising:
a) an intracellular signalling domain comprising a modified CD3ζ polypeptide;
b) a co-stimulatory signalling region;
c) a transmembrane domain; and
d) a first binding element that specifically interacts with a first epitope on a first target antigen,
wherein the modified CD3ζ polypeptide comprises an unmodified immunoreceptor tyrosine-based activation motif 1 (ITAM1), and one or more mutations in ITAM2 and/or ITAM3; and
(2) a chimeric co-stimulatory receptor (CCR) comprising at least one CCR polypeptide comprising:
e) a co-stimulatory signalling region which is different from that of (b);
f) a transmembrane domain; and
g) a second binding element that specifically interacts with a second epitope on a second target antigen.

2. The pCAR of claim 1, wherein the modified CD3ζ polypeptide comprises mutations in both ITAM2 and ITAM3.

3. The pCAR of claim 2, wherein the ITAM2 and the ITAM3 in the modified CD3ζ are non-functional.

4. The pCAR of any one of claims 1-3, wherein (i) each of the ITAM2 and the ITAM3 comprises at least one Tyr to Phe mutation or two Tyr to Phe mutations or (ii) the modified CD3ζ polypeptide comprises deletion of ITAM2 or a portion thereof and deletion of ITAM3 or a portion thereof.

5. The pCAR of claim 4, wherein the modified CD3ζ polypeptide further comprises deletion of one or more amino acid residues on the N-terminal side of the ITAM1.

6. The pCAR of any one of the preceding claims, wherein the modified CD3ζ polypeptide comprises a sequence selected from SEQ ID NOs: 48-53.

7. The pCAR of any one of the preceding claims, wherein the first target antigen is selected from an NKG2D ligand (e.g., MICA, MICB, ULBP1-ULBP6), MUC1, αvβ6 integrin, ErbB, HER2, B7-H3, Claudin 18.2, Claudin 18.6, Glypican 3, ALK, CD70, and prostate-specific membrane antigen (PSMA).

8. The pCAR of any one of the preceding claims, wherein the first binding element comprises the CDRs of the HMFG2 antibody, the V_{H} and V_{L} domains of HMFG2 antibody or HMFG2 single-chain variable fragment (scFv).

9. The pCAR of any one of the preceding claims, wherein the second-generation CAR has at least 90% sequence identity to the amino acid sequence of SEQ ID NO: 59.

10. The pCAR of any one of the preceding claims, wherein the co-stimulatory signalling region is selected from (i) co-stimulatory signalling regions of members of the B7/CD28 family, the ILT/CD85 family, the tumour necrosis factor (TNF) superfamily, the SLAM family, and the TIM family, (ii) a CD28 co-stimulatory domain, a 4-1BB co-stimulatory domain, an OX40 co-stimulatory domain, and an ICOS co-stimulatory domain, or (iii) a co-stimulatory molecule selected from CD7, CD96, CD160, CD200, CD300a, CRTAM, DAP10, DAP12, Dectin-1, DPPIV, EphB6, LAG-3, TSLP R, or a variant thereof.

11. The pCAR of claim 10, wherein the CD28 co-stimulatory domain comprises a mutation in the YRS motif.

12. The pCAR of any one of the preceding claims, wherein the co-stimulatory signalling region of (e) is a 4-1BB co-stimulatory domain.

13. The pCAR of any one of the preceding claims, wherein the transmembrane domain is selected from the transmembrane domains of CD8α, CD28, CD4, CD3ζ, FcεR1γ.

14. The pCAR of any one of the preceding claims, wherein the transmembrane domain of (c) is a CD28 transmembrane domain and/or the transmembrane domain of (f) is a CD8α transmembrane domain.

15. The pCAR of any one of the preceding claims, wherein the second target antigen is selected from an NKG2D ligand (e.g., MICA, MICB, ULBP1-ULBP6), MUC1, αvβ6 integrin, ErbB, HER2, B7-H3, GD2, Claudin 18.2, Claudin 18.6, Glypican 3, ALK, CD47, CD70, prostate-specific membrane antigen (PSMA), a PD-1 ligand (e.g., PD-L1), NKp44 ligands (e.g., NKp44L (MLL5), PCNA, viral haemagglutinins, nidogen-1, galectin-3, other proteoglycans) and NKp30 ligands (e.g., BAG6, B7-H6).

16. The pCAR of any one of the preceding claims, wherein the second binding element specifically interacts with an ErbB antigen, an NKG2D ligand, a PD-1 ligand, an NKp44 ligand, or an NKp30 ligand.

17. The pCAR of any one of the preceding claims, wherein the second binding element is any one of a T1E peptide, an NKG2D polypeptide, a PD-1 polypeptide, an NKp44 polypeptide, an NKp30 polypeptide, or a fragment, portion, or variant thereof.

18. The pCAR of any one of the preceding claims, wherein the CCR of (2) comprises two or more CCR polypeptides.

19. The pCAR of any one of the preceding claims, wherein the CCR of (2) comprises three CCR polypeptides.

20. The pCAR of claim 18, wherein the CCR comprises two CCR polypeptides that dimerize following interaction of the second binding element with the second target antigen.

21. The pCAR of claim 19, wherein the three CCR polypeptides trimerize following interaction of the second binding element with the second target antigen.

22. The pCAR of any one of the preceding claims, wherein the second-generation CAR has at least 90% sequence identity to the amino acid sequence of SEQ ID NO: 59 and the CCR has at least 90% sequence identity to the amino acid sequence of any one of SEQ ID NO: 24, SEQ ID NO: 77, SEQ ID NO: 79, SEQ ID NO: 81, and SEQ ID NO: 83.

23. One or more polynucleotide constructs encoding the pCAR of any one of the preceding claims.

24. One or more expression vectors comprising the one or more polynucleotide constructs of claim 23.

25. An immunoresponsive cell comprising the pCAR of any one of claims 1-22, the one or more polynucleotide constructs of claim 23, or the one or more expression vectors of claim 24.

26. The immunoresponsive cell of claim 25, wherein the immunoresponsive cell is an as T cell, γδ T cell, or a Natural Killer (NK) cell.

27. A pharmaceutical composition comprising the one or more polynucleotide constructs of claim 23, the one or more expression vectors of claim 24, or a population of immunoresponsive cells of claim 25 or claim 26 and an excipient.

28. The pCAR of any one of claims 1-22, the one or more polynucleotide constructs of claim 23, the one or more expression vectors of claim 24, the immunoresponsive cell of claim 25 or 26, or the pharmaceutical composition of claim 27 for use in therapy.

29. The pCAR of any one of claims 1-22, the one or more polynucleotide constructs of claim 23, the one or more expression vectors of claim 24, the immunoresponsive cell of claim 25 or 26, or the pharmaceutical composition of claim 27 for use in the treatment of cancer.

30. A fusion polypeptide comprising (i) a DNAX-activating protein 10 (Dap10) polypeptide, or a functional variant thereof and (ii) a modified CD3ζ polypeptide that comprises an unmodified immunoreceptor tyrosine-based activation motif 1 (ITAM1), and one or more mutations in ITAM2 and/or ITAM3.

31. The fusion polypeptide of claim 30, wherein the Dap10 polypeptide has at least 90% sequence identity to the amino acid sequence of SEQ ID NO: 47.

32. The fusion polypeptide of claim 30 or claim 31, wherein the modified CD3ζ polypeptide comprises mutations in both ITAM2 and ITAM3.

33. The fusion polypeptide of any one of claims 30-32, wherein the ITAM2 and the ITAM3 in the modified CD3ζ are non-functional.

34. The fusion polypeptide of any one of claims 30-33, wherein (i) each of the ITAM2 and the ITAM 3 comprises at least one Tyr to Phe mutation or two Tyr to Phe mutations or (ii) the modified CD3ζ polypeptide comprises deletion of ITAM2 or a portion thereof and deletion of ITAM3 or a portion thereof.

35. The fusion polypeptide of any one of claims 30-34, wherein the modified CD3ζ polypeptide has at least 95% sequence identity to the amino acid sequence of SEQ ID NO: 48.
